SEQUENCE LISTING (29 sequences)

SEQ ID NO: 1 - DNA, 1000 bp, synthetic construct
  source 1..1000: mol_type = other DNA
cagtgagcttcattatcacctaacagcttcagagtgggtggtgggttttggatgacaacctttcttctcattttattcagtggccacaccgtggccttagtctgataaaccaaaaacctgctccattatgaatcagtgctgtggggagtgggtagagagtgtgaagttctggggtgggggagtctggagagagggtgggagcagccattctgcagcagtgccttcttggggtcatgggtctgtaggtgctgctgtggagggagagatcagcctattctggcttcatttctgagctgcaaactgcctgggtgtctggagaagcaggttggcgtggtggttagcagtgcgtgggcggggttgcccgctcttgatttatgatttctttgtctctgtggaagcacttaagtgcaggctttagttccaatgacactcaggagcctctggattccagcactggggatgggggtggggtagaacgttctcaggcctcaccaacccctcccctgtgtgctgcctttgggagagtcccaaggcttcagcattacttaattaattaggcctctactgctacataggctcagattcaaaagaacagagtggcccacgtcagccattcccggaaaagtctgatggctggaagccagaggactatgtgtctgccttgctgcccttggccagcccatcctgaatgcccagactcggacaatggagtaggtacagaagggtaaagacagtgtcttctgtaccagtaagtgggccctgatctgctctctacagcttccagagaaagggcctggccaatgagcggccttttgagtagcagatacctcacatgcattctgatagaaagcctggccccagatcactgtgactttagccctcaggtttcttttgcacttcaattcaatgacttcttgaggttcatttccctctccaagatttgccacagaccagtggttctcaacctgtgggtcacacctcctttggggaaattgaatga

SEQ ID NO: 2 - DNA, 861 bp, synthetic construct
  source 1..861: mol_type = other DNA
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 3 - protein, 286 aa, synthetic construct
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 4 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 5 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 6 - DNA, 32 bp, synthetic construct
  source 1..32: mol_type = other DNA
tagagggtatataatggaagctcgacttccag

SEQ ID NO: 7 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 8 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 9 - DNA, 122 bp, synthetic construct
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 10 - DNA, 6010 bp, synthetic construct
  source 1..6010: mol_type = other DNA
cttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagtaggtacccagtgagcttcattatcacctaacagcttcagagtgggtggtgggttttggatgacaacctttcttctcattttattcagtggccacaccgtggccttagtctgataaaccaaaaacctgctccattatgaatcagtgctgtggggagtgggtagagagtgtgaagttctggggtgggggagtctggagagagggtgggagcagccattctgcagcagtgccttcttggggtcatgggtctgtaggtgctgctgtggagggagagatcagcctattctggcttcatttctgagctgcaaactgcctgggtgtctggagaagcaggttggcgtggtggttagcagtgcgtgggcggggttgcccgctcttgatttatgatttctttgtctctgtggaagcacttaagtgcaggctttagttccaatgacactcaggagcctctggattccagcactggggatgggggtggggtagaacgttctcaggcctcaccaacccctcccctgtgtgctgcctttgggagagtcccaaggcttcagcattacttaattaattaggcctctactgctacataggctcagattcaaaagaacagagtggcccacgtcagccattcccggaaaagtctgatggctggaagccagaggactatgtgtctgccttgctgcccttggccagcccatcctgaatgcccagactcggacaatggagtaggtacagaagggtaaagacagtgtcttctgtaccagtaagtgggccctgatctgctctctacagcttccagagaaagggcctggccaatgagcggccttttgagtagcagatacctcacatgcattctgatagaaagcctggccccagatcactgtgactttagccctcaggtttcttttgcacttcaattcaatgacttcttgaggttcatttccctctccaagatttgccacagaccagtggttctcaacctgtgggtcacacctcctttggggaaattgaatgaaccggtctagagggtatataatggaagctcgacttccaggtaagtatcaaggttacaagacaggtttaaggagaccaatagaaactgggcttgtcgagacagagaagactcttgcgtttctgataggcacctattggtcttactgacatccactttgcctttctctccacagggcaaagaattggatccatgggagacatgggagatccaccaaaaaaaaaacgtctgatttccctatgtgttggttgcggcaatcagattcacgatcggtatattctgagggtttctccggatttggaatggcatgcggcatgtttgaaatgtgcggagtgtaatcagtatttggacgagagctgtacatgctttgttagggatgggaaaacctactgtaaaagagattatatcaggttgtacgggatcaaatgcgccaagtgcagcatcggcttcagcaagaacgacttcgtgatgcgtgcccgctccaaggtgtatcacatcgagtgtttccgctgtgtggcctgcagccgccagctcatccctggggacgaatttgcgcttcgggaggacggtctcttctgccgagcagaccacgatgtggtggagagggccagtctaggcgctggcgacccgctcagtcccctgcatccagcgcggccactgcaaatggcagcggagcccatctccgccaggcagccggccctgcggccccacgtccacaagcagccggagaagaccacccgcgtgcggactgtgctgaacgagaagcagctgcacaccttgcggacctgctacgccgcaaacccgcggccagatgcgctcatgaaggagcaactggtagagatgacgggcctcagtccccgtgtgatccgggtctggtttcaaaacaagcggtgcaaggacaagaagcgaagcatcatgatgaagcaactccagcagcagcagcccaatgacaaaactaatatccaggggatgacaggaactcccatggtggctgccagtccagagagacacgacggtggcttacaggctaacccagtggaagtacaaagttaccagccaccttggaaagtactgagcgacttcgccttgcagagtgacatagatcagcctgcttttcagcaactggtcaatttttcagaaggaggaccgggctctaattccactggcagtgaagtagcatcaatgtcctctcaacttccagatacacctaacagcatggtagccagtcctattgaggcataggatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgct

SEQ ID NO: 11 - DNA, 6175 bp, synthetic construct
  source 1..6175: mol_type = other DNA
cttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagtaggtacccagtgagcttcattatcacctaacagcttcagagtgggtggtgggttttggatgacaacctttcttctcattttattcagtggccacaccgtggccttagtctgataaaccaaaaacctgctccattatgaatcagtgctgtggggagtgggtagagagtgtgaagttctggggtgggggagtctggagagagggtgggagcagccattctgcagcagtgccttcttggggtcatgggtctgtaggtgctgctgtggagggagagatcagcctattctggcttcatttctgagctgcaaactgcctgggtgtctggagaagcaggttggcgtggtggttagcagtgcgtgggcggggttgcccgctcttgatttatgatttctttgtctctgtggaagcacttaagtgcaggctttagttccaatgacactcaggagcctctggattccagcactggggatgggggtggggtagaacgttctcaggcctcaccaacccctcccctgtgtgctgcctttgggagagtcccaaggcttcagcattacttaattaattaggcctctactgctacataggctcagattcaaaagaacagagtggcccacgtcagccattcccggaaaagtctgatggctggaagccagaggactatgtgtctgccttgctgcccttggccagcccatcctgaatgcccagactcggacaatggagtaggtacagaagggtaaagacagtgtcttctgtaccagtaagtgggccctgatctgctctctacagcttccagagaaagggcctggccaatgagcggccttttgagtagcagatacctcacatgcattctgatagaaagcctggccccagatcactgtgactttagccctcaggtttcttttgcacttcaattcaatgacttcttgaggttcatttccctctccaagatttgccacagaccagtggttctcaacctgtgggtcacacctcctttggggaaattgaatgaaccggtctagagggtatataatggaagctcgacttccaggtaagtatcaaggttacaagacaggtttaaggagaccaatagaaactgggcttgtcgagacagagaagactcttgcgtttctgataggcacctattggtcttactgacatccactttgcctttctctccacagggcaaagaattggatccatggaggcgcgcggggagctgggcccggcccgggagtcggcgggaggcgacctgctgctagcactgctggcgcggagggcggacctgcgccgagagatcccgctgtgcgctggctgtgaccagcacatcctggaccgcttcatcctcaaggctctggaccgccactggcacagcaagtgtctcaagtgcagcgactgccacacgccactggccgagcgctgcttcagccgaggggagagcgtttactgcaaggacgactttttcaagcgcttcgggaccaagtgcgccgcgtgccagctgggcatcccgcccacgcaggtggtgcgccgcgcccaggacttcgtgtaccacctgcactgctttgcctgcgtcgtgtgcaagcggcagctggccacgggcgacgagttctacctcatggaggacagccggctcgtgtgcaaggcggactacgaaaccgccaagcagcgagaggccgaggccacggccaagcggccgcgcacgaccatcaccgccaagcagctggagacgctgaagagcgcttacaacacctcgcccaagccggcgcgccacgtgcgcgagcagctctcgtccgagacgggcctggacatgcgcgtggtgcaggtttggttccagaaccgccgggccaaggagaagaggctgaagaaggacgccggccggcagcgctgggggcagtatttccgcaacatgaagcgctcccgcggcggctccaagtcggacaaggacagcgttcaggaggggcaggacagcgacgctgaggtctccttccccgatgagccttccttggcggaaatgggcccggccaatggcctctacgggagcttgggggaacccacccaggccttgggccggccctcgggagccctgggcaacttctccctggagcatggaggcctggcaggcccagagcagtaccgagagctgcgtcccggcagcccctacggtgtccccccatcccccgccgccccgcagagcctccctggcccccagcccctcctctccagcctggtgtacccagacaccagcttgggccttgtgccctcgggagcccccggcgggcccccacccatgagggtgctggcagggaacggacccagttctgacctatccacggggagcagcgggggttaccccgacttccctgccagccccgcctcctggctggatgaggtagaccacgctcagttctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgct

SEQ ID NO: 12 - protein, 284 aa, synthetic construct
MDYSYLNSYDSCVAAMEASAYGDFGACSQPGGFQYSPLRPAFPAAGPPCPALGSSNCALGALRDHQPAPYSAVPYKFFPEPSGLHEKRKQRRIRTTFTSAQLKELERVFAETHYPDIYTREELALKIDLTEARVQVWFQNRRAKFRKQERAASAKGAAGAAGAKKGEARCSSEDDDSKESTCSPTPDSTASLPPPPAPGLASPRLSPSPLPVALGSGPGPGPGPQPLKGALWAGVAGGGGGGPGAGAAELLKAWQPAESGPGPFSGVLSSFHRKPGPALKTNLF

SEQ ID NO: 13 - protein, 360 aa, synthetic construct
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 14 - protein, 317 aa, synthetic construct
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 15 - protein, 4 aa, synthetic construct
IEGR

SEQ ID NO: 16 - DNA, 50 bp, synthetic construct
  source 1..50: mol_type = other DNA
cctttctctccacagggcaaagaattggatccatggaggcgcgcggggag

SEQ ID NO: 17 - DNA, 50 bp, synthetic construct
  source 1..50: mol_type = other DNA
agaggttgattatcgataagcttgatatcttagaactgagcgtggtctac

SEQ ID NO: 18 - DNA, 50 bp, synthetic construct
  source 1..50: mol_type = other DNA
cctttctctccacagggcaaagaattggatccatgggagacatgggagat

SEQ ID NO: 19 - DNA, 50 bp, synthetic construct
  source 1..50: mol_type = other DNA
agaggttgattatcgataagcttgatatcctatgcctcaataggactggc

SEQ ID NO: 20 - DNA, 57 bp, synthetic construct
  source 1..57: mol_type = other DNA
gaattggatccgccactcggagcaagcttgatttaggtgacactatagaatacaagc

SEQ ID NO: 21 - DNA, 42 bp, synthetic construct
  source 1..42: mol_type = other DNA
catgtctgctcgaagcgtcagaactgagcatggtctacttca

SEQ ID NO: 22 - DNA, 20 bp, synthetic construct
  source 1..20: mol_type = other DNA
ggctgttgggcactgacaat

SEQ ID NO: 23 - DNA, 21 bp, synthetic construct
  source 1..21: mol_type = other DNA
ccgaagggacgtagcagaagg

SEQ ID NO: 24 - DNA, 23 bp, synthetic construct
  source 1..23: mol_type = other DNA
tccactggcagtgaagtagcatc

SEQ ID NO: 25 - DNA, 24 bp, synthetic construct
  source 1..24: mol_type = other DNA
aaggatgaaacggtccaagatgtg

SEQ ID NO: 26 - DNA, 21 bp, synthetic construct
  source 1..21: mol_type = other DNA
catcagccctaatccatctga

SEQ ID NO: 27 - DNA, 21 bp, synthetic construct
  source 1..21: mol_type = other DNA
cgcgactaacaatcaaagtga

SEQ ID NO: 28 - DNA, 19 bp, synthetic construct
  source 1..19: mol_type = other DNA
caacagggtggtggacctc

SEQ ID NO: 29 - DNA, 20 bp, synthetic construct
  source 1..20: mol_type = other DNA
gggtggtccagggtttctta

(19) United States
(12) Patent Application Publication
(10) Pub. No.: US 2025/0339563 A1
(43) Pub. Date: Nov. 6, 2025

(54) RE-EXPRESSION OF EMBRYONIC MOTOR NEURON TRANSCRIPTION FACTORS IN POST-NATAL ANIMALS AS A THERAPEUTIC STRATEGY FOR AMYOTROPHIC LATERAL SCLEROSIS

(71) Applicant: The Trustees of Columbia University in the City of New York, New York, NY (US)

(72) Inventors: Hynek WICHTERLE, New York, NY (US); Tulsi PATEL, New York, NY (US); Emily Rhodes LOWRY, New York, NY (US)

(21) Appl. No.: 19/272,917

(22) Filed: Jul. 17, 2025

Related U.S. Application Data
(63) Continuation-in-part of application No. PCT/US24/10757, filed on Jan. 8, 2024.
Application No. PCT/US24/10757 is a continuation of application No. PCT/US23/69780, filed on Jul. 7, 2023.
(60) Provisional application No. 63/480,490, filed on Jan. 18, 2023.
(60) Provisional application No. 63/481,308, filed on Jan. 24, 2023.

(51) Int. Cl. A61K 48/00; C12N 15/86

(52) U.S. Cl. CPC A61K 48/0058; C12N 15/86; C12N 2750/14143

(57) ABSTRACT

The subject matter described here relates to methods, compositions, and vectors for treating Amyotrophic Lateral Sclerosis (ALS) in a subject in need thereof. In certain aspects, the method comprises administering to the subject a composition comprising adeno-associated viruses (AAVs), wherein the AAVs comprise a nucleic acid sequence comprising an enhancer sequence and encoding one or more transcription factors that control gene expression in nascent motor neurons, wherein the enhancer is capable of driving a motor-neuron specific expression of the one or more transcription factors and wherein the one or more transcription factors are expressed in motor neurons of the subject.

### CROSS-REFERENCE TO RELATED APPLICATIONS

This application is a continuation in part of International Patent Application PCT/US2024/10757, filed on Jan. 8, 2024, which claims the benefit of and priority to U.S. Application No. 63/480,490, filed Jan. 18, 2023, and U.S. Application No. 63/481,308, filed Jan. 24, 2023, each entitled “RE-EXPRESSION OF EMBRYONIC MOTOR NEURON TRANSCRIPTION FACTORS IN POST-NATAL ANIMALS AS A THERAPEUTIC STRATEGY FOR AMYOTROPHIC LATERAL SCLEROSIS”, and the International Application No. PCT/US23/69780, filed Jul. 7, 2023, entitled “REGULATORY ELEMENT FOR CELL TYPE SPECIFIC EXPRESSION OF GENES IN SPINAL MOTOR NEURONS”, the contents of each of which are hereby incorporated by reference in their entireties.

### GOVERNMENT SUPPORT

This invention was made with government support under NS105372 and NS116141 awarded by the National Institutes of Health. The government has certain rights in the invention.

All patents, patent applications and publications cited herein are hereby incorporated by reference in their entirety. The disclosures of this provisional application in its entirety are hereby incorporated by reference into this application. In the event of a conflict between the teachings of the application and those of the incorporated provisional application, the teachings of the application will control.

This patent disclosure contains material that is subject to copyright protection. The copyright owner has no objection to the facsimile reproduction by anyone of the patent document or the patent disclosure as it appears in the U.S. Patent and Trademark Office patent file or records but otherwise reserves any and all copyright rights.

### SEQUENCE LISTING

The instant application contains a Sequence Listing which has been submitted electronically in XML format and is hereby incorporated by reference in its entirety. Said XML copy, created on Jul. 16, 2025, is named 0019240-01304US3_SL.xml and is 42,796 bytes in size.

### BACKGROUND

Amyotrophic lateral sclerosis (ALS) is a neurodegenerative disorder that is characterized by widespread motor neuron dysfunction and death. Aging is a key risk factor in developing the disease, where the average age of onset is ˜65 years. Though familial ALS patients harbor ALS-causing mutations throughout their whole lives, they typically do not fall ill until middle age. One explanation for this phenomenon could be that motor neurons in early stages of life are resilient and can initially resist the damage brought on by ALS-causing mutations, but their defenses wear down with age.

The transcription factors Islet1 and Lhx3 (Isl1/Lhx3) play a major role in spinal motor neuron specification during embryonic development, when motor neurons are resistant to ALS-causing mutations, and are downregulated in spinal motor neurons during postnatal life, when motor neurons become susceptible to disease. Therefore there is a need for “turning back the clock” in aged motor neurons to reactivate their native defenses and prevent motor neuron degeneration in ALS.

Disclosed herein is a method for driving the expression of Isl1 and/or Lhx3 in spinal motor neurons during postnatal life to reduce the cellular phenotypes associated with ALS in SOD1^{G93A} mice.

### SUMMARY

In certain aspects, the present disclosure provides a method for treating Amyotrophic Lateral Sclerosis (ALS) in a subject in need thereof, the method comprising: administering to the subject a composition comprising adeno-associated viruses (AAVs), wherein the AAVs comprise a nucleic acid sequence comprising an enhancer sequence and encoding one or more transcription factors that control gene expression in nascent motor neurons, wherein the enhancer is capable of driving a motor-neuron specific expression of the one or more transcription factors and wherein the one or more transcription factors are expressed in motor neurons of the subject.

In some embodiments, the motor neurons are rejuvenated, and/or their resistance to ALS pathogens in the subject with ALS is increased.

In some embodiments, the enhancer sequence comprises SEQ ID NO: 1. In some embodiments, the enhancer sequence consists of SEQ ID NO: 1. In some embodiments, the enhancer is ChatE.

In some embodiments, the one or more transcription factors is Lhx3. In some embodiments, the one or more transcription factors is Isl1. In some embodiments, the one or more transcription factors are Isl1 and Lhx3. In some embodiments, the nucleic acid sequence encoding Isl1 comprises nucleotides 3844-4890 of SEQ ID NO: 10. In some embodiments, the nucleic acid sequence encoding Isl1 comprises a nucleotide sequence encoding an amino acid sequence as encoded by nucleotides 3844-4890 of SEQ ID NO: 10. In some embodiments, the nucleic acid sequence encoding Isl1 consists of nucleotides 3844-4890 of SEQ ID NO: 10. In some embodiments, the nucleic acid sequence encoding Lhx3 comprises nucleotides 3844-5052 of SEQ ID NO: 11. In some embodiments, the nucleic acid sequence encoding Lhx3 comprises a nucleotide sequence encoding an amino acid sequence as encoded by nucleotides 3844-5052 of SEQ ID NO: 11. In some embodiments, the nucleic acid sequence encoding Lhx3 consists of nucleotides 3844-5052 of SEQ ID NO: 11. In some embodiments, the one or more transcription factors is Phox2a. In some embodiments, the one or more transcription factors is Sox2. In some embodiments, the one or more transcription factors are Phox2a and Isl1. In some embodiments, the one or more transcription factors is Oct4. In some embodiments, the one or more transcription factors are Oct4 and Sox2. In some embodiments, the nucleic acid sequence encoding Phox2a comprises a nucleotide sequence encoding an amino acid sequence of SEQ ID NO: 12. In some embodiments, the nucleic acid sequence encoding Oct4 comprises a nucleotide sequence encoding an amino acid sequence of SEQ ID NO: 13. In some embodiments, the nucleic acid sequence encoding Sox2 comprises a nucleotide sequence encoding an amino acid sequence of SEQ ID NO: 14.

In some embodiments, the AAVs comprise a nucleic acid comprising the nucleic acids between the AAV ITRs of SEQ ID NO:10 (i.e., nucleotides 2459-5955). In some embodiments, the AAVs comprise a nucleic acid consisting of the nucleic acids between the AAV ITRs of SEQ ID NO:10 (i.e., nucleotides 2459-5955). In some embodiments, the AAVs comprise a nucleic acid comprising the nucleic acids between the AAV ITRs of SEQ ID NO:11 (i.e., nucleotides 2459-6120). In some embodiments, the AAVs comprise a nucleic acid consisting of the nucleic acids between the AAV ITRs of SEQ ID NO:11 (i.e., nucleotides 2459-6120). In some embodiments, the AAVs comprise a nucleic acid comprising the nucleic acids between the AAV ITRs of SEQ ID NO:10 (i.e., nucleotides 2459-5955), except that the CDS of Isl1 is replaced with a nucleic acid sequence encoding Phox2a. In some embodiments, the AAVs comprise a nucleic acid comprising the nucleic acids between the AAV ITRs of SEQ ID NO:10 (i.e., nucleotides 2459-5955), except that the CDS of Isl1 is replaced with a nucleic acid sequence encoding Oct4. In some embodiments, the AAVs comprise a nucleic acid comprising the nucleic acids between the AAV ITRs of SEQ ID NO:10 (i.e., nucleotides 2459-5955), except that the CDS of Isl1 is replaced with a nucleic acid sequence encoding Sox2.

In some embodiments, the re-expression of one or more transcription factors reactivates their embryonic targets. In some embodiments, the embryonic target MNX1.

In some embodiments, the motor neurons are spinal motor neurons.

In some embodiments, the AAVs are capable of penetrating the blood-brain barrier.

In some embodiments, administration of the AAVs attenuates disease-related proteinopathies in the motor neurons. In some embodiments, administration of the AAVs reduces the formation of p62+ aggregates in the motor neurons. In some embodiments, administration of the AAVs reduces the incidence of SQSTM1-positive round bodies. In some embodiments, administration of the AAVs reduces the formation of SOD1+ aggregates in the motor neurons. In some embodiments, administration of the AAVs results in the reduction of neuroinflammation in the vicinity of motor neurons. In some embodiments, administration of the AAVs results in the reduction of Iba1+ microglia activation in the vicinity of the motor neurons.

In some embodiments, administration of the AAVs ameliorates clinical phenotypes of ALS. In some embodiments, administration of the AAVs delays symptom onset of ALS.

In certain aspects, the present disclosure provides a method for treating Amyotrophic Lateral Sclerosis (ALS) in a subject in need thereof, the method comprising: administering to the subject a first composition comprising adeno-associated viruses (AAVs) and a second composition comprising AAVs, wherein the AAVs of the first composition comprise a nucleic acid sequence comprising an enhancer sequence and encoding a first transcription factor that controls gene expression in nascent motor neurons and wherein the enhancer is capable of driving a motor-neuron specific expression of the first transcription factor, wherein the AAVs of the second composition comprise a nucleic acid sequence comprising an enhancer sequence and encoding a second transcription factor that controls gene expression in nascent motor neurons wherein the enhancer is capable of driving a motor-neuron specific expression of the second transcription factor, and wherein the first and second transcription factors are expressed in motor neurons of the subject.

In some embodiments, the motor neurons are rejuvenated, and/or their resistance to ALS pathogens in the subject with ALS is increased.

In some embodiments, the enhancer sequence comprises SEQ ID NO: 1. In some embodiments, the enhancer sequence consists of SEQ ID NO: 1. In some embodiments, the enhancer is ChatE.

In some embodiments, the first transcription factor is Lhx3. In some embodiments, the second transcription factor is Isl1. In some embodiments, the first transcription factor is Isl1 and the second transcription factor is Lhx3. In some embodiments, the nucleic acid sequence encoding Isl1 comprises nucleotides 3844-4890 of SEQ ID NO: 10. In some embodiments, the nucleic acid sequence encoding Isl1 comprises a nucleotide sequence encoding an amino acid sequence as encoded by nucleotides 3844-4890 of SEQ ID NO: 10. In some embodiments, the nucleic acid sequence encoding Isl1 consists of nucleotides 3844-4890 of SEQ ID NO: 10. In some embodiments, the nucleic acid sequence encoding Lhx3 comprises nucleotides 3844-5052 of SEQ ID NO: 11. In some embodiments, the nucleic acid sequence encoding Lhx3 comprises a nucleotide sequence encoding an amino acid sequence as encoded by nucleotides 3844-5052 of SEQ ID NO: 11. In some embodiments, the nucleic acid sequence encoding Lhx3 consists of nucleotides 3844-5052 of SEQ ID NO: 11. In some embodiments, the one or more transcription factors is Phox2a. In some embodiments, the one or more transcription factors is Sox2. In some embodiments, the one or more transcription factors are Phox2a and Isl1. In some embodiments, the one or more transcription factors is Oct4. In some embodiments, the one or more transcription factors are Oct4 and Sox2. In some embodiments, the nucleic acid sequence encoding Phox2a comprises a nucleotide sequence encoding an amino acid sequence of SEQ ID NO: 12. In some embodiments, the nucleic acid sequence encoding Oct4 comprises a nucleotide sequence encoding an amino acid sequence of SEQ ID NO: 13. In some embodiments, the nucleic acid sequence encoding Sox2 comprises a nucleotide sequence encoding an amino acid sequence of SEQ ID NO: 14.

In some embodiments, the AAVs of the second composition comprise a nucleic acid comprising the nucleic acids between the AAV ITRs of SEQ ID NO:10 (i.e., nucleotides 2459-5955). In some embodiments, the AAVs of the second composition comprise a nucleic acid consisting of the nucleic acids between the AAV ITRs of SEQ ID NO:10 (i.e., nucleotides 2459-5955). In some embodiments, the AAVs of the first composition comprise a nucleic acid comprising the nucleic acids between the AAV ITRs of SEQ ID NO:11 (i.e., nucleotides 2459-6120). In some embodiments, the AAVs of the first composition comprise a nucleic acid consisting of the nucleic acids between the AAV ITRs of SEQ ID NO:11 (i.e., nucleotides 2459-6120).

In some embodiments, the AAVs of the second composition comprise a nucleic acid comprising the nucleic acids between the AAV ITRs of SEQ ID NO:10 (i.e., nucleotides 2459-5955). In some embodiments, the AAVs of the second composition comprise a nucleic acid consisting of the nucleic acids between the AAV ITRs of SEQ ID NO:10 (i.e., nucleotides 2459-5955). In some embodiments, the AAVs of the first composition comprise a nucleic acid comprising the nucleic acids between the AAV ITRs of SEQ ID NO:10 (i.e., nucleotides 2459-5955), except that the CDS of Isl1 is replaced with a nucleic acid sequence encoding Phox2a. In some embodiments, the AAVs of the first composition comprise a nucleic acid consisting of the nucleic acids between the AAV ITRs of SEQ ID NO: 10 (i.e., nucleotides 2459-5955), except that the CDS of Isl1 is replaced with a nucleic acid sequence encoding Phox2a.

In some embodiments, the AAVs of the second composition comprise a nucleic acid comprising the nucleic acids between the AAV ITRs of SEQ ID NO:10 (i.e., nucleotides 2459-5955), except that the CDS of Isl1 is replaced with a nucleic acid sequence encoding Oct4. In some embodiments, the AAVs of the second composition comprise a nucleic acid consisting of the nucleic acids between the AAV ITRs of SEQ ID NO:10 (i.e., nucleotides 2459-5955), except that the CDS of Isl1 is replaced with a nucleic acid sequence encoding Oct4. In some embodiments, the AAVs of the first composition comprise a nucleic acid comprising the nucleic acids between the AAV ITRs of SEQ ID NO:10 (i.e., nucleotides 2459-5955), except that the CDS of Isl1 is replaced with a nucleic acid sequence encoding Sox2. In some embodiments, the AAVs of the first composition comprise a nucleic acid consisting of the nucleic acids between the AAV ITRs of SEQ ID NO:10 (i.e., nucleotides 2459-5955), except that the CDS of Isl1 is replaced with a nucleic acid sequence encoding Sox2.

In some embodiments, the re-expression of one or more transcription factors reactivates their embryonic targets. In some embodiments, the embryonic target MNX1.

In some embodiments, the motor neurons are spinal motor neurons.

In some embodiments, the AAVs are capable of penetrating the blood-brain barrier.

In some embodiments, administration of the AAVs attenuates disease-related proteinopathies in the motor neurons. In some embodiments, administration of the AAVs reduces the formation of p62+ aggregates in the motor neurons. In some embodiments, administration of the AAVs reduces the incidence of SQSTM1-positive round bodies. In some embodiments, administration of the AAVs reduces the formation of SOD1+ aggregates in the motor neurons. In some embodiments, administration of the AAVs results in the reduction of neuroinflammation in the vicinity of motor neurons. In some embodiments, administration of the AAVs results in the reduction of Iba1+ microglia activation in the vicinity of the motor neurons.

In some embodiments, administration of the AAVs ameliorates clinical phenotypes of ALS. In some embodiments, administration of the AAVs delays symptom onset of ALS.

In certain aspects, the present disclosure provides a composition for treating ALS in a subject in need thereof, the composition comprising AAVs, wherein the AAVs comprise a nucleic acid sequence comprising an enhancer sequence and encoding one or more transcription factors that control gene expression in nascent motor neurons, wherein the enhancer is capable of driving a motor-neuron specific expression of the one or more transcription factors and wherein the one or more transcription factors are expressed in motor neurons of the subject.

In some embodiments, the motor neurons are rejuvenated, and/or their resistance to ALS pathogens in the subject with ALS is increased.

In some embodiments, the enhancer sequence comprises SEQ ID NO: 1. In some embodiments, the enhancer sequence consists of SEQ ID NO: 1. In some embodiments, the enhancer is ChatE.

In some embodiments, the one or more transcription factors is Lhx3. In some embodiments, the one or more transcription factors is Isl1. In some embodiments, the one or more transcription factors are Isl1 and Lhx3. In some embodiments, the nucleic acid sequence encoding Isl1 comprises nucleotides 3844-4890 of SEQ ID NO: 10. In some embodiments, the nucleic acid sequence encoding Isl1 comprises a nucleotide sequence encoding an amino acid sequence as encoded by nucleotides 3844-4890 of SEQ ID NO: 10. In some embodiments, the nucleic acid sequence encoding Isl1 consists of nucleotides 3844-4890 of SEQ ID NO: 10. In some embodiments, the nucleic acid sequence encoding Lhx3 comprises nucleotides 3844-5052 of SEQ ID NO: 11. In some embodiments, the nucleic acid sequence encoding Lhx3 comprises a nucleotide sequence encoding an amino acid sequence as encoded by nucleotides 3844-5052 of SEQ ID NO: 11. In some embodiments, the nucleic acid sequence encoding Lhx3 consists of nucleotides 3844-5052 of SEQ ID NO: 11. In some embodiments, the one or more transcription factors is Phox2a. In some embodiments, the one or more transcription factors is Sox2. In some embodiments, the one or more transcription factors are Phox2a and Isl1. In some embodiments, the one or more transcription factors is Oct4. In some embodiments, the one or more transcription factors are Oct4 and Sox2. In some embodiments, the nucleic acid sequence encoding Phox2a comprises a nucleotide sequence encoding an amino acid sequence of SEQ ID NO: 12. In some embodiments, the nucleic acid sequence encoding Oct4 comprises a nucleotide sequence encoding an amino acid sequence of SEQ ID NO: 13. In some embodiments, the nucleic acid sequence encoding Sox2 comprises a nucleotide sequence encoding an amino acid sequence of SEQ ID NO: 14.

In some embodiments, the AAVs comprise a nucleic acid comprising the nucleic acids between the AAV ITRs of SEQ ID NO:10 (i.e., nucleotides 2459-5955). In some embodiments, the AAVs comprise a nucleic acid consisting of the nucleic acids between the AAV ITRs of SEQ ID NO:10 (i.e., nucleotides 2459-5955). In some embodiments, the AAVs comprise a nucleic acid comprising the nucleic acids between the AAV ITRs of SEQ ID NO:11 (i.e., nucleotides 2459-6120). In some embodiments, the AAVs comprise a nucleic acid consisting of the nucleic acids between the AAV ITRs of SEQ ID NO:11 (i.e., nucleotides 2459-6120). In some embodiments, the AAVs comprise a nucleic acid comprising the nucleic acids between the AAV ITRs of SEQ ID NO:10 (i.e., nucleotides 2459-5955), except that the CDS of Isl1 is replaced with a nucleic acid sequence encoding Phox2a. In some embodiments, the AAVs comprise a nucleic acid comprising the nucleic acids between the AAV ITRs of SEQ ID NO:10 (i.e., nucleotides 2459-5955), except that the CDS of Isl1 is replaced with a nucleic acid sequence encoding Oct4. In some embodiments, the AAVs comprise a nucleic acid comprising the nucleic acids between the AAV ITRs of SEQ ID NO:10 (i.e., nucleotides 2459-5955), except that the CDS of Isl1 is replaced with a nucleic acid sequence encoding Sox2.

In some embodiments, the motor neurons are spinal motor neurons.

In some embodiments, the AAVs are capable of penetrating the blood-brain barrier.

In certain aspects, the present disclosure provides a composition for treating ALS in a subject in need thereof, the composition comprising: a first set of AAVs and a second set of AAVs, wherein the first set of AAVs comprise a nucleic acid sequence comprising an enhancer sequence and encoding a first transcription factor that controls gene expression in nascent motor neurons and wherein the enhancer is capable of driving a motor-neuron specific expression of the first transcription factor, wherein the second set of AAVs comprise a nucleic acid sequence comprising an enhancer sequence and encoding a second transcription factor that controls gene expression in nascent motor neurons wherein the enhancer is capable of driving a motor-neuron specific expression of the second transcription factor, and wherein the first and second transcription factors are expressed in motor neurons of the subject.

In some embodiments, the motor neurons are rejuvenated, and/or their resistance to ALS pathogens in the subject with ALS is increased.

In some embodiments, the enhancer sequence comprises SEQ ID NO: 1. In some embodiments, the enhancer sequence consists of SEQ ID NO: 1. In some embodiments, the enhancer is ChatE.

In some embodiments, the first transcription factor is Lhx3. In some embodiments, the second transcription factor is Isl1. In some embodiments, the first transcription factor is Isl1 and the second transcription factor is Lhx3. In some embodiments, the nucleic acid sequence encoding Isl1 comprises nucleotides 3844-4890 of SEQ ID NO: 10. In some embodiments, the nucleic acid sequence encoding Isl1 comprises a nucleotide sequence encoding an amino acid sequence as encoded by nucleotides 3844-4890 of SEQ ID NO: 10. In some embodiments, the nucleic acid sequence encoding Isl1 consists of nucleotides 3844-4890 of SEQ ID NO: 10. In some embodiments, the nucleic acid sequence encoding Lhx3 comprises nucleotides 3844-5052 of SEQ ID NO: 11. In some embodiments, the nucleic acid sequence encoding Lhx3 comprises a nucleotide sequence encoding an amino acid sequence as encoded by nucleotides 3844-5052 of SEQ ID NO: 11. In some embodiments, the nucleic acid sequence encoding Lhx3 consists of nucleotides 3844-5052 of SEQ ID NO: 11. In some embodiments, the one or more transcription factors is Phox2a. In some embodiments, the one or more transcription factors is Sox2. In some embodiments, the one or more transcription factors are Phox2a and Isl1. In some embodiments, the one or more transcription factors is Oct4. In some embodiments, the one or more transcription factors are Oct4 and Sox2. In some embodiments, the nucleic acid sequence encoding Phox2a comprises a nucleotide sequence encoding an amino acid sequence of SEQ ID NO: 12. In some embodiments, the nucleic acid sequence encoding Oct4 comprises a nucleotide sequence encoding an amino acid sequence of SEQ ID NO: 13. In some embodiments, the nucleic acid sequence encoding Sox2 comprises a nucleotide sequence encoding an amino acid sequence of SEQ ID NO: 14.

In some embodiments, the set of AAVs comprise a nucleic acid comprising the nucleic acids between the AAV ITRs of SEQ ID NO:10 (i.e., nucleotides 2459-5955). In some embodiments, the set of AAVs comprise a nucleic acid consisting of the nucleic acids between the AAV ITRs of SEQ ID NO:10 (i.e., nucleotides 2459-5955). In some embodiments, the set of AAVs comprise a nucleic acid comprising the nucleic acids between the AAV ITRs of SEQ ID NO:11 (i.e., nucleotides 2459-6120). In some embodiments, the set of AAVs comprise a nucleic acid consisting of the nucleic acids between the AAV ITRs of SEQ ID NO:11 (i.e., nucleotides 2459-6120). In some embodiments, the set of AAVs comprise a nucleic acid comprising the nucleic acids between the AAV ITRs of SEQ ID NO:10 (i.e., nucleotides 2459-5955), except that the CDS of Isl1 is replaced with a nucleic acid sequence encoding Phox2a. In some embodiments, the set of AAVs comprise a nucleic acid comprising the nucleic acids between the AAV ITRs of SEQ ID NO:10 (i.e., nucleotides 2459-5955), except that the CDS of Isl1 is replaced with a nucleic acid sequence encoding Oct4. In some embodiments, the set of AAVs comprise a nucleic acid comprising the nucleic acids between the AAV ITRs of SEQ ID NO:10 (i.e., nucleotides 2459-5955), except that the CDS of Isl1 is replaced with a nucleic acid sequence encoding Sox2.

In some embodiments, the re-expression of one or more transcription factors reactivates their embryonic targets. In some embodiments, the embryonic target is MNX1.

In some embodiments, the motor neurons are spinal motor neurons.

In some embodiments, the AAVs are capable of penetrating the blood-brain barrier.

In certain aspects, the present disclosure provides a vector for treating ALS in a subject in need thereof, the vector comprising a nucleic acid sequence comprising an enhancer sequence and encoding one or more transcription factors that control gene expression in nascent motor neurons, wherein the enhancer is capable of driving a motor-neuron specific expression of the one or more transcription factors and wherein the one or more transcription factors are expressed in motor neurons of the subject.

In some embodiments, the motor neurons are rejuvenated, and/or their resistance to ALS pathogens in the subject with ALS is increased.

In some embodiments, the enhancer sequence comprises SEQ ID NO: 1. In some embodiments, the enhancer sequence consists of SEQ ID NO: 1. In some embodiments, the enhancer is ChatE.

In some embodiments, the one or more transcription factors is Lhx3. In some embodiments, the one or more transcription factors is Isl1. In some embodiments, the one or more transcription factors are Isl1 and Lhx3. In some embodiments, the nucleic acid sequence encoding Isl1 comprises nucleotides 3844-4890 of SEQ ID NO: 10. In some embodiments, the nucleic acid sequence encoding Isl1 comprises a nucleotide sequence encoding an amino acid sequence as encoded by nucleotides 3844-4890 of SEQ ID NO: 10. In some embodiments, the nucleic acid sequence encoding Isl1 consists of nucleotides 3844-4890 of SEQ ID NO: 10. In some embodiments, the nucleic acid sequence encoding Lhx3 comprises nucleotides 3844-5052 of SEQ ID NO: 11. In some embodiments, the nucleic acid sequence encoding Lhx3 comprises a nucleotide sequence encoding an amino acid sequence as encoded by nucleotides 3844-5052 of SEQ ID NO: 11. In some embodiments, the nucleic acid sequence encoding Lhx3 consists of nucleotides 3844-5052 of SEQ ID NO: 11. In some embodiments, the one or more transcription factors is Phox2a. In some embodiments, the one or more transcription factors is Sox2. In some embodiments, the one or more transcription factors are Phox2a and Isl1. In some embodiments, the one or more transcription factors is Oct4. In some embodiments, the one or more transcription factors are Oct4 and Sox2. In some embodiments, the nucleic acid sequence encoding Phox2a comprises a nucleotide sequence encoding an amino acid sequence of SEQ ID NO: 12. In some embodiments, the nucleic acid sequence encoding Oct4 comprises a nucleotide sequence encoding an amino acid sequence of SEQ ID NO: 13. In some embodiments, the nucleic acid sequence encoding Sox2 comprises a nucleotide sequence encoding an amino acid sequence of SEQ ID NO: 14.

In some embodiments, the vector comprises a nucleic acid comprising the nucleic acids between the AAV ITRs of SEQ ID NO:10 (i.e., nucleotides 2459-5955). In some embodiments, the vector comprises a nucleic acid consisting of the nucleic acids between the AAV ITRs of SEQ ID NO:10 (i.e., nucleotides 2459-5955). In some embodiments, the vector comprises a nucleic acid comprising the nucleic acids between the AAV ITRs of SEQ ID NO:11 (i.e., nucleotides 2459-6120). In some embodiments, the vector comprises a nucleic acid consisting of the nucleic acids between the AAV ITRs of SEQ ID NO:11 (i.e., nucleotides 2459-6120). In some embodiments, the vector comprises SEQ ID NO: 10. In some embodiments, the vector consists of SEQ ID NO: 10. In some embodiments, the vector comprises SEQ ID NO: 11. In some embodiments, the vector consists of SEQ ID NO: 11. In some embodiments, the vector comprises a nucleic acid comprising the nucleic acids between the AAV ITRs of SEQ ID NO:10 (i.e., nucleotides 2459-5955), except that the CDS of Isl1 is replaced with a nucleic acid sequence encoding Phox2a. In some embodiments, the vector comprises a nucleic acid comprising the nucleic acids between the AAV ITRs of SEQ ID NO:10 (i.e., nucleotides 2459-5955), except that the CDS of Isl1 is replaced with a nucleic acid sequence encoding Oct4. In some embodiments, the vector comprises a nucleic acid comprising the nucleic acids between the AAV ITRs of SEQ ID NO:10 (i.e., nucleotides 2459-5955), except that the CDS of Isl1 is replaced with a nucleic acid sequence encoding Sox2.

In some embodiments, the motor neurons are spinal motor neurons.

In some embodiments, the AAVs are capable of penetrating the blood-brain barrier.

### BRIEF DESCRIPTION OF FIGURES

The patent or application file contains at least one drawing executed in color. Copies of this patent or patent application publication with color drawing(s) will be provided by the Office upon request and payment of the necessary fee.

FIGS. 1A-G show the impact of re-expression of Isl1 and Lhx3 together, or either factor on their own on the incidence of p62 aggregation compared to controls at P45 (postnatal day 45). FIGS. 1A-C show that in AAV-transduced motor neurons, re-expression of Isl1 and Lhx3 together, or either factor on their own, significantly diminishes the incidence of p62 aggregation in individual motor neurons, compared to AAV-mCherry-transduced controls at P45. FIGS. 1C-D show that AAVs administered at high titer significantly impact the percentage of p62 aggregate-positive cells in the spinal cord overall, not just on a cell-by-cell basis. FIG. 1E shows correlation of overall percentage of p62 aggregate+ cells in the spinal cord with transduction efficiency. FIG. 1F-G show ectopic expression of the cranial motor neuron specifying factor Phox2a on its own and co-expression of Isl1 and Phox2a together on their impact in reducing p62 aggregation. Low titer viruses are injected at 3E+10 viral genomes/animal and high titer viruses at 3E+11 viral genomes/animal.

FIGS. 2A-B show Isl1 and Lhx3 re-expression continue to reduce the incidence of p62 aggregation at P75. FIG. 2A shows immunostaining for p62 at P75 in animals treated with low titer viruses (3E+10 viral particles/animal). FIG. 2B shows the quantification of p62+ motor neurons of FIG. 2A in treatment and control conditions.

FIGS. 3A-B show that Isl1 and Lhx3 re-expression reduce the incidence of SOD1 aggregation at P75. FIG. 3A shows immunostaining for SOD1 at P75 in animals treated with low titer (3E+10 viral particles/animal) AAV Isl1 and Lhx3. FIG. 3B shows the quantification of the percentage of motor neurons in FIG. 3A that have SOD1 aggregates in treatment and control conditions.

FIGS. 4A-B show that Isl1 and Lhx3 re-expression reduces neuroinflammation in the ventral horn of the spinal cord at P75. FIG. 4A shows immunostaining for Iba1 at P75 in animals treated with low titer (3E+10 viral particles/animal) AAV Isl1 and Lhx3. FIG. 4B shows the quantification of the mean Iba1 cell body area in treatment and control conditions.

FIGS. 5A-F show the design and in vivo validation of ChatE-driven Isl1 and Lhx3 AAVs. FIG. 5A shows a schematic of ATAC-seq data showing accessibility of ChatE (vertical rectangular box) in spinal motor neurons over time. FIG. 5B shows a design of AAV-ChatE constructs and experimental design for evaluating AAV-Isl1+AAV+Lhx3 in SOD1^{G93A} ALS model mice. FIG. 5C shows immunofluorescent staining of ectopic ISL1 and LHX3 in the L4-L5 region of the spinal cord in a P14 mouse. Ectopic ChatE-driven ISL1 and LHX3 expression is restricted to CHAT+ motor neurons in the ventral horn (first row, 5× epifluorescence), and most transduced motor neurons are double-positive for ISL1 and LHX3 (second row, 40× confocal). FIG. 5D shows distribution of ISL+, LHX3+, ISL+LHX3+, and non-transduced cells among CHAT+ cells in the L4-L5 ventral horn at P45 from animals treated with low titer (6-9E+10 vg/animal) or high titer AAVs (3-4+11 vg/animal). The mean percentage of motor neurons in each condition was quantified from 6 hemisections per animal and 15 confocal images per hemisection from 2 (low titer) or 6 (high titer) animals. Error bars represent SEM. FIG. 5E shows immunostaining for the ISL1+LHX3 target MNX1 in spinal motor neurons in the ventral horn in P45 animals. MNX1 is undetectable in untreated animals, but upregulated in ISL+ motor neurons in animals treated with AAV-Isl1+AAV-Lhx3. Images are maximum intensity projections of 15 z sections taken 3μ apart. FIG. 5F shows quantification of MNX1+ motor neurons in the L4-L5 region of the spinal cord in untreated or AAV-Isl1+AAV-Lhx3 animals at P45. The percentage of MNX1+ cells among all CHAT+ cells was quantified in 15 confocal images from 1 (untreated) or 6 (AAV-treated) 70μ hemisections per animal. Each point represents percentage per hemisection (untreated) or mean percentage per hemisection (AAV-treated) from one animal. Black bars represent animal medians per treatment condition. Significance determined by unpaired two-tailed t test (p=0.0004; t=5.164, df=10).

FIGS. 6A-E show attenuate ALS phenotypes atenuation in SOD1G93A mice with ISL1 and LHX3 re-expression. FIG. 6A shows representative immunostaining of SQSTM1 in L4-L5 ventral horn sections across treatment conditions in NTG and SOD1G93A mice at P45. Transgene images represent immunostaining for LHX3 (NTG uninjected, SOD1 uninjected and SOD1 Isl1+Lhx3) or RFP (SOD1 mCherry). Regions within dashed boxes are enlarged in Inset. Images are maximum intensity projections of 15 z sections taken 3μ apart. FIG. 6B shows quantification of SQSTM1 round bodies in AAV-transduced motor neurons in the L4-L5 region of the spinal cord in AAV-mCherry or AAV-Isl1+AAV-Lhx3-treated SOD1G93A animals. The percentage of CHAT+/mCherry+ or CHAT+/LHX3+ motor neurons exhibiting SQSTM1+ round bodies was quantified in 15 confocal sections from 6 70μ hemisections per animal. Each point represents the mean percentage per hemisection from one animal. Black bars represent animal medians per treatment condition. Significance determined by unpaired two-tailed t test (p<0.0001; t=6.290, df=23). FIG. 6C shows representative immunostaining of human SOD1 in L4-L5 ventral horn sections across treatment conditions and time. Regions within dashed boxes are enlarged in Inset. Images are maximum intensity projections of 3 z sections taken 3 μ apart. FIG. 6D shows quantification of SOD1 aggregation in AAV-transduced motor neurons in the L4-L5 region of the spinal cord in AAV-mCherry or AAV-Isl1+AAV-Lhx3-treated SOD1G93A animals at P75. The percentage of CHAT+/mCherry+ or CHAT+/LHX3+ motor neurons exhibiting SOD1 aggregates was quantified in 15 confocal sections from 6 70μ hemisections per animal. Each point represents the mean percentage per hemisection from one animal. Black bars represent animal medians per treatment condition. Significance determined by unpaired two-tailed t test (p=0.0042, t=3.800, df=9). FIG. 6E shows comparison of the probability of tremor onset in AAV-mCherry vs AAV-Isl1+AAV-Lhx3-treated SOD1G93A mice by Log-rank (Mantel-Cox) tests (n=10-13 animals per treatment per sex). Separate analyses were performed for male vs. female animals. For females, AAV-Isl1+AAV-Lhx3 treatment delayed tremor onset by 15 days (p=0.0036). For males, tremor onset was delayed by 5 days but was not significant (p=0.1138).

FIGS. 7A-E show quantification of endogenous and ectopic ISL1 and LHX3 over time. FIG. 7A shows that endogenous Isl1 and Lhx3 expression decrease sharply after their peak at E13.5 (RNAseq data from Reference 5 of Example 5; expression for each time point normalized to values at E13.5). FIG. 7B shows endogenous ISL1 and LHX3 are undetectable by immunofluorescence in CHAT+ cells in the L4-L5 ventral horn in untreated animals at P45. FIG. 7C shows schematic of the 1000 bp ChatE demonstrating the presence of transcription factor binding motifs enriched during motor neuron specification or maturation. FIG. 7D shows quantification of transduced motor neurons in L4-L5 ventral horn at P45 in animals treated with AAV-Is1 or AAV-Lhx3 (3.6E+11-3.8E+11 vg/animal) at P1. Sample size (n) was 2-4 animals per treatment per time point. Points represent mean transduced motor neurons per hemisection per animal, quantified across 6 70μ hemisections per animal from 15 confocal images per hemisection. Error bars represent SEM. FIG. 7E shows RNA-seq derived expression levels of Mnx1 in mouse motor neurons over time. Numbers on the y-axis represent expression values normalized across all genes in all samples.

FIG. 8 shows a correlation between motor neuron transduction efficiency and incidence of SQSTM1 aggregates. Animals were treated with a range of AAV-Isl1+AAV-Lhx3 doses at P1 (6.37E+10-3.69E+11 vg/animal). Quantification of SQSTM1 round bodies and ISL1 and LHX3 expression in CHAT+ motor neurons at P45 was performed in 6 70μ L4-L5 hemisections per animal from 15 confocal images per hemisection. Each point represents one animal. Correlation between the percentage of all CHAT+ motor neurons per hemisection (transduced or not) exhibiting SQSTM1 round bodies was correlated with the percentage of CHAT+ motor neurons in the same hemisections that were transduced with AAV-Isl1 and/or AAV-Lhx3 by simple linear regression (R²=0.5910; p=0.0003).

FIG. 9 shows a schematic model of transcriptional rejuvenation of spinal motor neurons.

FIG. 10 shows the SEQ ID NO: 10 and accompanying features.

FIG. 11 shows the SEQ ID NO: 11 and accompanying features.

FIGS. 12A-B show SQSTM1 aggregation in lumbar spinal motor neurons with Phox2a and Isl1 expression. FIG. 12A shows representative immunostaining of SQSTM1 in lumbar spinal motor neurons across treatment conditions (AAV-mCherry and AAV-ChatE-Phox2a+AAV-ChatE-Isl1) in SOD1G93A mice. Transgene images represent immunostaining for Phox2a. FIG. 12B shows quantification of SQSTM1 round bodies in AAV-transduced motor neurons in in lumbar spinal motor neurons in AAV-mCherry or AAV-ChatE-Phox2a+AAV-ChatE-Isl1 animals.

FIG. 13 shows representative immunostaining of Sox2 in lumbar spinal motor neurons across treatment conditions (control and AAV driving the expression of Oct4 and Sox2) in SOD1G93A mice. Staining for Sox2 picks up both AAV-driven SOX2 protein in adult motor neurons, where it is not normally expressed, as well as endogenous SOX2 in glia.

FIG. 14 shows representative immunostaining of SQSTM1 in lumbar spinal motor neurons across treatment conditions (control, AAV expressing Oct4 and Sox2). Note: staining for Sox2 picks up both AAV-driven SOX2 protein in adult motor neurons, where it is not normally expressed, as well as endogenous SOX2 in glia.

FIGS. 15A-J shows ISL1 and LHX3 re-expression drive motor neuron subtype-specific changes in gene expression. FIG. 15A shows experimental design for evaluating AAV-Isl1+AAV+Lhx3 in vivo. FIG. 15B shows immunostaining of ectopic ISL1 and LHX3 in the L4-L5 region of the spinal cord in a P14 mouse. Ectopic ChatE-driven ISL1 and LHX3 expression is restricted to CHAT+ motor neurons in the ventral horn (first row, 5× epifluorescence; scale bar represents 200 m), and most transgene-expressing motor neurons are double-positive for ISL1 and LHX3 (second row, 40× confocal; scale bar represents 20 m). FIG. 15C shows immunostaining for the ISL1+LHX3 target MNX1 in spinal motor neurons in the ventral horn in P45 animals. MNX1 is undetectable in untreated animals but is upregulated in ISL+LHX3+ (filled arrowheads) and ISL-LHX3+ (outlined arrowhead) motor neurons in animals treated with AAV-Isl1+AAV-Lhx3. Images are maximum intensity projections of 12 z sections taken 3 m apart. Scale bar represents 20 m. FIG. 15D shows quantification of MNX1+ motor neurons in the L4-L5 region of the spinal cord in untreated or AAVIsl1+AAV-Lhx3 animals at P45. The percentage of MNX1+ cells among all CHAT+ cells was quantified in 15 confocal images from 1 (untreated) or 6 (AAV-treated) 70 m hemisections per animal. Each point represents mean percentage per hemisection from one animal; triangles represent males, and circles represent females. Black bars represent animal medians per treatment condition. Significance determined by unpaired two-tailed t test (p=0.0004, t=5.164, df=10). FIG. 15E shows clustering of single nuclei multiome dataset using Canonical Correlation Analysis (CCA)-based integration of snRNA-seq data. Clusters are labeled based on expression of Chat (C=cholinergic), Slc17ac6 (E=excitatory), Gad1 (I=inhibitory), or >1 of these three genes (M=multiple). The three motor neurons clusters are labeled Alpha, Gamma, or Type 3 based on known subtype markers. FIG. 15F shows that each cluster is populated by both control and treatment cells. FIG. 15G shows Left: Normalized ATAC signal (range 0-4700) at the ChatE in all clusters. High signal is apparent in C1:Gamma motor neurons, C2: Type 3 motor neurons, C5: Alpha motor neurons and cluster M5.n Right: Violin plots showing expression of WPRE transcripts from the AAV. The highest levels of expression are seen in alpha and type 3 motor neurons and in cluster M5. FIG. 15H shows re-clustering of C1: Gamma, C2: Type 3, and C5: Alpha motor neurons clusters from (A) without the use of integration anchors. WPRE expression is highest in clusters 6 and 8. FIGS. 15I-J show treatment gamma cells are found in the same clusters with control gamma cells. However, most of the treatment alpha and type3 cells form independent clusters (alpha prime and type 3 prime, respectively), separate from clusters populated largely by control alpha and type 3 cells.

FIGS. 16A-G shows gene expression changes in unique alpha and type 3 clusters are consistent with more youthful state. FIG. 16A shows plots of DEGs with log 2 fold-change of at least 0.25 and an adjusted p value <0.05 for alpha motor neuron-specific cluster 8 compared to clusters 2, 9, and 10 (blue), and for type 3 motor neuron-specific cluster 6 compared to clusters 0 and 7 (pink). Each point represents one gene. FIG. 16B shows representative immunostaining for ISL1 (red) and CADPS2 (green) in AAV-mCherry and AAVIsl1+Lhx3-treated animals. AAV-Isl1+AAV-Lhx3 treatment leads to up-regulation of CADPS2 in AAV+ cells. Images are maximum intensity projections of 13 z sections taken 3 m apart. Scale bar represents 20 m. FIG. 16C shows quantification of CADPS2+ nuclei per hemisection. Each point represents data from one hemisection, with 6 hemisections assessed per animal, n=4 (AAVmCherry) or n=5 (AAV-Isl1+Lhx3) animals per treatment condition. Black bars represent median values. Significance determined by unpaired two-tailed t test (CADPS2+ nuclei p<0.0001, t=5.479, df=39; CADPS2 intensity p<0.0001, t=4.532, df=52). FIG. 16D shows heatmaps of ATAC-seq reads at genomic regions that gain accessibility only in treatment alpha clusters (top), only in treatment type 3 clusters (bottom) or in both treatment alpha and type 3 clusters (middle). FIG. 16E shows the top 2 motifs among upregulated peaks are the LHX3 binding motif and a related homeodomain motif. FIG. 16F shows alpha and type 3 DEGs from (A) plotted to show the timepoint at which they reach maximal expression during normal development based the longitudinal bulk sequencing dataset from 2. Expression values for each gene are normalized so that minimum value between E13.5 and P21 is represented as 0, and maximum value is 1. FIG. 16G shows correlations for the alpha and type 3 DEGs between AAV-Isl1-Lhx3-mediated changes in gene expression (from a) vs. maturation-mediated changes in expression between P21 and P4 (from 2). For alpha DEGs, R2=0.4963, p<0.0001. For type 3 DEGs, R2=0.07469, p<0.0001.

FIGS. 17A-C shows ISL1 and LHX3 re-expression attenuates motor neuron pathologies in SOD1G93A mice. FIG. 17A shows Quantification of SQSTM1 round bodies in transgene-expressing motor neurons in the L4-L5 region of the spinal cord in AAV-mCherry or AAV-Isl1+AAV-Lhx3-treated SOD1G93A animals. The percentage of CHAT+/mCherry+ or CHAT+/LHX3+ motor neurons exhibiting SQSTM1 round bodies was quantified in 15 confocal sections from 6 70 μm hemisections per animal. Each point represents the mean percentage per hemisection from one animal; triangles represent males, and circles represent females. Black bars represent animal medians per treatment condition. Significance determined by unpaired two-tailed t test (p<0.0001; t=6.290, df=23). FIG. 17B shows Representative immunostaining of human SOD1 in L4-L5 ventral horn sections across treatment conditions and time. Regions within dashed yellow boxes are enlarged in Inset. Images are maximum intensity projections of 3 z sections taken 3 m apart. Scale bar represents 20 μm. FIG. 17C shows Quantification of SOD1 pathology in transgene-expressing motor neurons in the L4-L5 region of the spinal cord in AAVmCherry or AAV-Isl1+AAV-Lhx3-treated SOD1G93A animals at P75. The percentage of CHAT+/mCherry+ or CHAT+/LHX3+ motor neurons exhibiting SOD1 pathology was quantified in 15 confocal sections from 6 70 μm hemisections per animal. Each point represents the mean percentage per hemisection from one animal. Significance determined by unpaired two-tailed t test (p=0.0001, t=5.669, df=12).

FIGS. 18A-E shows ISL1 and LHX3 re-expression attenuates MN degeneration in SOD1G93A mice. FIG. 18A shows Representative immunostaining of motor neuron survival and transgene expression in L4-L5 ventral horn sections across treatment conditions in SOD1G93A mice at P120. Images are maximum intensity projections of 11 z sections taken 3 m apart. Scale bar represents 20 m. FIG. 18B shows Quantification of total CHAT+ motor neurons per hemisection in the L4-L5 region of the spinal cord in AAV-mCherry or AAV-Isl1+AAVLhx3− treated SOD1G93A animals at P120 in 15 confocal sections from 12 70 μm hemisections per animal. Each point represents the mean motor neuron count per hemisection from one animal; triangles represent males, and circles represent females. Black bars represent animal medians per treatment condition. Significance determined by unpaired two-tailed t test (p=0.0024, t=3.692, df=14). FIG. 18C shows Percentage of transgene-expressing cells among all CHAT+ motor neurons from b. Significance determined by two way ANOVA for the effects of treatment (DF=1, F(1,12)=1.197, p=0.2954) and sex (DF=1, F(1,12)=0.006892, p=0.9352) with Dunnett's multiple comparisons test. FIGS. 18D-E show distribution of transgene expression among CHAT+ motor neurons in AAVIsl1+ AAV-Lhx3-treated SOD1G93A animals at P45 (high-titer-treated animals from Supplemental FIG. 3a) and P120 (from b). Error bars represent SEM.

FIGS. 19A-C show transfection efficiency of ChatE constructs. FIGS. 19A-19B show Quantification and representative images of mCherry distribution across spinal cord and dorsal root ganglia (DRG) sections from n=3 animals injected with low titer AAVmCherry (3.19E+10 vg/animal) at P1 and analyzed at P45. In FIG. 19A, tissues from the same animal were used to quantify the incidence of mCherry+ cells across the indicated cell types. Circles represent females; triangle represents male. In FIG. 19B, whole cord images were taken at 5× on an epifluorescence microscope (scale bar=200 m). Unfilled arrowhead indicates V0c interneurons; dashed lines indicate PGCs represented in inset. PGC inset images were taken at 40× on a confocal microscope are maximum projections. of 7 z sections taken 3 m apart (scale bar=20 μm). DRG images were taken at 20× on an epifluorescence microscope (scale bar=200 μm). Filled arrowheads indicate mCherry+ sensory neuron. FIG. 19C shows representative 5× epifluorescence images of whole L4-L5 spinal cord sections from animals injected at P1 with low titer AAV-NLS-GFP (2.10E+10 vg/animal). Dashed yellow line indicates region shown in inset.

FIGS. 20A-B shows distribution of ISL+, LHX3+, ISL+LHX3+, and ISL1−LHX3− cells among CHAT+ cells in the L4-L5 ventral horn at P45. FIG. 20A shows distribution of AAV+ motor neurons among the high titer animals plotted in FIG. 7E, separated by sex (n=3 animals per sex). No significant effect of sex was observed by one-way ANOVA (p=0.3739). FIG. 20B shows quantification of total CHAT+ motor neurons per hemisection in the L4-L5 region of the spinal cord in untreated animals or in AAV-mCherry-treated or AAV-Isl1+AAV-Lhx3-treated injected at P1 and analyzed at P45 in 15 confocal sections from 6 70 μm hemisections per animal. Each point represents the mean motor neuron count per hemisection from one animal; triangles represent males, and circles represent females. Black bars represent animal medians per treatment condition. No significant effect of treatment on motor neuron survival was observed by one-way ANOVA (p=0.1600).

FIGS. 21A-E shows differential expression of markers between control and treatment mice. FIG. 21A shows percent control and treatment cells in all clusters. While there is some variation between clusters, all clusters are comprised of both control and treatment cells. FIG. 21B shows Violin plot showing expression of the neuronal gene Snap25, the cholinergic gene Chat, the excitatory gene Slc17a6, and the inhibitory gene Gad1 in clusters from FIG. 16C. FIG. 21B shows dot plot showing expression levels and percent cells expressing known alpha, gamma, and type 3 markers in cholinergic clusters. FIG. 21D shows violin plot showing distribution of the four AAV elements across CCA clusters. FIG. 21E shows Differential gene expression between AAV transcript+ treatment cells vs. control cells in motor neurons and M5 clusters. Gene expression changes are largely restricted to alpha and type 3 motor neurons.

FIGS. 22A-B shows Representative immunostaining in the L4-L5 region of the ventral horn from animals injected with AAV-Isl1+AAV-Lhx3 (6.38E+10 total vg/animal) at P1 and analyzed at P14 (FIG. 22A) or P21 (FIG. 22B). Images are maximum intensity projections of 8 z sections taken 3 m apart. Filled arrowheads indicate ectopic ISL1 and/or LHX3-expressing gamma motor neurons, identified as small-diameter CHAT+ cells that lack NEUN expression. Outlined arrowheads indicate gamma motor neurons that do not currently express ISL1 or LHX3. Scale bars represent 20 μM.

FIGS. 23A-F shows RNA-sequencing analysis of motor neurons. FIG. 23A shows expression of alpha, gamma, and type 3 markers in motor neuron-specific clusters. FIG. 23B shows percent control and treatment cells in motor neuron-specific clusters. Two of the clusters, 6 and 8 (termed type 3 prime and alpha prime, respectively), are composed solely of treatment cells. These clusters represent type 3 (6) and alpha (8) motor neurons from the treatment group. FIG. 23C shows that Cluster 4 is similar to one found in published single nuclei RNAseq of adult motor neurons (publicly accessible at spinalcoratlas.org) that is found near alpha cells but lacks key marker that label most alpha cells (Stk32a, Sv2b). The 7 markers shown here to be expressed in cluster 4 are also expressed in the corresponding cluster in the published data. FIG. 23D shows Percent control and treatment cells in non-motor neuron cholinergic clusters. While there is some variation between clusters, all clusters have both control and treatment cells. FIGS. 23E-F show clustering of non-motor neuron cholinergic cells, and composition of control and treatment nuclei in each cluster.

FIGS. 24A-E shows quantification of differential gene expression. FIGS. 24A-B shows dotplots showing log 2 fold expression change in alpha prime vs. alpha clusters (alpha DEGs) or type 3 prime vs. type 3 clusters on x-axis and log 2 fold expression change in the same genes in treated bulk RNAseq data vs. control bulk RNAseq data. There is an overall positive correlation in these gene expression changes despite differences in the methodology and sensitivity of snRNAseq vs. bulk RNAseq. The positive correlation with bulk RNAseq is stronger for alpha than type 3 DEGs. FIG. 24C shows representative immunostaining for RFP in the AAV-mCherry-treated animal included in FIG. 15B showing extensive transgene expression in CHAT+ motor neurons. Scale bar represents 20 μm. FIG. 24D shows enlarged regions of images from FIG. 16B showing MNX1 expression in a subset of ISL1+ cells that show strong nuclear CADPS2 staining (arrowheads). No MNX1 signal was detected in animals injected with control mCherry virus. Scale bar represents 20 m. FIG. 24E shows UMAP from FIG. 15H showing distribution and prevalence of Lhx3 motifs among differentially accessible peaks in each cluster.

FIGS. 25A-B shows gene expression profiles of alpha and type 3 DEGS treated with AAV-Isl1-Lhx3. FIG. 25A shows a heatmap of R2 values for correlations between AAV-Isl1-Lhx3-mediated changes in gene expression vs. maturation-mediated changes in expression between P21 and the indicated time points for alpha and type 3 DEGs. Values for P4 are reported in FIG. 16G. p values for alpha DEGs are as follows: E13.5, p<0.0001; P4, p<0.0001; P13, p<0.0001. p values for type 3 DEGs are as follows: E13.5, p<0.0001; P4, p<0.0001; P13, p=0.0002. Minimum R2 value=0.03823; maximum R2 value=0.4063. FIG. 25B shows a bubble plot of FDR values for shared, significantly enriched GO terms between up-regulated alpha and type 3 DEGs. Maximum FDR value=0.0381; minimum FDR value=8.09E-12. Bubble size represents the percentage of shared DEGs populating each term.

FIG. 26A-F shows functional differences in motor neurons treated with AAV-Isl1+AAV-Lhx3. FIG. 26A shows correlation between motor neuron transduction efficiency and incidence of SQSTM1 aggregates. Animals were treated with a range of AAV-Isl1+AAV-Lhx3 doses at P1 (6.37E+10-3.69E+11 vg/animal). Quantification of SQSTM1 round bodies and ISL1 and LHX3 expression in CHAT+ motor neurons at P45 was performed in 6 70 μm L4-L5 hemisections per animal from 15 confocal images per hemisection. Each point represents one animal. The percentage of all CHAT+ motor neurons per hemisection (whether positive for ISL1 or LHX3 or not) that exhibited SQSTM1 round bodies was correlated with the percentage of CHAT+ motor neurons in the same hemisections that were positive for ectopic ISL1 and/or LHX3 expression by simple linear regression, segregated by sex (Males: R squared=0.5745; p=0.0180; Females: R squared=0.7023, p=0.0094). FIG. 26B shows distribution of transgene-expressing CHAT+ motor neurons among cells positive or negative for SQSTM1 round bodies from the high titer AAV-Isl1+AAV-Lhx3-treated animals included in FIG. 6B (n=6). FIG. 26C ATAC-seq reads at the genomic locus of Sqstm1 on the left, and violin plots showing gene expression on the right. Expression of Sqstm1 is not significantly different between treatment-specific clusters and control clusters. FIG. 26D shows representative immunostaining of L4-L5 ventral horn motor neurons from animals treated with high titer AAV-Isl1-Lhx3 fusion (1.80E+11 vg/animal) at P1 and analyzed at P14. CHAT+ motor neurons show strong and specific expression of both transgenes at this time point. Scale bar represents 20 m. FIG. 26E shows qPCR quantification of Isl1-Lhx3 fusion, mCherry, and human SOD1 transgene expression in whole spinal cord lysates from SOD1G93A animals injected at P1 with AAV-mCherry, AAV-Isl1-Lhx3 fusion, or left untreated, and analyzed at P14. Isl1-Lhx3 and mCherry show strong and specific expression in AAV-Isl1-Lhx3 fusion or AAV-mCherry-treated animals, respectively. Expression of the human SOD1 transgene is unchanged under all treatment conditions. Significance was determined two-way ANOVA for the effects of AAV treatment and transgene expression with Dunnett's multiple comparisons test. ****p<0.0001. Error bars represent SEM. FIG. 26F shows ATAC-seq reads at the genomic locus of endogenous Sod1 on the left, and violin plots showing gene expression on the right. Expression of endogenous Sod1 is not significantly different between treatment-specific clusters and control clusters.

FIGS. 27A-D show long term in vivo efficacy of AAV treatment. FIG. 27A shows the probability of remaining asymptomatic as a measure of tremor onset in AAV-mCherry vs AAVIsl1+AAV-Lhx3-treated SOD1G93A mice was compared by Log-rank (Mantel-Cox) tests (n=10-13 animals per treatment per sex). Separate analyses were performed for male vs. female animals. For females, AAV-Isl1+AAV-Lhx3 treatment delayed tremor onset by 15 days (p=0.0036). For males, tremor onset was delayed by 5 days but was not significant (p=0.1138). FIG. 27B shows the probability of survival in the same animals as (a) compared by Log-rank (Mantel-Cox) test. Mean survival was as follows: AAV-mCherry males=161 days; AAV-Isl1+AAV-Lhx3 males=160.5 days; AAV-mCherry females=166 days; AAVIsl1+AAV-Lhx3 females=169 days. No significant effects of treatment on survival were observed for females (p=0.8807) or males (p=0.2687). FIG. 27C shows representative immunostaining for CHAT+ motor neuron survival and transgene expression in a subset of animals from a,b collected at endstage. mCherry expression generally remains strong, while ISL1 and LHX3 expression are sparse or absent. FIG. 27D shows, in the subset of AAV-Isl1+AAV-Lhx3-treated animals analyzed at endstage, there was a trend toward a significant correlation between the percentage of CHAT+ motor neurons per hemisection expressing ISL1 or LHX3 and the number of surviving motor neurons per hemisection in the L4-L5 region of the spinal cord (R2=0.6200, p=0.0630). 6 70 μm hemisections were quantified per animal. Circles represent females; triangles represent males.

### DETAILED DESCRIPTION

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

As used herein, the term “subject” refers to a vertebrate animal. In one embodiment, the subject is a mammal or a mammalian species. In one embodiment, the subject is a human. In one embodiment, the subject is a healthy human adult. In other embodiments, the subject is a non-human vertebrate animal, including, without limitation, non-human primates, laboratory animals, livestock, racehorses, domesticated animals, and non-domesticated animals. In one embodiment, the term “human subjects” means a population of healthy human adults.

The terms “treatment,” “treating,” “treat,” “therapy,” “therapeutic,” and the like are used herein to refer generally to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a subject, and includes: (a) preventing the disease or symptom from occurring in a subject which may be predisposed to the disease or symptom, may or may not be diagnosed as having it; (b) inhibiting the disease symptom, i.e., arresting its development; or (c) relieving the disease symptom, i.e., causing regression of the disease or symptom. The expression “therapeutically effective amount” refers to an amount of an agent disclosed herein, that is effective for preventing, ameliorating, treating or delaying the onset of a disease or condition.

In certain aspects, the present disclosure provides a method for treating ALS in a subject in need thereof by re-expressing one or more transcription factors (e.g., Lhx3 and/or Isl1) that control gene expression in nascent motor neurons. In some embodiments, the method comprises administering to the subject AAVs containing enhancer-driven transcription factors to induce expression of the transcription factors in motor neurons. In some embodiments, the motor neurons are rejuvenated, and/or their resistance to ALS pathogens in a subject with ALS is increased.

In some embodiments, the enhancer is ChAT-E. In some embodiments, the one or more transcription factors is Lhx3, Isl1, Phox2a, Oct4, or Sox2. In another embodiment, the one or more transcription factors are Isl1 and Lhx3. In another embodiment, the one or more transcription factors are Isl1 and Phox2a. In another embodiment, the one or more transcription factors are Oct4 and Sox2.

In some embodiments, the ChAT-E-Isl1, ChAT-E-Lhx3, ChAT-E-Phox2a, ChAT-E-Sox2, ChAT-E-Oct4 or combinations thereof are packaged into AAVs that are blood-brain barrier penetrable.

In some embodiments, administration of the AAVs containing the enhancer-driven transcription factors results in the reduction of p62+ aggregates in motor neurons. In some embodiments, administration of the AAVs containing the enhancer-driven transcription factors results in the reduction of mutant SOD1+ aggregates.

In some embodiments, administration of the AAVs containing the enhancer-driven transcription factors results in the reduction of the cell body area of Iba1+ microglia in the vicinity of the motor neurons.

The pharmaceutical compositions of the inventions can be administered to any animal that can experience the beneficial effects of the agents of the invention. Such animals include humans and non-humans such as primates, pets and farm animals.

The present invention also comprises pharmaceutical compositions comprising the agents disclosed herein. Routes of administration and dosages of effective amounts of the pharmaceutical compositions comprising the agents are also disclosed. The agents of the present invention can be administered in combination with other pharmaceutical agents in a variety of protocols for effective treatment of disease.

Pharmaceutical compositions of the present invention are administered to a subject in a manner known in the art. The dosage administered will be dependent upon the age, health, and weight of the recipient, kind of concurrent treatment, if any, frequency of treatment, and the nature of the effect desired. One may administer the viral vectors, or related compound or composition into the cerebrospinal fluid, for example via intrathecal delivery, or by direct targeted injection into the spinal cord.

One of ordinary skill in the art will appreciate that a method of administering pharmaceutically effective amounts of the pharmaceutical compositions of the invention to a patient in need thereof, can be determined empirically, or by standards currently recognized in the medical arts. The agents can be administered to a patient as pharmaceutical compositions in combination with one or more pharmaceutically acceptable excipients. It will be understood that, when administered to a human patient, the total daily usage of the agents of the pharmaceutical compositions of the present invention will be decided within the scope of sound medical judgment by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, gender and diet of the patient; the time of administration, route of administration, and rate of excretion of the agent; the duration of the treatment; drugs used in combination or coincidental with the specific agent; and like factors well known in the medical arts. It is well within the skill of the art to start doses of the agents at levels lower than those required to achieve the desired therapeutic effect and to gradually increase the dosages until the desired effect is achieved.

Dosaging can also be administered in a patient-specific manner to provide a predetermined concentration of the agents in the blood, as determined by techniques accepted and routine in the art.

### Compositions for Treating Amyotrophic Lateral Sclerosis (ALS)

In various embodiments, the present application discloses compositions for treating ALS. In various embodiments, the present application discloses a composition that induces a motor-neuron-specific expression of one or more transcription factors. The transcription factors' expression may be induced using any known method in the art. For example, in various embodiments, the composition is a vector encoding a gene for expressing Lhx3, Isl1, Phox2a, Oct4, or Sox2. For example, in various embodiments, the composition is a vector encoding a gene for expressing Isl1 and Lhx3. For example, in various embodiments, the composition is a vector encoding a gene for expressing Isl1 and Phox2a. For example, in various embodiments, the composition is a vector encoding a gene for expressing Oct 4 and Sox2. In various embodiments, the vector is a viral vector. In various embodiments, the viral vector is an AAV vector. In various embodiments, the viral vector is a vector that preferentially targets motor neurons. In various embodiments, the AAV is AAV 1, 2, 3, 4, 5, 6, 7, 8, 9, or 10. In various embodiments, the AAV is AAV2. In various embodiments, the AAV is an AAV variant with tropism for motor neurons. In various embodiments, the AAV is an AAV variant with high blood-brain barrier penetrance. In various embodiments, the present application discloses AAVs that comprise a nucleic acid sequence with an enhancer sequence and one or more transcription factors that control gene expression in nascent motor neurons. In various embodiments, the enhancer is a ChAT enhancer. In various embodiments the enhancer comprises SEQ ID NO: 1. In some embodiments, the AAVs comprise a nucleic acid comprising the nucleic acids between the AAV ITRs of SEQ ID NO:10 (i.e., nucleotides 2459-5955). In some embodiments, the AAVs comprise a nucleic acid consisting of the nucleic acids between the AAV ITRs of SEQ ID NO:10 (i.e., nucleotides 2459-5955). In some embodiments, the AAVs comprise a nucleic acid comprising the nucleic acids between the AAV ITRs of SEQ ID NO:11 (i.e., nucleotides 2459-6120). In some embodiments, the AAVs comprise a nucleic acid consisting of the nucleic acids between the AAV ITRs of SEQ ID NO:11 (i.e., nucleotides 2459-6120). In some embodiments, the AAVs comprise a nucleic acid comprising the nucleic acids between the AAV ITRs of SEQ ID NO:10 (i.e., nucleotides 2459-5955), except that the CDS of Isl1 is replaced with a nucleic acid sequence encoding Phox2a. In some embodiments, the AAVs comprise a nucleic acid comprising the nucleic acids between the AAV ITRs of SEQ ID NO:10 (i.e., nucleotides 2459-5955), except that the CDS of Isl1 is replaced with a nucleic acid sequence encoding Oct4. In some embodiments, the AAVs comprise a nucleic acid comprising the nucleic acids between the AAV ITRs of SEQ ID NO:10 (i.e., nucleotides 2459-5955), except that the CDS of Isl1 is replaced with a nucleic acid sequence encoding Sox2. In some embodiments, a transcription factors that control gene expression in nascent motor neurons is Isl1. In some embodiments, a transcription factors that control gene expression in nascent motor neurons is Lhx3. In some embodiments, the AAV comprises a nucleic acid sequence of a ChAT enhancer and encoding Isl1. In some embodiments, the AAV comprises a nucleic acid sequence of a ChAT enhancer and encoding Lhx3. In some embodiments, the AAV comprises a nucleic acid sequence of a ChAT enhancer and encoding Lhx3 and a ChAT enhancer and encoding Isl1. In some embodiments, the nucleic acid sequence encoding Isl1 comprises nucleotides 3844-4890 of SEQ ID NO: 10. In some embodiments, the nucleic acid sequence encoding Isl1 comprises a nucleotide sequence encoding an amino acid sequence as encoded by nucleotides 3844-4890 of SEQ ID NO: 10. In some embodiments, the nucleic acid sequence encoding Isl1 consists of nucleotides 3844-4890 of SEQ ID NO: 10. In some embodiments, the nucleic acid sequence encoding Lhx3 comprises nucleotides 3844-5052 of SEQ ID NO: 11. In some embodiments, the nucleic acid sequence encoding Lhx3 comprises a nucleotide sequence encoding an amino acid sequence as encoded by nucleotides 3844-5052 of SEQ ID NO: 11. In some embodiments, the nucleic acid sequence encoding Lhx3 consists of nucleotides 3844-5052 of SEQ ID NO: 11. In some embodiments, a transcription factors that control gene expression in nascent motor neurons is Phox2a. In some embodiments, a transcription factors that control gene expression in nascent motor neurons is Oct4. In some embodiments, a transcription factors that control gene expression in nascent motor neurons is Sox2. In some embodiments, the AAV comprises a nucleic acid sequence of a ChAT enhancer and encoding Phox2a. In some embodiments, the AAV comprises a nucleic acid sequence of a ChAT enhancer and encoding Oct4. In some embodiments, the AAV comprises a nucleic acid sequence of a ChAT enhancer and encoding Sox2. In some embodiments, the AAV comprises a nucleic acid sequence of a ChAT enhancer and encoding Phox2a and a ChAT enhancer and encoding Isl1. In some embodiments, the AAV comprises a nucleic acid sequence of a ChAT enhancer and encoding Oct4 and a ChAT enhancer and encoding Sox2. In some embodiments, the nucleic acid sequence encoding Phox2a comprises a nucleotide sequence encoding an amino acid sequence of SEQ ID NO: 12. In some embodiments, the nucleic acid sequence encoding Oct4 comprises a nucleotide sequence encoding an amino acid sequence of SEQ ID NO: 13. In some embodiments, the nucleic acid sequence encoding Sox2 comprises a nucleotide sequence encoding an amino acid sequence of SEQ ID NO: 14.

In certain aspects, the invention provides a nucleic acid vector comprising: an enhancer sequence comprising a nucleotide sequence at least 70% identical to SEQ ID NO: 1; and a nucleotide sequence encoding a polypeptide of interest or an oligonucleotide of interest (e.g., a transcription factor that controls gene expression in nascent motor neurons such as Lhx3, Isl1, Phox2a, Oct4, or Sox2), wherein the nucleotide encoding the polypeptide of interest or oligonucleotide of interest is positioned 3′ to the enhancer sequence. In certain embodiments, the nucleic acid vector comprises the nucleic acid features described below. See section titled “Nucleic Acids”.

In certain embodiments, the vector is a viral vector. In some embodiments, the vector is circular, and in other embodiments, the vector is linearized. Viral vectors include adeno-associated viral (AAV), adenoviral, lentiviral, and retroviral vectors. AAVs can infect terminally differentiated cells, establish nuclear episomes without risking insertional mutagenesis, and convey long-term transgene expression and mild immune responses, making them a preferred choice of delivery to neurons. In certain embodiments, an AAV vector may convey transgene expression for at least one week, at least one month, at least four months, at least six months, at least one year, or longer.

The compositions or treatments described herein can be administered to the subject once (e.g., as a single injection or deposition). Alternatively, compositions or treatments can be administered once or twice daily to a subject in need thereof for a period of from about two to about twenty-eight days, or from about seven to about ten days. A composition or treatment can also be administered once or twice daily to a subject for a period of 1, 2, 3, 4, 5, 6, 7, 8, 9, 10, 11, 12 times per year, or a combination thereof. Furthermore, compositions or treatments can be co-administrated with another therapeutic.

The compositions or treatments of described herein can be formulated and administered to reduce the symptoms associated with ALS. Compositions or treatments can be administered by any conventional means available for use in conjunction with pharmaceuticals. Compositions or treatments can be administered alone, but are generally administered with a pharmaceutical carrier selected on the basis of the chosen route of administration and standard pharmaceutical practice.

A therapeutically effective treatment can depend upon a number of factors known to those or ordinary skill in the art. The dose(s) of a treatment can vary, for example, depending upon the identity, size, and condition of the subject or sample being treated, further depending upon the route by which the treatment is to be administered. These amounts can be readily determined by a skilled artisan. Any of the therapeutic applications described herein can be applied to any subject in need of such therapy, including, for example, a mammal such as a dog, a cat, a cow, a horse, a rabbit, a monkey, a pig, a sheep, a goat, or a human.

Pharmaceutical compositions for use in accordance with the invention can be formulated in conventional manner using one or more physiologically acceptable carriers or excipients. The therapeutic compositions of the invention can be formulated for a variety of routes of administration, including systemic and topical or localized administration. Techniques and formulations generally can be found in Remmington's Pharmaceutical Sciences, Meade Publishing Co., Easton, Pa (23rd Ed., 2020), the entire disclosure of which is herein incorporated by reference. For systemic administration, an injection is useful, including intramuscular, intravenous, intraperitoneal, intrathecal, and subcutaneous. For injection, the therapeutic compositions of the invention can be formulated in liquid solutions, for example in physiologically compatible buffers such as Hank's solution or Ringer's solution. In addition, the therapeutic compositions can be formulated in solid form and redissolved or suspended immediately prior to use. Lyophilized forms are also included. Pharmaceutical compositions of the present invention are characterized as being at least sterile and pyrogen-free. These pharmaceutical formulations include formulations for human and veterinary use.

According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation or ingestion), transdermal (topical), transmucosal, and rectal administration. Solutions or suspensions used for parenteral, intramuscular, intrathecal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the vector (e.g., AAVs with the polypeptides of interest) of the invention in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

### Nucleic Acids

In certain aspects, described herein is a nucleic acid comprising: an enhancer sequence comprising a nucleotide sequence at least 70% identical to SEQ ID NO: 1; and a nucleotide sequence encoding a polypeptide of interest or an oligonucleotide of interest (e.g., a transcription factor that controls gene expression in nascent motor neurons such as Lhx3, Isl1, Phox2a, Oct4, or Sox2), wherein the nucleotide encoding the polypeptide of interest or oligonucleotide of interest is positioned 3′ to the enhancer sequence.

In certain aspects the invention provides an expression cassette comprising a nucleic acid comprising: an enhancer sequence comprising a nucleotide sequence at least 70% identical to SEQ ID NO: 1; and a nucleotide sequence encoding a polypeptide of interest or an oligonucleotide of interest (e.g., a transcription factor that controls gene expression in nascent motor neurons such as Lhx3, Isl1, Phox2a, Oct4, or Sox2), wherein the nucleotide encoding the polypeptide of interest or oligonucleotide of interest is positioned 3′ to the enhancer sequence.

In certain aspects, the invention is directed to a nucleic acid sequence comprising SEQ ID NO: 1. In certain aspects, the invention is directed to a nucleic acid sequence consisting of SEQ ID NO: 1.

In certain aspects, the invention is directed to nucleic acid sequence variants of SEQ ID NO: 1. Variants of SEQ ID NO: 1 include, but are not limited to, nucleic acid sequences having at least from about 50% to about 55% identity to that of SEQ ID NO: 1. Variants of SEQ ID NO: 1 include, but are not limited to, nucleic acid sequences having at least from about 55.1% to about 60% identity to that of SEQ ID NO: 1. Variants of SEQ ID NO: 1 include, but are not limited to, nucleic acid sequences having at least from about 60.1% to about 65% identity to that of SEQ ID NO: 1. Variants of SEQ ID NO: 1 include, but are not limited to, nucleic acid sequences having at least from about 65.1% to about 70% identity to that of SEQ ID NO: 1. Variants of SEQ ID NO: 1 include, but are not limited to, nucleic acid sequences having at least from about 70.1% to about 75% identity to that of SEQ ID NO: 1. Variants of SEQ ID NO: 1 include, but are not limited to, nucleic acid sequences having at least from about 75.1% to about 80% identity to that of SEQ ID NO: 1. Variants of SEQ ID NO: 1 include, but are not limited to, nucleic acid sequences having at least from about 80.1% to about 85% identity to that of SEQ ID NO: 1. Variants of SEQ ID NO: 1 include, but are not limited to, nucleic acid sequences having at least from about 85.1% to about 90% identity to that of SEQ ID NO: 1. Variants of SEQ ID NO: 1, but are not limited to, nucleic acid sequences having at least from about 90.1% to about 95% identity to that of SEQ ID NO: 1. Variants of SEQ ID NO: 1 include, but are not limited to, nucleic acid sequences having at least from about 95.1% to about 97% identity to that of SEQ ID NO: 1. Variants of SEQ ID NO: 1 include, but are not limited to, nucleic acid sequences having at least from about 97.1% to about 99% identity to that of SEQ ID NO: 1.

In some embodiments, any of the enhancer sequences and their variants described herein are using in combination with a nucleic acid sequence encoding Lhx3, Isl1, Phox2a, Oct4, or Sox2. In some embodiments, the nucleic acid sequence encoding Isl1 comprises nucleotides 3844-4890 of SEQ ID NO: 10, or nucleic acid sequences having at least 70, 75, 80, 85, 90, 95, 96, 97, 98, or 99% identity. In some embodiments, the nucleic acid sequence encoding Isl1 comprises a nucleotide sequence encoding an amino acid sequence as encoded by nucleotides 3844-4890 of SEQ ID NO: 10, or an amino acid sequence having at least 70, 75, 80, 85, 90, 95, 96, 97, 98, or 99% identity. In some embodiments, the nucleic acid sequence encoding Isl1 consists of nucleotides 3844-4890 of SEQ ID NO: 10. In some embodiments, the nucleic acid sequence encoding Lhx3 comprises nucleotides 3844-5052 of SEQ ID NO: 11, or nucleic acid sequences having at least 70, 75, 80, 85, 90, 95, 96, 97, 98, or 99% identity. In some embodiments, the nucleic acid sequence encoding Lhx3 comprises a nucleotide sequence encoding an amino acid sequence as encoded by nucleotides 3844-5052 of SEQ ID NO: 11, or an amino acid sequence having at least 70, 75, 80, 85, 90, 95, 96, 97, 98, or 99% identity. In some embodiments, the nucleic acid sequence encoding Lhx3 consists of nucleotides 3844-5052 of SEQ ID NO: 11. In some embodiments, the nucleic acid sequence encoding Phox2a comprises a nucleotide sequence encoding an amino acid sequence of SEQ ID NO: 12, or an amino acid sequence having at least 70, 75, 80, 85, 90, 95, 96, 97, 98, or 99% identity. In some embodiments, the nucleic acid sequence encoding Oct4 comprises a nucleotide sequence encoding an amino acid sequence of SEQ ID NO: 13, or an amino acid sequence having at least 70, 75, 80, 85, 90, 95, 96, 97, 98, or 99% identity. In some embodiments, the nucleic acid sequence encoding Sox2 comprises a nucleotide sequence encoding an amino acid sequence of SEQ ID NO: 14, or an amino acid sequence having at least 70, 75, 80, 85, 90, 95, 96, 97, 98, or 99% identity.

In some embodiments, the AAVs comprise a nucleic acid comprising the nucleic acids between the AAV ITRs of SEQ ID NO:10 (i.e., nucleotides 2459-5955), or nucleic acid sequences having at least 70, 75, 80, 85, 90, 95, 96, 97, 98, or 99% identity. In some embodiments, the AAVs comprise a nucleic acid consisting of the nucleic acids between the AAV ITRs of SEQ ID NO:10 (i.e., nucleotides 2459-5955). In some embodiments, the AAVs comprise a nucleic acid comprising the nucleic acids between the AAV ITRs of SEQ ID NO:11 (i.e., nucleotides 2459-6120), or nucleic acid sequences having at least 70, 75, 80, 85, 90, 95, 96, 97, 98, or 99% identity. In some embodiments, the AAVs of the first composition comprise a nucleic acid consisting of the nucleic acids between the AAV ITRs of SEQ ID NO:11 (i.e., nucleotides 2459-6120). In some embodiments, the AAVs comprise a nucleic acid comprising the nucleic acids between the AAV ITRs of SEQ ID NO:10 (i.e., nucleotides 2459-5955), or nucleic acid sequences having at least 70, 75, 80, 85, 90, 95, 96, 97, 98, or 99% identity and except that the CDS of Isl1 is replaced with a nucleic acid sequence encoding Phox2a. In some embodiments, the AAVs comprise a nucleic acid comprising the nucleic acids between the AAV ITRs of SEQ ID NO:10 (i.e., nucleotides 2459-5955), or nucleic acid sequences having at least 70, 75, 80, 85, 90, 95, 96, 97, 98, or 99% identity and except that the CDS of Isl1 is replaced with a nucleic acid sequence encoding Oct4. In some embodiments, the AAVs comprise a nucleic acid comprising the nucleic acids between the AAV ITRs of SEQ ID NO:10 (i.e., nucleotides 2459-5955), or nucleic acid sequences having at least 70, 75, 80, 85, 90, 95, 96, 97, 98, or 99% identity and except that the CDS of Isl1 is replaced with a nucleic acid sequence encoding Sox2.

Programs and algorithms for sequence alignment and comparison of % identity and/or homology between nucleic acid sequences, or polypeptides, are well known in the art, and include BLAST, SIM alignment tool, and so forth.

In some embodiments, the invention is directed to a nucleic acid sequence comprising from about 10 to about 50 consecutive nucleotides from SEQ ID NO: 1.

In some embodiments, the invention is directed to an isolated nucleic acid sequence comprising from about 10 to about 100 consecutive nucleotides from any one of SEQ ID NO: 1 or a sequence complementary to SEQ ID NO: 1. In some embodiments, the invention is directed to an isolated nucleic acid sequence comprising from about 10 to about 200 consecutive nucleotides from any one of SEQ ID NO: 1 or a sequence complementary to SEQ ID NO: 1. In some embodiments, the invention is directed to an isolated nucleic acid sequence comprising from about 10 to about 300 consecutive nucleotides from any one of SEQ ID NO: 1 or a sequence complementary to SEQ ID NO: 1. In some embodiments, the invention is directed to an isolated nucleic acid sequence comprising from about 10 to about 400 consecutive nucleotides from SEQ ID NO: 1 or a sequence complementary to SEQ ID NO: 1. In some embodiments, the invention is directed to an isolated nucleic acid sequence comprising from about 10 to about 500 consecutive nucleotides from any one of SEQ ID NO: 1 or a sequence complementary to SEQ ID NO: 1. In some embodiments, the invention is directed to an isolated nucleic acid sequence comprising from about 10 to about 600 consecutive nucleotides from any one of SEQ ID NO: 1 or a sequence complementary to SEQ ID NO: 1. In some embodiments, the invention is directed to an isolated nucleic acid sequence comprising from about 10 to about 700 consecutive nucleotides from any one of SEQ ID NO: 1 or a sequence complementary to SEQ ID NO: 1. In some embodiments, the invention is directed to an isolated nucleic acid sequence comprising from about 10 to about 800 consecutive nucleotides from any one of SEQ ID NO: 1 or a sequence complementary to SEQ ID NO: 1. In some embodiments, the invention is directed to an isolated nucleic acid sequence comprising from about 10 to about 900 or more consecutive nucleotides from any one of SEQ ID NO: 1 or a sequence complementary to SEQ ID NO: 1.

The sequence identities can be determined by analysis with a sequence comparison algorithm. Nucleic acid sequence identities (homologies) can be evaluated using any of the variety of sequence comparison algorithms and programs known in the art.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.2.2. or FASTA version 3.0t78 algorithms and the default parameters discussed below can be used.

An example of an algorithm that is suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:402-410, 1990, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (www ncbi.nlm.nih.gov/). The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. U.S.A. 89:10915, 1989) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Natl. Acad. Sci. U.S.A. 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, less than about 0.01, and less than about 0.001.

Percent identity in the context of two or more nucleic acids, refers to the percentage of nucleotides that two or more sequences or subsequences contain which are the same. A specified percentage of nucleotides can be referred to such as: 60% identity, 65%, 70%, 75%, 80%, 85%, 90%, 91%, 92%, 93%, 94%, 95%, 96%, 97%, 98%, or 99% or more identity over a specified region, when compared and aligned for maximum correspondence over a comparison window, or designated region as measured using one of the following sequence comparison algorithms or by manual alignment and visual inspection.

In some embodiments, the enhancer sequence drives expression of the polypeptide of interest or oligonucleotide of interest (e.g., a transcription factor that controls gene expression in nascent motor neurons such as Lhx3, Isl1, Phox2a, Oct4, or Sox2) in a cholinergic neuron. In some embodiments, the cholinergic neuron is a motor neuron. In some embodiments, the cholinergic neuron is a basal forebrain cholinergic neuron. In some embodiments, the enhancer sequence drives expression of the polypeptide of interest or oligonucleotide of interest (e.g., a transcription factor that controls gene expression in nascent motor neurons such as Lhx3, Isl1. Phox2a, Oct4, or Sox2) in a cholinergic neuron of an adult subject.

In some embodiments, the enhancer sequence is at least 80%, identical to SEQ ID NO: 1. In some embodiments, the enhancer sequence is at least 85%, identical to SEQ ID NO: 1. In some embodiments, the enhancer sequence is at least 90%, identical to SEQ ID NO: 1. In some embodiments, the enhancer sequence is at least 95%, identical to SEQ ID NO: 1. In some embodiments, the enhancer sequence is at least 96%, identical to SEQ ID NO: 1. In some embodiments, the enhancer sequence is at least 97%, identical to SEQ ID NO: 1. In some embodiments, the enhancer sequence is at least 98%, identical to SEQ ID NO: 1. In some embodiments, the enhancer sequence is at least 99%, identical to SEQ ID NO: 1. In some embodiments, the enhancer sequence comprises SEQ ID NO: 1. In some embodiments, the enhancer sequence consists of SEQ ID NO: 1. In some embodiments, the enhancer sequences and their variants described herein are using in combination with a nucleic acid sequence encoding Isl1 or Lhx3. In some embodiments, the nucleic acid sequence encoding Isl1 comprises nucleotides 3844-4890 of SEQ ID NO: 10, or nucleic acid sequences having at least 70, 75, 80, 85, 90, 95, 96, 97, 98, or 99% identity. In some embodiments, the nucleic acid sequence encoding Isl1 comprises a nucleotide sequence encoding an amino acid sequence as encoded by nucleotides 3844-4890 of SEQ ID NO: 10, or an amino acid sequence having at least 70, 75, 80, 85, 90, 95, 96, 97, 98, or 99% identity. In some embodiments, the nucleic acid sequence encoding Isl1 consists of nucleotides 3844-4890 of SEQ ID NO: 10. In some embodiments, the nucleic acid sequence encoding Lhx3 comprises nucleotides 3844-5052 of SEQ ID NO: 11, or nucleic acid sequences having at least 70, 75, 80, 85, 90, 95, 96, 97, 98, or 99% identity. In some embodiments, the nucleic acid sequence encoding Lhx3 comprises a nucleotide sequence encoding an amino acid sequence as encoded by nucleotides 3844-5052 of SEQ ID NO: 11, or an amino acid sequence having at least 70, 75, 80, 85, 90, 95, 96, 97, 98, or 99% identity. In some embodiments, the nucleic acid sequence encoding Lhx3 consists of nucleotides 3844-5052 of SEQ ID NO: 11. In some embodiments, the nucleic acid sequence encoding Phox2a comprises a nucleotide sequence encoding an amino acid sequence of SEQ ID NO: 12, or an amino acid sequence having at least 70, 75, 80, 85, 90, 95, 96, 97, 98, or 99% identity. In some embodiments, the nucleic acid sequence encoding Oct4 comprises a nucleotide sequence encoding an amino acid sequence of SEQ ID NO: 13, or an amino acid sequence having at least 70, 75, 80, 85, 90, 95, 96, 97, 98, or 99% identity. In some embodiments, the nucleic acid sequence encoding Sox2 comprises a nucleotide sequence encoding an amino acid sequence of SEQ ID NO: 14, or an amino acid sequence having at least 70, 75, 80, 85, 90, 95, 96, 97, 98, or 99% identity.

In some embodiments, the nucleic acid comprises a nucleotide sequence encoding a polypeptide of interest (e.g., a transcription factor that controls gene expression in nascent motor neurons such as Lhx3, Isl1, Phox2a, Oct4, or Sox2), wherein the polypeptide of interest is a prophylactic or therapeutic polypeptide, as described further herein.

In some embodiments, the nucleic acid further comprises a nucleotide sequence encoding a selectable marker gene. In some embodiments, the selectable marker gene is an antibiotic resistance gene. In some embodiments, the antibiotic resistance gene is an ampicillin resistance gene. In some embodiments, the antibiotic resistance gene comprises SEQ ID NO: 2. In some embodiments, the antibiotic resistance gene comprises a nucleotide sequence encoding an amino acid sequence of SEQ ID NO: 3.

In some embodiments, the nucleic acid further comprises a nucleotide sequence encoding a promoter sequence positioned between the enhancer sequence and the nucleotide sequence encoding the polypeptide of interest or an oligonucleotide of interest (e.g., a transcription factor that controls gene expression in nascent motor neurons such as Lhx3, Isl1, Phox2a, Oct4, or Sox2). In some embodiments, the promoter sequence is a mini-promoter sequence. In some embodiments, the promoter sequence comprises SEQ ID NO: 6.

In some embodiments, the nucleic acid further comprises a nucleotide sequence of an intron sequence positioned between the enhancer sequence and the nucleotide sequence encoding the polypeptide of interest or an oligonucleotide of interest (e.g., a transcription factor that controls gene expression in nascent motor neurons such as Lhx3, Isl1, Phox2a, Oct4, or Sox2). In some embodiments, the intron is a chimeric intron. In some embodiments, the intron sequence comprises SEQ ID NO: 7.

In some embodiments, the nucleic acid further comprises a nucleotide sequence of a post-transcriptional regulatory element positioned 3′ to the nucleotide sequence encoding the polypeptide of interest (e.g., a transcription factor that controls gene expression in nascent motor neurons such as Lhx3, Isl1, Phox2a, Oct4, or Sox2). In some embodiments, the post-transcriptional regulatory element is further positioned proximal to a polyA sequence. In some embodiments, the post-transcriptional regulatory element comprises a woodchuck hepatitis virus post-transcriptional regulatory element (WPRE). In some embodiments, the post-transcriptional regulatory element comprises SEQ ID NO: 8.

In some embodiments, the nucleic acid further comprises a nucleotide sequence encoding a polypeptide of interest (e.g., a transcription factor that controls gene expression in nascent motor neurons such as Lhx3, Isl1, Phox2a, Oct4, or Sox2) and further comprising a nucleotide sequence of a polyA sequence positioned 3′ to the nucleotide sequence encoding the polypeptide of interest. In some embodiments, the polyA sequence is an SV40 poly A sequence. In some embodiments, the polyA sequence comprises SEQ ID NO: 9.

### Vectors and Constructs

In other aspects, the invention is directed to expression constructs, for example but not limited to plasmids and vectors which comprise the nucleic acid sequence of SEQ ID NO: 1, complementary sequences thereof, and/or variants thereof. In certain embodiments, the expression constructs further comprise the sequence encoding the oligonucleotide or polypeptide of interest (e.g., a transcription factor that controls gene expression in nascent motor neurons such as Lhx3, Isl1, Phox2a, Oct4, or Sox2). Such expression constructs can be prepared by any suitable method known in the art. Such expression constructs are suitable for viral nucleic acid and/or protein expression and purification. In certain embodiments, the expression constructs comprise a nucleic acid sequence of SEQ ID NO: 10 or 11, complementary sequences thereof, and/or variants or fragments thereof. In some embodiments, the expression constructs comprise a nucleic acid comprising the nucleic acids between the AAV ITRs of SEQ ID NO:10 (i.e., nucleotides 2459-5955). In some embodiments, the expression constructs comprise a nucleic acid consisting of the nucleic acids between the AAV ITRs of SEQ ID NO:10 (i.e., nucleotides 2459-5955). In some embodiments, the expression constructs comprise a nucleic acid comprising the nucleic acids between the AAV ITRs of SEQ ID NO:11 (i.e., nucleotides 2459-6120). In some embodiments, the expression constructs comprise a nucleic acid consisting of the nucleic acids between the AAV ITRs of SEQ ID NO:11 (i.e., nucleotides 2459-6120). In some embodiments, the expression constructs comprise a nucleic acid comprising the nucleic acids between the AAV ITRs of SEQ ID NO:10 (i.e., nucleotides 2459-5955), except that the CDS of Isl1 is replaced with a nucleic acid sequence encoding Phox2a. In some embodiments, the expression constructs comprise a nucleic acid comprising the nucleic acids between the AAV ITRs of SEQ ID NO:10 (i.e., nucleotides 2459-5955), except that the CDS of Isl1 is replaced with a nucleic acid sequence encoding Oct4. In some embodiments, the expression constructs comprise a nucleic acid comprising the nucleic acids between the AAV ITRs of SEQ ID NO:10 (i.e., nucleotides 2459-5955), except that the CDS of Isl1 is replaced with a nucleic acid sequence encoding Sox2.

In certain aspects, the invention provides a nucleic acid vector comprising: an enhancer sequence comprising a nucleotide sequence at least 70% identical to SEQ ID NO: 1; and a nucleotide sequence encoding a polypeptide of interest or an oligonucleotide of interest (e.g., a transcription factor that controls gene expression in nascent motor neurons such as Lhx3, Isl1, Phox2a, Oct4, or Sox2), wherein the nucleotide encoding the polypeptide of interest or oligonucleotide of interest is positioned 3′ to the enhancer sequence. In certain embodiments, the nucleic acid vector comprises the nucleic acid features described above. See section titled “Nucleic Acids”.

In certain embodiments, the vector is a viral vector. In some embodiments, the vector is circular, and in other embodiments, the vector is linearized. Viral vectors include adeno-associated viral (AAV), adenoviral, lentiviral, and retroviral vectors. AAVs can infect terminally differentiated cells, establish nuclear episomes without risking insertional mutagenesis, and convey long-term transgene expression and mild immune responses, making them a preferred choice of delivery to neurons. In certain embodiments, an AAV vector may convey transgene expression for at least one week, at least one month, at least four months, at least six months, at least one year, or longer.

In certain embodiments, the AAV vector contains two inverted terminal repeats (ITRs). In some embodiments, the ITRs are AAV2 ITRs. In some embodiments, the AAV2 ITRs comprise SEQ ID NOs: 4 and 5. In some embodiments, the AAV vector comprises a nucleotide sequence encoding a selectable marker such as an antibiotic resistance gene. Exemplary expression vector is the nucleic acid sequence of SEQ ID NOs: 10 and 11, which is a vector for generation of AAVs comprising the cholinergic enhancer sequence of SEQ ID NO: 1 and an Lhx3 or Isl1 sequence positioned 3′ to the cholinergic enhancer sequence such that the cholinergic enhancer drives expression of Lhx3 or Isl1 in cholinergic neurons. In some embodiments, nucleic acid sequences encoding Phox2a, Oct4, or Sox2 are positioned 3′ to the cholinergic enhancer sequence such that the cholinergic enhancer drives expression of Phox2a, Oct4, or Sox2 in cholinergic neurons. Other nucleotide sequences may replace any origin(s) of replication, mini-promoter, chimeric intron, woodchuck hepatitis virus post-transcriptional regulatory element (WPRE), and/or SV40 polyA sequence with other known sequences that perform the same or similar function to these elements which are known in the art.

### Cells and Animals

In another aspect, described herein are cells comprising the nucleic acid, expression cassette, or viral vector as described herein. In certain embodiments, the viral vector is an AAV vector. In some embodiments, the cells may further comprise an AAV Rep gene and an AAV Cap gene. The AAV Rep and Cap genes may be driven by an AAV promoter such as the p19 and p40 promoters, or they may be driven by a heterologous promoter such as a human cytomegalovirus (CMB) immediate-early enhancer and promoter. In some embodiments, the AAV Rep gene and/or AAV Cap gene are AAV2 genes.

In certain embodiments, the cells further comprise AAV helper genes. AAVs require genes from adenovirus to mediate AAV replication and particle production. Helper genes for AAV include the adenovirus E2A, E4, VA, and E1 genes. These helper genes may be transiently express in the cell as a plasmid, or they may be stably integrated in the cell. A cell line commonly used for production of AAV particles is the HEK293 cell line, which contains the adenovirus gene E1. The remaining helper genes are supplied in the form of a helper plasmid. When the AAV vector comprising the nucleotide sequences described herein positioned between two ITRs is expressed in a cell with AAV Rep/Cap genes and the AAV helper genes, the nucleotide sequence positioned between the ITRs is replicated and packaged in an AAV particle to be delivered to target cells such as cholinergic neurons.

Cells comprising these viral vectors may be cultured in any useful media. Cells can be any permissive cell or tissues, which may be derived from mammals, including, but not limited to, cell lines derived from rodent, murine, human, canine, feline, equine, bovine or porcine cell lines. As used herein, a cell or a tissue can include, but is not limited to individual cells, tissues, organs, insect cells, rodent cells, avian cells, mammalian cells, hybridoma cells, primary cells, continuous cell lines, and/or genetically engineered cells. Cell culture media formulations to suitable for culturing cells are known in the art.

An exogenous nucleic acid, for example a nucleic acid comprising SEQ ID NO: 1 and a nucleic acid sequence encoding a transcription factor that controls gene expression in nascent motor neurons such as Lhx3, Isl1, Phox2a, Oct4, or Sox2 or vector containing SEQ ID NO: 1 and a nucleic acid sequence encoding a transcription factor that controls gene expression in nascent motor neurons such as Lhx3, Isl1, Phox2a, Oct4, or Sox2, can be introduced into a cell via a variety of techniques known in the art, for example, but not limited to lipofection, microinjection, calcium phosphate or calcium chloride precipitation, DEAE-dextrin-mediated transfection, or electroporation.

Cells can be primary and secondary cells, which can be obtained from various tissues and include cell types which can be maintained and propagated in culture.

### Polypeptides of Interest and Oligonucleotides of Interest

In certain aspects, described herein are nucleic acids that encode polypeptides of interest and are positioned 3′ of the enhancer sequence such that the enhancer sequence drives expression of the polypeptides of interest. In certain embodiments, the polypeptide is transcription factors targeting gene induction in nascent motor neurons, such as Lhx3, Isl1, Phox2a. Oct4, or Sox2. In certain embodiments, the polypeptide is Lhx1. In certain embodiments, the polypeptide is Isl1. In some embodiments, the nucleic acid sequence encoding Isl1 comprises nucleotides 3844-4890 of SEQ ID NO: 10. In some embodiments, the nucleic acid sequence encoding Isl1 comprises a nucleotide sequence encoding an amino acid sequence as encoded by nucleotides 3844-4890 of SEQ ID NO: 10. In some embodiments, the nucleic acid sequence encoding Isl1 consists of nucleotides 3844-4890 of SEQ ID NO: 10. In some embodiments, the nucleic acid sequence encoding Lhx3 comprises nucleotides 3844-5052 of SEQ ID NO: 11. In some embodiments, the nucleic acid sequence encoding Lhx3 comprises a nucleotide sequence encoding an amino acid sequence as encoded by nucleotides 3844-5052 of SEQ ID NO: 11. In some embodiments, the nucleic acid sequence encoding Lhx3 consists of nucleotides 3844-5052 of SEQ ID NO: 11. In some embodiments, the nucleic acid sequence encoding Phox2a comprises a nucleotide sequence encoding an amino acid sequence of SEQ ID NO: 12. In some embodiments, the nucleic acid sequence encoding Oct4 comprises a nucleotide sequence encoding an amino acid sequence of SEQ ID NO: 13. In some embodiments, the nucleic acid sequence encoding Sox2 comprises a nucleotide sequence encoding an amino acid sequence of SEQ ID NO: 14.

In certain embodiments, the oligonucleotide of interest is a guide RNA (gRNA) or single guide RNA (sgRNA). The CRISPR/Cas9 gene editing technique promotes a new human gene therapy strategy by correcting a defect gene at pre-chosen sites without altering the endogenous regulation of the target gene. This system consists of two key components: Cas9 protein and a guide RNA, e.g., a single guide RNA (sgRNA), as well as a correction template when needed. sgRNA contains two components: a 17-20 nucleotide sequence termed crispr RNA that is complementary to the target DNA region, and a tracr RNA that serves as the binding scaffold for a Cas nuclease. The sgRNA recognizes the target DNA and guides the Cas9 nuclease to the region for editing.

Methods of Treating and/or Preventing Neurologic Disorders

In another aspect, described herein is a method of treating neurologic disorders in a subject in need thereof comprising administering to a subject a therapeutically effective amount of an AAV particle comprising the nucleotide sequence at least 70% identical to SEQ ID NO: 1 and a nucleic acid sequence encoding a transcription factor that controls gene expression in nascent motor neurons such as Lhx3, Isl1, Phox2a, Oct4, or Sox2 or a pharmaceutical composition comprising said AAV particle. In certain embodiments, the subject is a mammal. In some embodiments, the subject is a mouse or a rat, and preferably the subject is a human. Neurologic disorders that may preferentially benefit from the disclosures herein include neurologic disorders associated with a defect in cholinergic neurons. Such cholinergic neurons may include basal forebrain cholinergic neurons and lower motor neurons. Such neurologic disorders include, but are not limited to, Amyotrophic Lateral Sclerosis (ALS), Alzheimer's disease, Parkinson's disease, Myasthenia gravis, Huntington's chorea, Spinal Muscular Atrophy, Kennedy Disease, Progressive Muscular Atrophy, and Monomelic Amyotrophy. In some embodiments, the neurologic disorder is Alzheimer's disease. In some embodiments, the neurologic disorder is ALS.

In certain aspects, described herein is a method for rejuvenating spinal motor neurons or increasing motor neuron resistance to ALS pathogens in a subject with ALS. In some embodiments, the method uses an enhancer specific to a subset of motor neurons, such as lower motor neurons or upper motor neurons, and is packaged in AAVs. In some embodiments, a subject is administered the AAVs containing the enhancer to induce gene expression or knockdown in spinal motor neurons at different developmental stages. In certain embodiments, the enhancer is a nucleotide sequence at least 70% identical to SEQ ID NO: 1. In some embodiments, the enhancer sequence drives a nucleotide sequence encoding a polypeptide of interest or an oligonucleotide of interest (e.g., a transcription factor that controls gene expression in nascent motor neurons such as Lhx3, Isl1, Phox2a, Oct4, or Sox2), wherein the nucleotide sequence encoding the polypeptide of interest or oligonucleotide of interest is positioned 3′ to the enhancer sequence. In certain embodiments, the polypeptide of interest or oligonucleotide of interest are the polypeptides and oligonucleotides described above herein.

Cholinergic neurons are neurons that utilize the neurotransmitter acetylcholine, synthesized through activity of the enzyme choline acetyltransferase (ChAT) to send messages. Cholinergic neurons are distributed throughout the central nervous system, including but not limited to the spinal cord, striatum, hindbrain, and basal forebrain. One subset of cholinergic neurons are spinal motor neurons or lower motor neurons. These neurons are involved in sensory, autonomic and motor control in the spinal cord. Degeneration of these neurons is a characteristic of ALS. Another subset of cholinergic neurons are basal forebrain cholinergic neurons. These neurons are critical for a range of cognitive functions, and loss of these neurons is characteristic of Alzheimer's Disease.

In certain embodiments, the AAV particles are administered by injection, which may include, but is not limited to, intravenous, intracerebroventricular, intrathecal, intraparenchymal, intramuscular, or intraperitoneal injection.

Therapeutically effective amount refers to an amount that is effective for preventing, ameliorating, treating or delaying the onset of a disease or condition. The pharmaceutical compositions (e.g. comprising an AAV particle described herein) of the inventions can be administered to any animal that can experience the beneficial effects of the agents of the invention. Such animals include humans and non-humans.

Routes of administration and dosages of effective amounts of the pharmaceutical compositions (e.g. comprising an AAV particle described herein) are also disclosed. The agents of the present invention can be administered in combination with other pharmaceutical agents in a variety of protocols for effective treatment of disease.

Pharmaceutical compositions (e.g. comprising an AAV particle described herein) are administered to a subject in a manner known in the art. The dosage administered will be dependent upon the age, health, and weight of the recipient, kind of concurrent treatment, if any, frequency of treatment, and the nature of the effect desired. One may administer the pharmaceutical compositions in a local rather than systemic manner, for example, via injection of directly into the desired target site, often in a depot or sustained release formulation.

One of ordinary skill in the art will appreciate that a method of administering pharmaceutically effective amounts of the pharmaceutical compositions (e.g. comprising an AAV particle described herein) to a patient in need thereof, can be determined empirically, or by standards currently recognized in the medical arts. The AAV particles can be administered to a patient as pharmaceutical compositions in combination with one or more pharmaceutically acceptable excipients. It will be understood that, when administered to a human patient, the total daily usage of the agents of the pharmaceutical compositions (e.g. comprising an AAV particle described herein) will be decided within the scope of sound medical judgment by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, gender and diet of the patient; the time of administration, route of administration, and rate of excretion of the agent; the duration of the treatment; drugs used in combination or coincidental with the specific agent; and like factors well known in the medical arts. It is well within the skill of the art to start doses of the agents at levels lower than those required to achieve the desired therapeutic effect and to gradually increase the dosages until the desired effect is achieved.

### Enhancer to Control Gene Expression in a Subset of Cholinergic Neurons

A commonly studied promoter to control gene expression in cholinergic neurons is the HB9 promoter described in U.S. Pat. No. 7,632,679 and S. Arber et al., Requirement for the Homeobox Gene Hb9 in the Consolidation of Motor Neuron Identity, Neuron. 1999 August; 23(4):659-74, each of which are herein incorporated by reference in their entirety. Key disadvantages of the HB9 promoter include its large (9.5 kb) size, hindering its compatibility with viral packaging, and the fact that it is only active during early postnatal stages. Various groups have attempted to identify shorter segments of the HB9 promoter, but these enhancers control expression only in nascent embryonic motor neurons. There is currently an effort in the field to develop a new enhancer that can be used to control gene expression specifically in a subset of cholinergic neurons. These cholinergic neurons include motor neurons affected in ALS as well as basal forebrain cholinergic neurons affected in AD and other diseases. The cholinergic enhancer disclosed in SEQ ID NO: 1 was identified by mapping accessible chromatin regions around the CHAT gene. Importantly, this enhancer is much smaller (1 kb) than the HB9 promoter. This makes the cholinergic enhancer fully compatible with viral packaging. Additionally, this regulatory element can be used to drive expression in cholinergic neurons at all ages from early postnatal to adulthood. Exemplary validation of the cholinergic enhancer disclosed in SEQ ID NO: 1 is provided in application PCT/US23/69780, to which the present application claims priority and the contents of which are hereby incorporated by reference in their entirety, including Examples 1-5.

### Non-Limiting Embodiments of the Subject Matter

In certain aspects, the present disclosure provides a method for treating Amyotrophic Lateral Sclerosis (ALS) in a subject in need thereof, the method comprising: administering to the subject a composition comprising adeno-associated viruses (AAVs), wherein the AAVs comprise a nucleic acid sequence comprising an enhancer sequence and encoding one or more transcription factors that control gene expression in nascent motor neurons, wherein the enhancer is capable of driving a motor-neuron specific expression of the one or more transcription factors and wherein the one or more transcription factors are expressed in motor neurons of the subject.

In some embodiments, the motor neurons are rejuvenated, and/or their resistance to ALS pathogens in the subject with ALS is increased.

In some embodiments, the enhancer sequence comprises SEQ ID NO: 1. In some embodiments, the enhancer sequence consists of SEQ ID NO: 1. In some embodiments, the enhancer is ChatE.

In some embodiments, the one or more transcription factors is Lhx3. In some embodiments, the one or more transcription factors is Isl1. In some embodiments, the one or more transcription factors are Isl1 and Lhx3. In some embodiments, the nucleic acid sequence encoding Isl1 comprises nucleotides 3844-4890 of SEQ ID NO: 10. In some embodiments, the nucleic acid sequence encoding Isl1 comprises a nucleotide sequence encoding an amino acid sequence as encoded by nucleotides 3844-4890 of SEQ ID NO: 10. In some embodiments, the nucleic acid sequence encoding Isl1 consists of nucleotides 3844-4890 of SEQ ID NO: 10. In some embodiments, the nucleic acid sequence encoding Lhx3 comprises nucleotides 3844-5052 of SEQ ID NO: 11. In some embodiments, the nucleic acid sequence encoding Lhx3 comprises a nucleotide sequence encoding an amino acid sequence as encoded by nucleotides 3844-5052 of SEQ ID NO: 11. In some embodiments, the nucleic acid sequence encoding Lhx3 consists of nucleotides 3844-5052 of SEQ ID NO: 11. In some embodiments, the one or more transcription factors is Phox2a. In some embodiments, the one or more transcription factors is Sox2. In some embodiments, the one or more transcription factors are Phox2a and Isl1. In some embodiments, the one or more transcription factors is Oct4. In some embodiments, the one or more transcription factors are Oct4 and Sox2.

In some embodiments, the AAVs comprise a nucleic acid comprising the nucleic acids between the AAV ITRs of SEQ ID NO:10 (i.e., nucleotides 2459-5955). In some embodiments, the AAVs comprise a nucleic acid consisting of the nucleic acids between the AAV ITRs of SEQ ID NO:10 (i.e., nucleotides 2459-5955). In some embodiments, the AAVs comprise a nucleic acid comprising the nucleic acids between the AAV ITRs of SEQ ID NO:11 (i.e., nucleotides 2459-6120). In some embodiments, the AAVs comprise a nucleic acid consisting of the nucleic acids between the AAV ITRs of SEQ ID NO:11 (i.e., nucleotides 2459-6120).

In some embodiments, the re-expression of one or more transcription factors reactivates their embryonic targets. In some embodiments, the embryonic target MNX1.

In some embodiments, the motor neurons are spinal motor neurons.

In some embodiments, the AAVs are capable of penetrating the blood-brain barrier.

In some embodiments, administration of the AAVs attenuates disease-related proteinopathies in the motor neurons. In some embodiments, administration of the AAVs reduces the formation of p62+ aggregates in the motor neurons. In some embodiments, administration of the AAVs reduces the incidence of SQSTM1-positive round bodies. In some embodiments, administration of the AAVs reduces the formation of SOD1+ aggregates in the motor neurons. In some embodiments, administration of the AAVs results in the reduction of neuroinflammation in the vicinity of motor neurons. In some embodiments, administration of the AAVs results in the reduction of Iba1+ microglia activation in the vicinity of the motor neurons.

In some embodiments, administration of the AAVs ameliorates clinical phenotypes of ALS. In some embodiments, administration of the AAVs delays symptom onset of ALS.

In certain aspects, the present disclosure provides a method for treating Amyotrophic Lateral Sclerosis (ALS) in a subject in need thereof, the method comprising: administering to the subject a first composition comprising adeno-associated viruses (AAVs) and a second composition comprising AAVs, wherein the AAVs of the first composition comprise a nucleic acid sequence comprising an enhancer sequence and encoding a first transcription factor that controls gene expression in nascent motor neurons and wherein the enhancer is capable of driving a motor-neuron specific expression of the first transcription factor, wherein the AAVs of the second composition comprise a nucleic acid sequence comprising an enhancer sequence and encoding a second transcription factor that controls gene expression in nascent motor neurons wherein the enhancer is capable of driving a motor-neuron specific expression of the second transcription factor, and wherein the first and second transcription factors are expressed in motor neurons of the subject.

In some embodiments, the motor neurons are rejuvenated, and/or their resistance to ALS pathogens in the subject with ALS is increased.

In some embodiments, the enhancer sequence comprises SEQ ID NO: 1. In some embodiments, the enhancer sequence consists of SEQ ID NO: 1. In some embodiments, the enhancer is ChatE.

In some embodiments, the first transcription factor is Lhx3. In some embodiments, the second transcription factor is Isl1. In some embodiments, the first transcription factor is Isl1 and the second transcription factor is Lhx3. In some embodiments, the nucleic acid sequence encoding Isl1 comprises nucleotides 3844-4890 of SEQ ID NO: 10. In some embodiments, the nucleic acid sequence encoding Isl1 comprises a nucleotide sequence encoding an amino acid sequence as encoded by nucleotides 3844-4890 of SEQ ID NO: 10. In some embodiments, the nucleic acid sequence encoding Isl1 consists of nucleotides 3844-4890 of SEQ ID NO: 10. In some embodiments, the nucleic acid sequence encoding Lhx3 comprises nucleotides 3844-5052 of SEQ ID NO: 11. In some embodiments, the nucleic acid sequence encoding Lhx3 comprises a nucleotide sequence encoding an amino acid sequence as encoded by nucleotides 3844-5052 of SEQ ID NO: 11. In some embodiments, the nucleic acid sequence encoding Lhx3 consists of nucleotides 3844-5052 of SEQ ID NO: 11. In some embodiments, the one or more transcription factors is Phox2a. In some embodiments, the one or more transcription factors is Sox2. In some embodiments, the one or more transcription factors are Phox2a and Isl1. In some embodiments, the one or more transcription factors is Oct4. In some embodiments, the one or more transcription factors are Oct4 and Sox2.

In some embodiments, the AAVs of the second composition comprise a nucleic acid comprising the nucleic acids between the AAV ITRs of SEQ ID NO:10 (i.e., nucleotides 2459-5955). In some embodiments, the AAVs of the second composition comprise a nucleic acid consisting of the nucleic acids between the AAV ITRs of SEQ ID NO:10 (i.e., nucleotides 2459-5955). In some embodiments, the AAVs of the first composition comprise a nucleic acid comprising the nucleic acids between the AAV ITRs of SEQ ID NO:11 (i.e., nucleotides 2459-6120). In some embodiments, the AAVs of the first composition comprise a nucleic acid consisting of the nucleic acids between the AAV ITRs of SEQ ID NO:11 (i.e., nucleotides 2459-6120).

In some embodiments, the re-expression of one or more transcription factors reactivates their embryonic targets. In some embodiments, the embryonic target MNX1.

In some embodiments, the motor neurons are spinal motor neurons.

In some embodiments, the AAVs are capable of penetrating the blood-brain barrier.

In some embodiments, administration of the AAVs attenuates disease-related proteinopathies in the motor neurons. In some embodiments, administration of the AAVs reduces the formation of p62+ aggregates in the motor neurons. In some embodiments, administration of the AAVs reduces the incidence of SQSTM1-positive round bodies. In some embodiments, administration of the AAVs reduces the formation of SOD1+ aggregates in the motor neurons. In some embodiments, administration of the AAVs results in the reduction of neuroinflammation in the vicinity of motor neurons. In some embodiments, administration of the AAVs results in the reduction of Iba1+ microglia activation in the vicinity of the motor neurons.

In some embodiments, administration of the AAVs ameliorates clinical phenotypes of ALS. In some embodiments, administration of the AAVs delays symptom onset of ALS.

In certain aspects, the present disclosure provides a composition for treating ALS in a subject in need thereof, the composition comprising AAVs, wherein the AAVs comprise a nucleic acid sequence comprising an enhancer sequence and encoding one or more transcription factors that control gene expression in nascent motor neurons, wherein the enhancer is capable of driving a motor-neuron specific expression of the one or more transcription factors and wherein the one or more transcription factors are expressed in motor neurons of the subject.

In some embodiments, the motor neurons are rejuvenated, and/or their resistance to ALS pathogens in the subject with ALS is increased.

In some embodiments, the enhancer sequence comprises SEQ ID NO: 1. In some embodiments, the enhancer sequence consists of SEQ ID NO: 1. In some embodiments, the enhancer is ChatE.

In some embodiments, the one or more transcription factors is Lhx3. In some embodiments, the one or more transcription factors is Isl1. In some embodiments, the one or more transcription factors are Isl1 and Lhx3. In some embodiments, the nucleic acid sequence encoding Isl1 comprises nucleotides 3844-4890 of SEQ ID NO: 10. In some embodiments, the nucleic acid sequence encoding Isl1 comprises a nucleotide sequence encoding an amino acid sequence as encoded by nucleotides 3844-4890 of SEQ ID NO: 10. In some embodiments, the nucleic acid sequence encoding Isl1 consists of nucleotides 3844-4890 of SEQ ID NO: 10. In some embodiments, the nucleic acid sequence encoding Lhx3 comprises nucleotides 3844-5052 of SEQ ID NO: 11. In some embodiments, the nucleic acid sequence encoding Lhx3 comprises a nucleotide sequence encoding an amino acid sequence as encoded by nucleotides 3844-5052 of SEQ ID NO: 11. In some embodiments, the nucleic acid sequence encoding Lhx3 consists of nucleotides 3844-5052 of SEQ ID NO: 11. In some embodiments, the one or more transcription factors is Phox2a. In some embodiments, the one or more transcription factors is Sox2. In some embodiments, the one or more transcription factors are Phox2a and Isl1. In some embodiments, the one or more transcription factors is Oct4. In some embodiments, the one or more transcription factors are Oct4 and Sox2.

In some embodiments, the AAVs comprise a nucleic acid comprising the nucleic acids between the AAV ITRs of SEQ ID NO:10 (i.e., nucleotides 2459-5955). In some embodiments, the AAVs comprise a nucleic acid consisting of the nucleic acids between the AAV ITRs of SEQ ID NO:10 (i.e., nucleotides 2459-5955). In some embodiments, the AAVs comprise a nucleic acid comprising the nucleic acids between the AAV ITRs of SEQ ID NO:11 (i.e., nucleotides 2459-6120). In some embodiments, the AAVs comprise a nucleic acid consisting of the nucleic acids between the AAV ITRs of SEQ ID NO:11 (i.e., nucleotides 2459-6120).

In some embodiments, the motor neurons are spinal motor neurons.

In some embodiments, the AAVs are capable of penetrating the blood-brain barrier.

In certain aspects, the present disclosure provides a composition for treating ALS in a subject in need thereof, the composition comprising: a first set of AAVs and a second set of AAVs, wherein the first set of AAVs comprise a nucleic acid sequence comprising an enhancer sequence and encoding a first transcription factor that controls gene expression in nascent motor neurons and wherein the enhancer is capable of driving a motor-neuron specific expression of the first transcription factor, wherein the second set of AAVs comprise a nucleic acid sequence comprising an enhancer sequence and encoding a second transcription factor that controls gene expression in nascent motor neurons wherein the enhancer is capable of driving a motor-neuron specific expression of the second transcription factor, and wherein the first and second transcription factors are expressed in motor neurons of the subject.

In some embodiments, the motor neurons are rejuvenated, and/or their resistance to ALS pathogens in the subject with ALS is increased.

In some embodiments, the enhancer sequence comprises SEQ ID NO: 1. In some embodiments, the enhancer sequence consists of SEQ ID NO: 1. In some embodiments, the enhancer is ChatE.

In some embodiments, the first transcription factor is Lhx3. In some embodiments, the second transcription factor is Isl1. In some embodiments, the first transcription factor is Isl1 and the second transcription factor is Lhx3. In some embodiments, the nucleic acid sequence encoding Isl1 comprises nucleotides 3844-4890 of SEQ ID NO: 10. In some embodiments, the nucleic acid sequence encoding Isl1 comprises a nucleotide sequence encoding an amino acid sequence as encoded by nucleotides 3844-4890 of SEQ ID NO: 10. In some embodiments, the nucleic acid sequence encoding Isl1 consists of nucleotides 3844-4890 of SEQ ID NO: 10. In some embodiments, the nucleic acid sequence encoding Lhx3 comprises nucleotides 3844-5052 of SEQ ID NO: 11. In some embodiments, the nucleic acid sequence encoding Lhx3 comprises a nucleotide sequence encoding an amino acid sequence as encoded by nucleotides 3844-5052 of SEQ ID NO: 11. In some embodiments, the nucleic acid sequence encoding Lhx3 consists of nucleotides 3844-5052 of SEQ ID NO: 11. In some embodiments, the one or more transcription factors is Phox2a. In some embodiments, the one or more transcription factors is Sox2. In some embodiments, the one or more transcription factors are Phox2a and Isl1. In some embodiments, the one or more transcription factors is Oct4. In some embodiments, the one or more transcription factors are Oct4 and Sox2.

In some embodiments, the set of AAVs comprise a nucleic acid comprising the nucleic acids between the AAV ITRs of SEQ ID NO:10 (i.e., nucleotides 2459-5955). In some embodiments, the set of AAVs comprise a nucleic acid consisting of the nucleic acids between the AAV ITRs of SEQ ID NO:10 (i.e., nucleotides 2459-5955). In some embodiments, the set of AAVs comprise a nucleic acid comprising the nucleic acids between the AAV ITRs of SEQ ID NO:11 (i.e., nucleotides 2459-6120). In some embodiments, the set of AAVs comprise a nucleic acid consisting of the nucleic acids between the AAV ITRs of SEQ ID NO:11 (i.e., nucleotides 2459-6120).

In some embodiments, the re-expression of one or more transcription factors reactivates their embryonic targets. In some embodiments, the embryonic target is MNX1.

In some embodiments, the motor neurons are spinal motor neurons.

In some embodiments, the AAVs are capable of penetrating the blood-brain barrier.

In certain aspects, the present disclosure provides a vector for treating ALS in a subject in need thereof, the vector comprising a nucleic acid sequence comprising an enhancer sequence and encoding one or more transcription factors that control gene expression in nascent motor neurons, wherein the enhancer is capable of driving a motor-neuron specific expression of the one or more transcription factors and wherein the one or more transcription factors are expressed in motor neurons of the subject.

In some embodiments, the motor neurons are rejuvenated, and/or their resistance to ALS pathogens in the subject with ALS is increased.

In some embodiments, the enhancer sequence comprises SEQ ID NO: 1. In some embodiments, the enhancer sequence consists of SEQ ID NO: 1. In some embodiments, the enhancer is ChatE.

In some embodiments, the one or more transcription factors is Lhx3. In some embodiments, the one or more transcription factors is Isl1. In some embodiments, the one or more transcription factors are Isl1 and Lhx3. In some embodiments, the nucleic acid sequence encoding Isl1 comprises nucleotides 3844-4890 of SEQ ID NO: 10. In some embodiments, the nucleic acid sequence encoding Isl1 comprises a nucleotide sequence encoding an amino acid sequence as encoded by nucleotides 3844-4890 of SEQ ID NO: 10. In some embodiments, the nucleic acid sequence encoding Isl1 consists of nucleotides 3844-4890 of SEQ ID NO: 10. In some embodiments, the nucleic acid sequence encoding Lhx3 comprises nucleotides 3844-5052 of SEQ ID NO: 11. In some embodiments, the nucleic acid sequence encoding Lhx3 comprises a nucleotide sequence encoding an amino acid sequence as encoded by nucleotides 3844-5052 of SEQ ID NO: 11. In some embodiments, the nucleic acid sequence encoding Lhx3 consists of nucleotides 3844-5052 of SEQ ID NO: 11. In some embodiments, the one or more transcription factors is Phox2a. In some embodiments, the one or more transcription factors is Sox2. In some embodiments, the one or more transcription factors are Phox2a and Isl1. In some embodiments, the one or more transcription factors is Oct4. In some embodiments, the one or more transcription factors are Oct4 and Sox2.

In some embodiments, the vector comprises a nucleic acid comprising the nucleic acids between the AAV ITRs of SEQ ID NO:10 (i.e., nucleotides 2459-5955). In some embodiments, the vector comprises a nucleic acid consisting of the nucleic acids between the AAV ITRs of SEQ ID NO:10 (i.e., nucleotides 2459-5955). In some embodiments, the vector comprises a nucleic acid comprising the nucleic acids between the AAV ITRs of SEQ ID NO:11 (i.e., nucleotides 2459-6120). In some embodiments, the vector comprises a nucleic acid consisting of the nucleic acids between the AAV ITRs of SEQ ID NO:11 (i.e., nucleotides 2459-6120). In some embodiments, the vector comprises SEQ ID NO: 10. In some embodiments, the vector consists of SEQ ID NO: 10. In some embodiments, the vector comprises SEQ ID NO: 11. In some embodiments, the vector consists of SEQ ID NO: 11.

In some embodiments, the motor neurons are spinal motor neurons.

In some embodiments, the AAVs are capable of penetrating the blood-brain barrier.

SEQUENCES

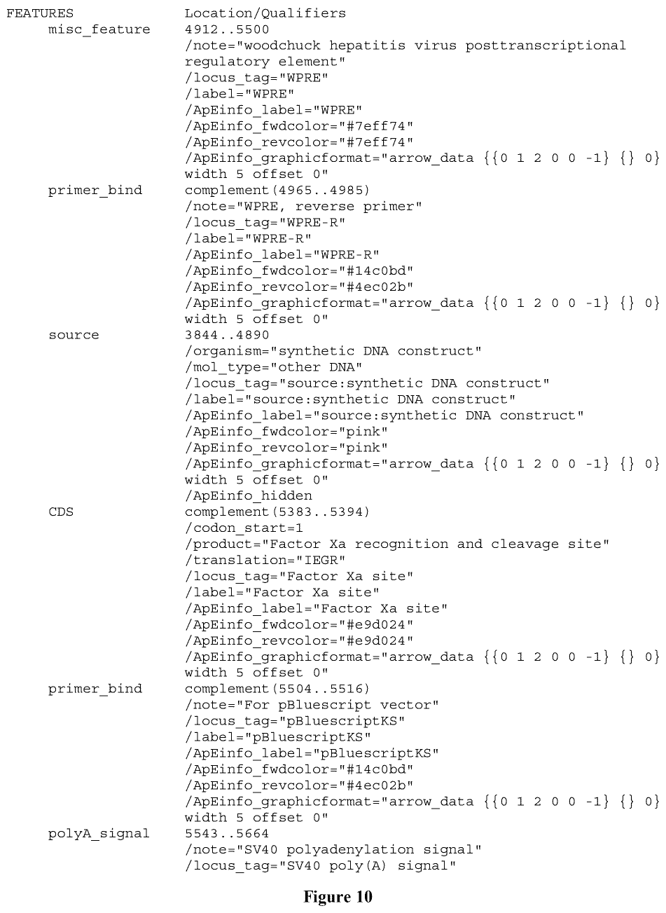

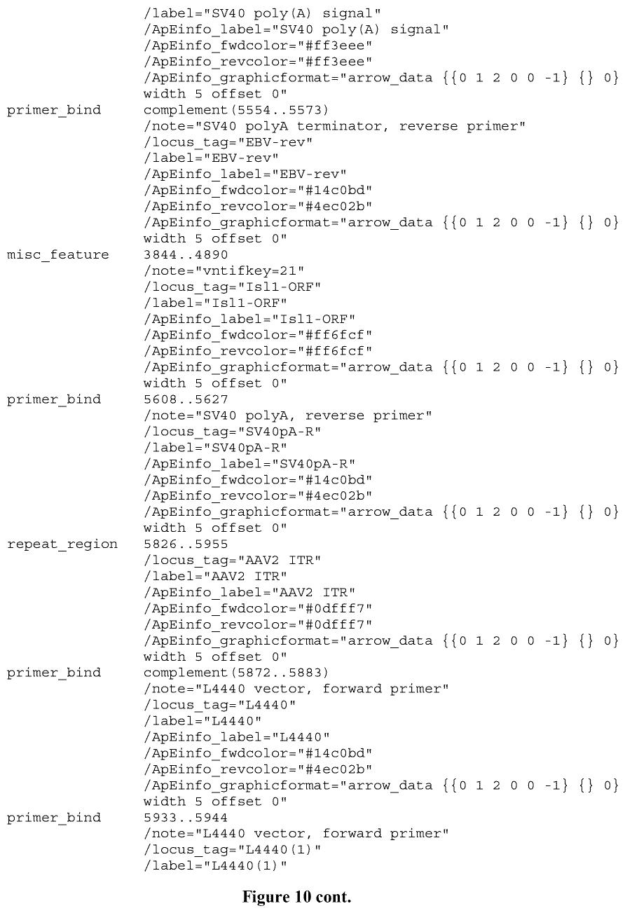

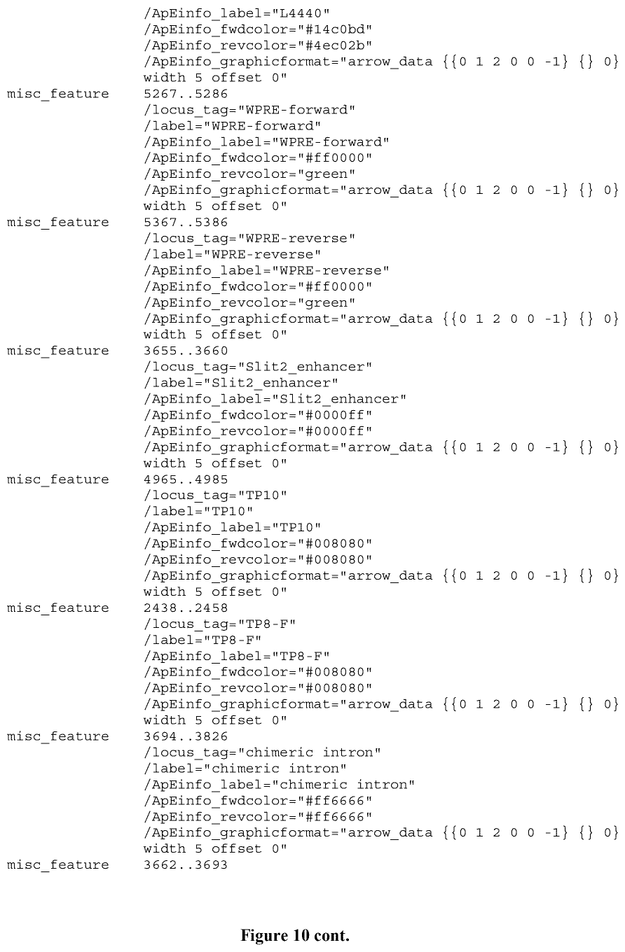

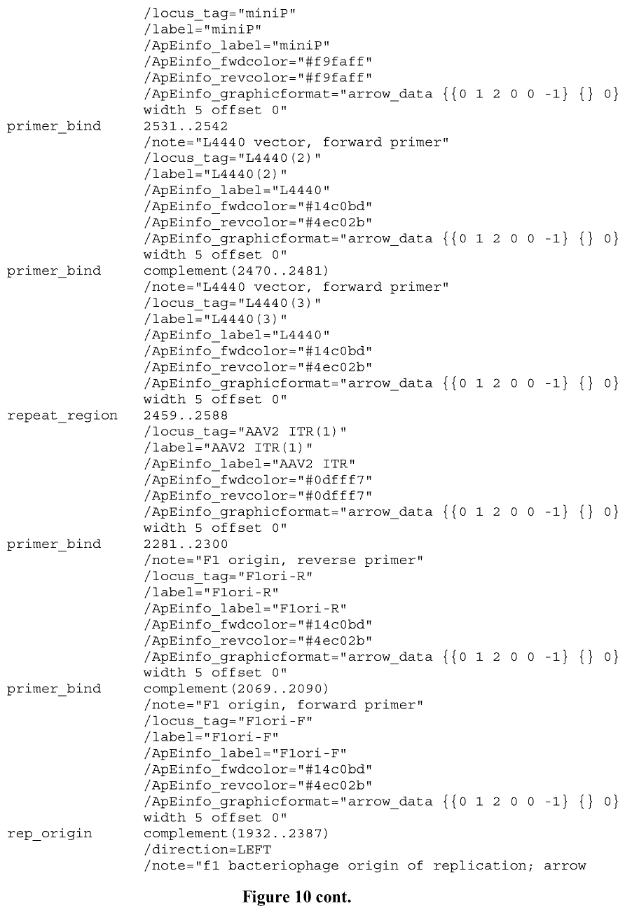

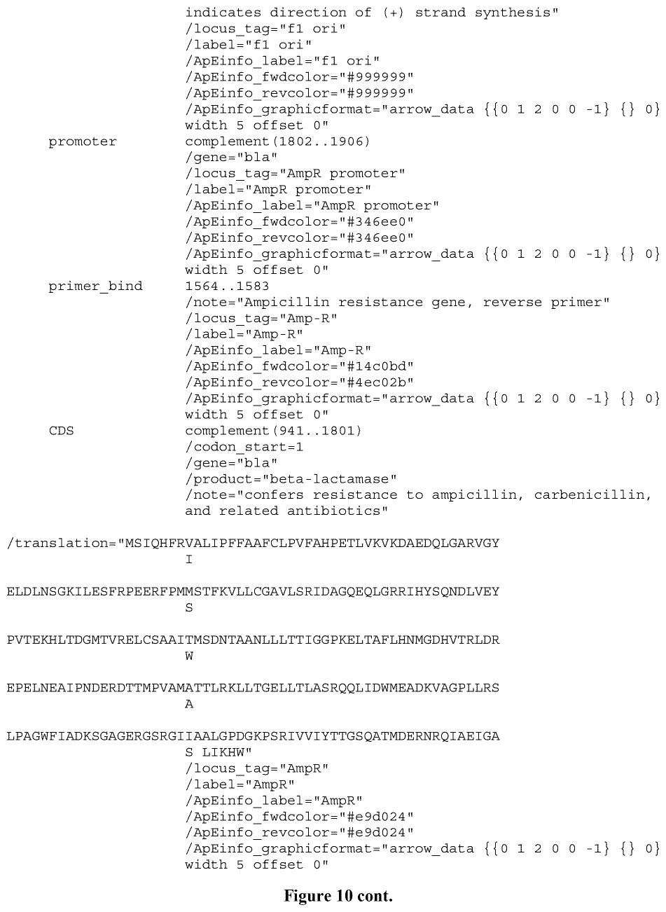

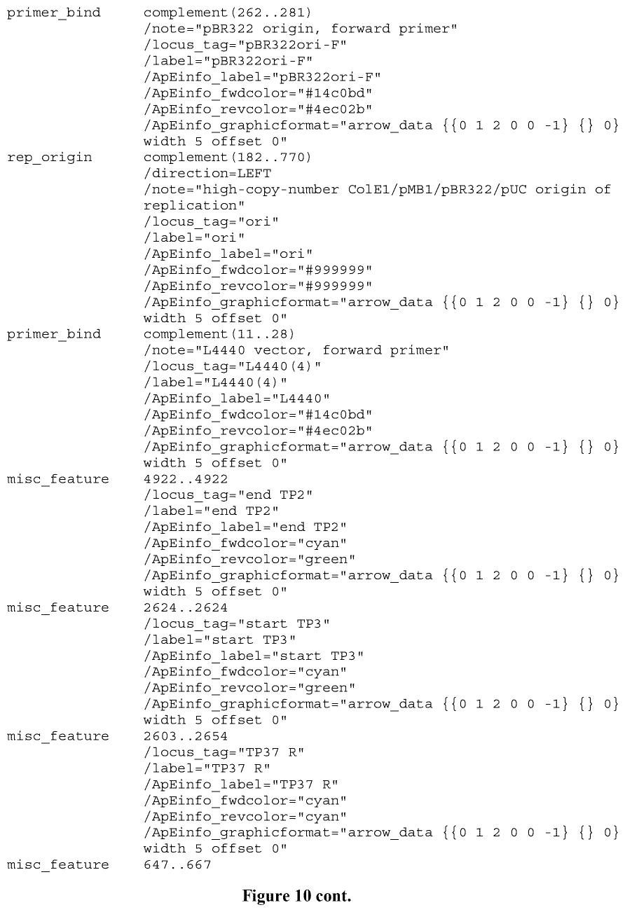

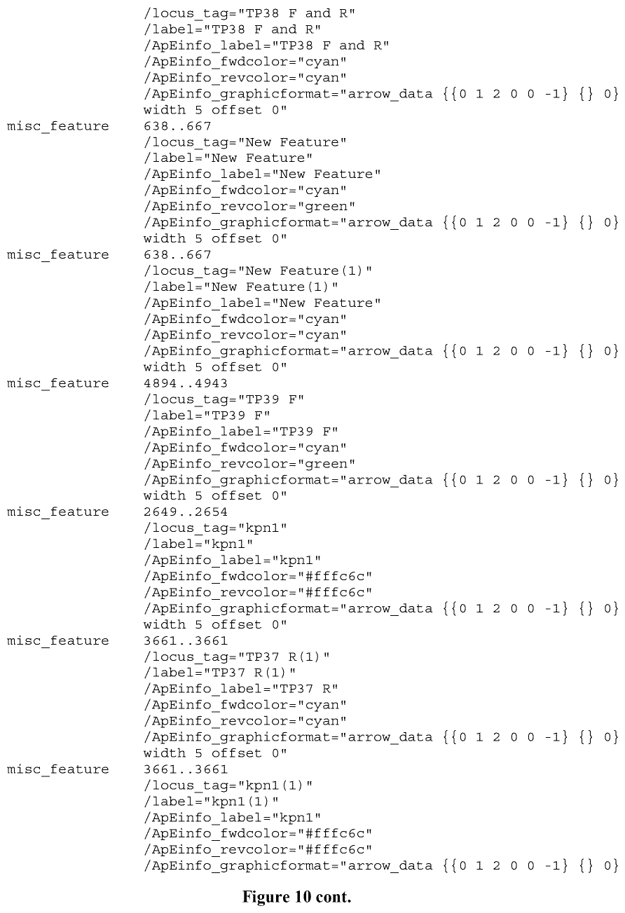

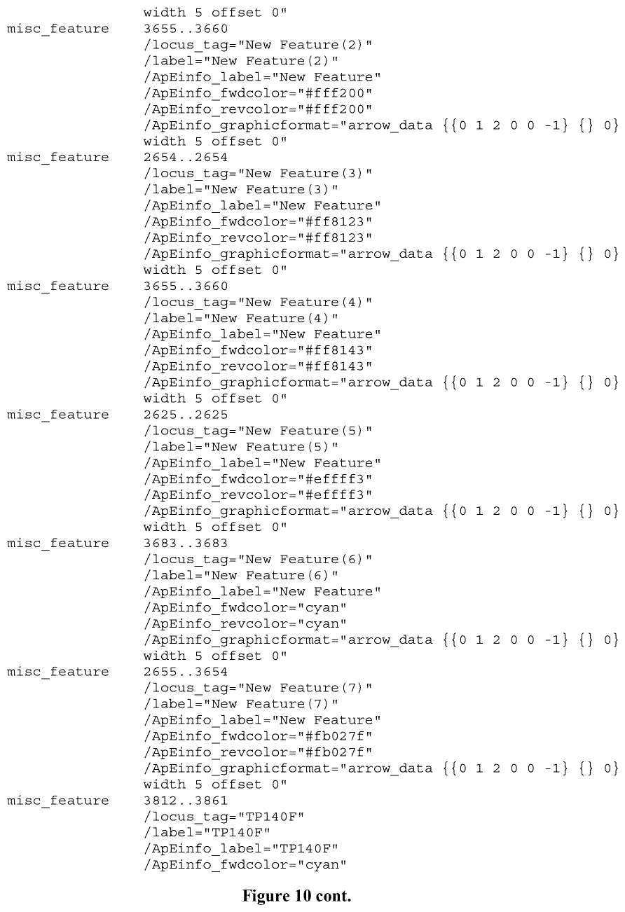

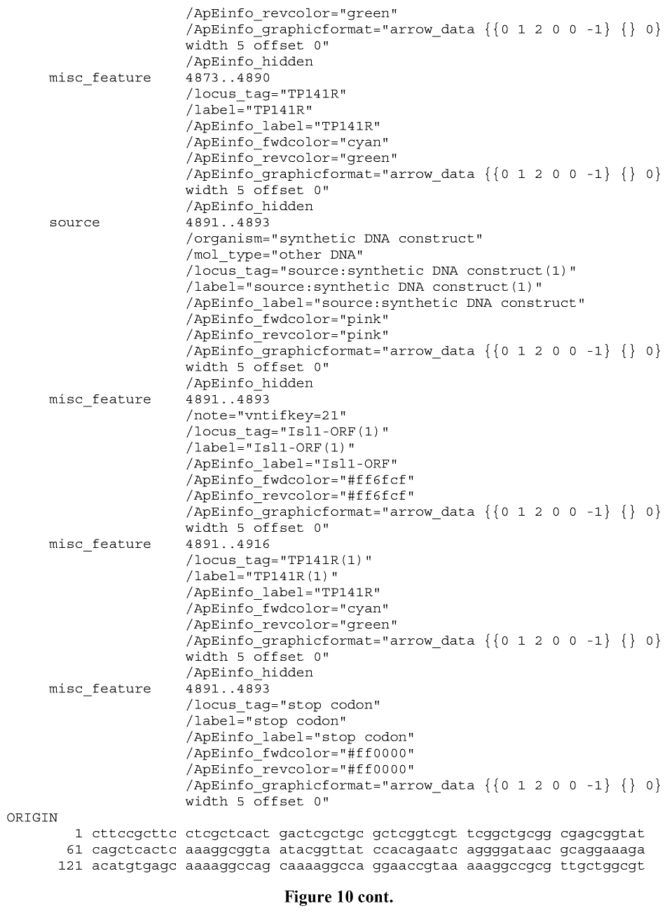

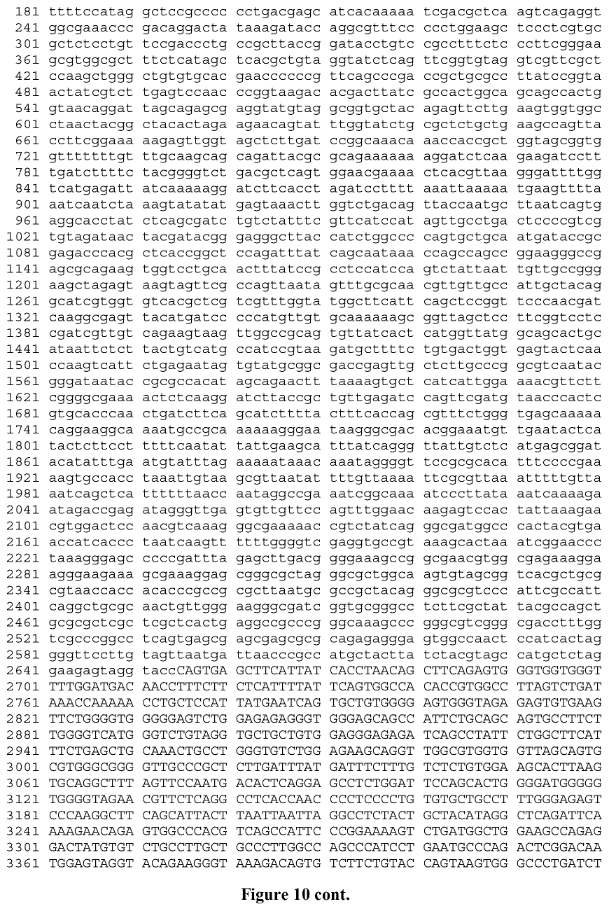

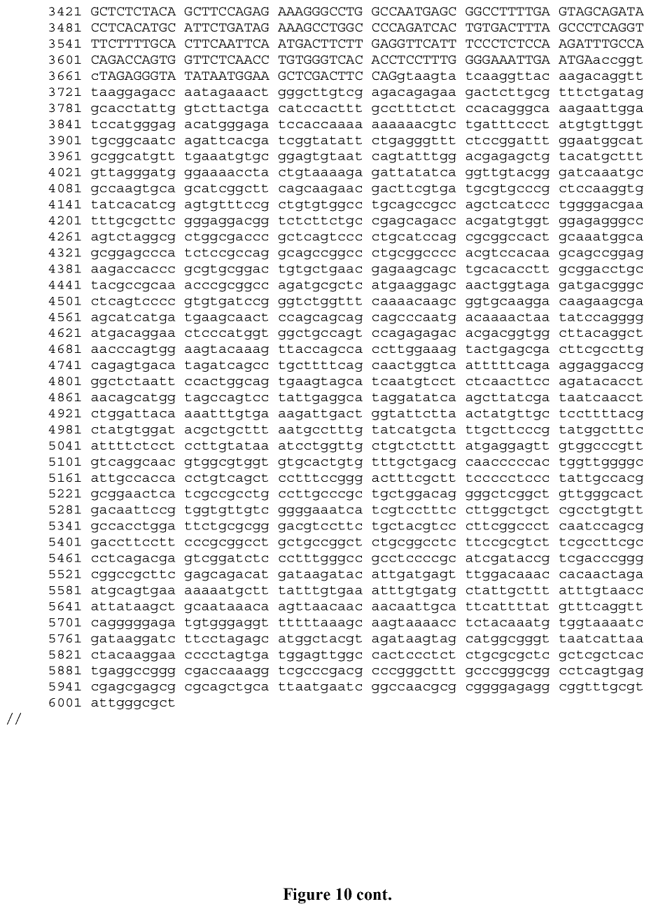

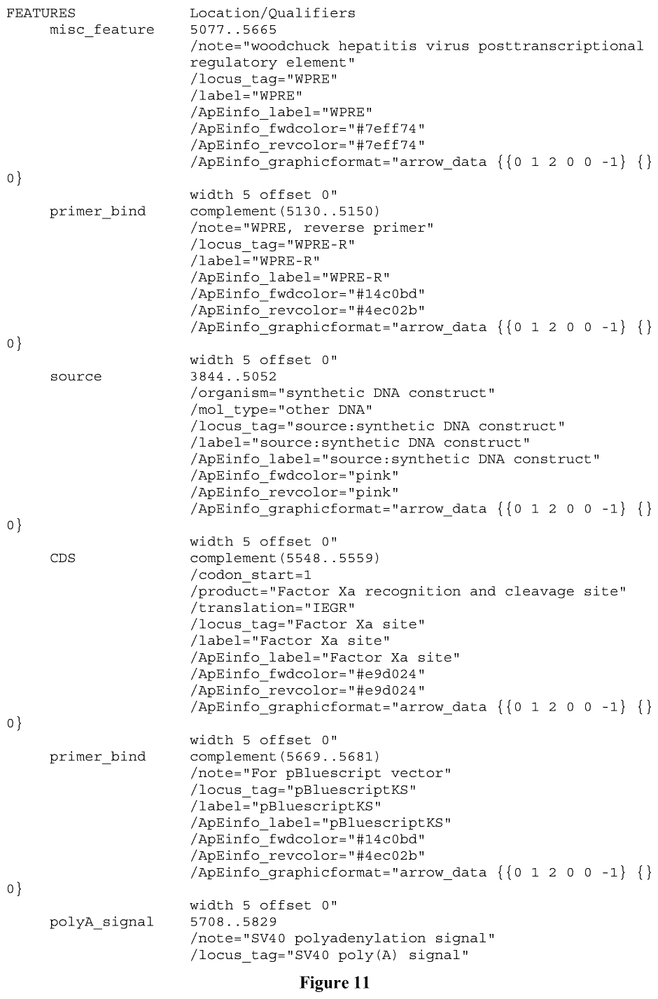

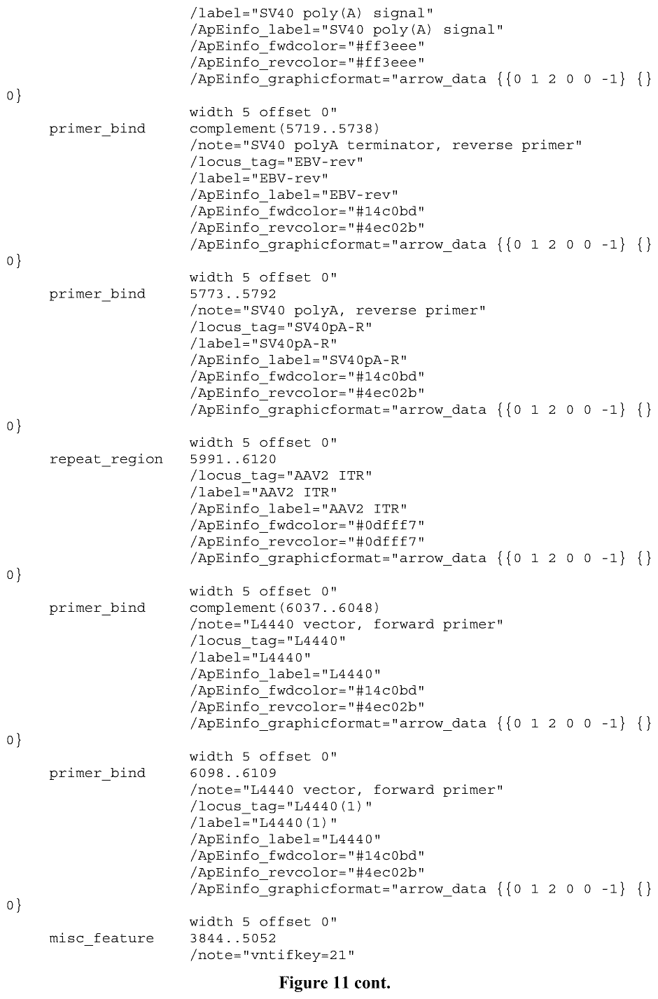

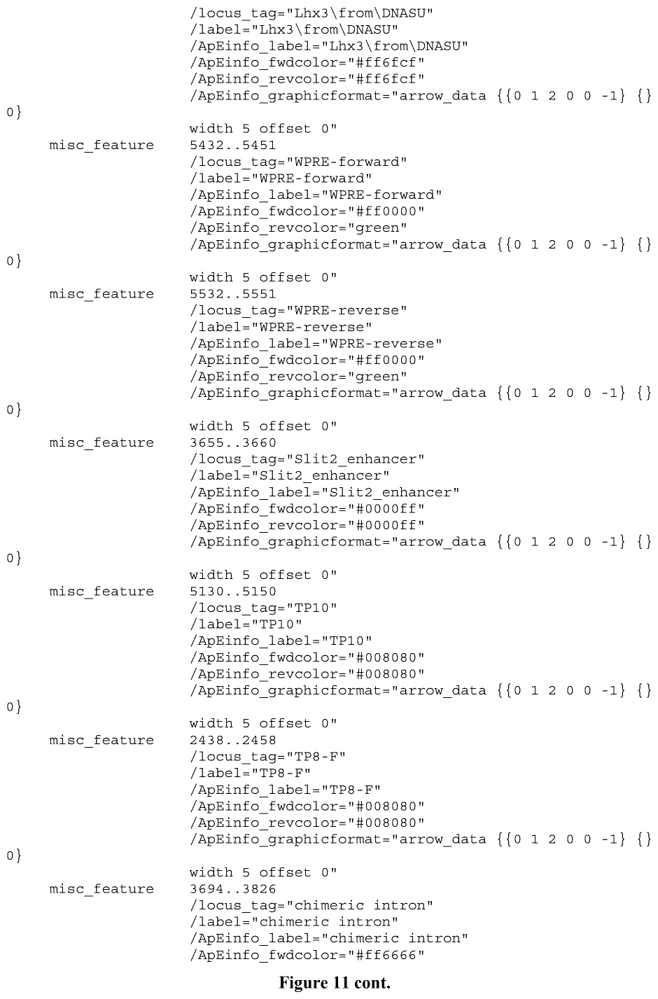

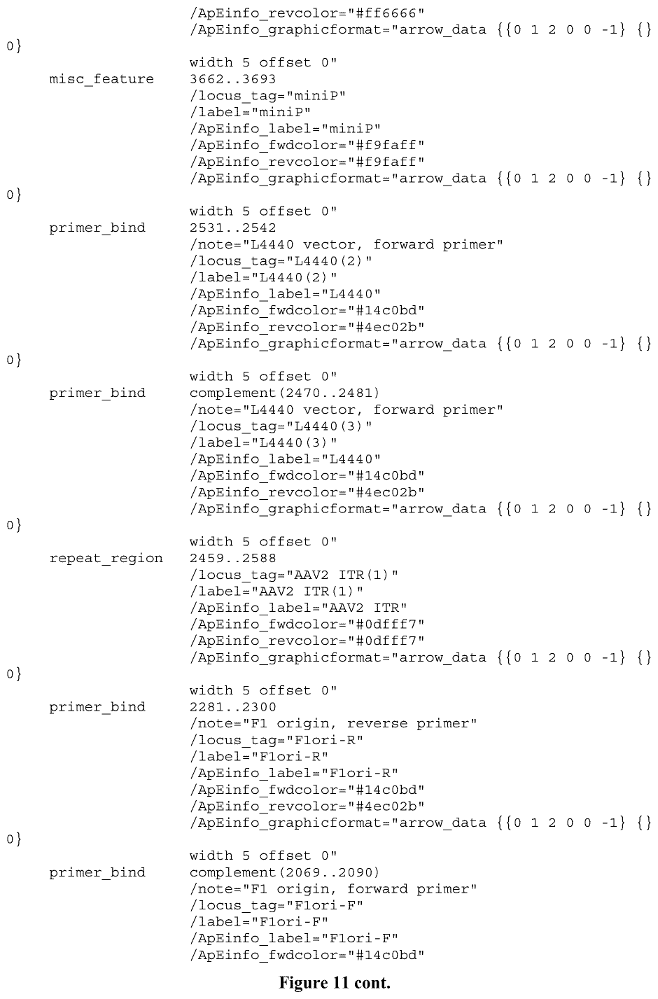

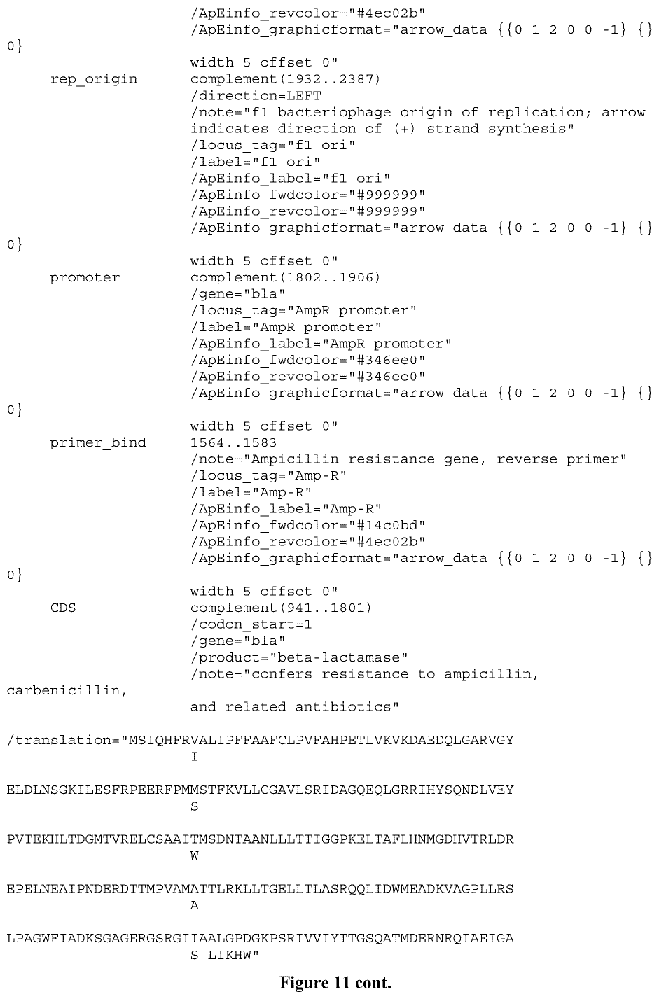

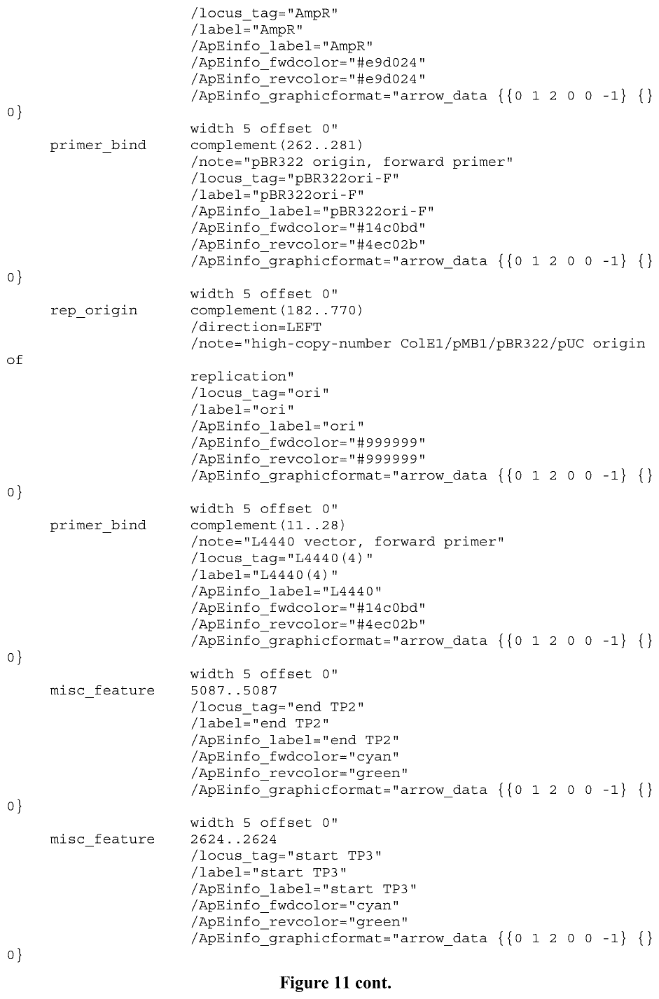

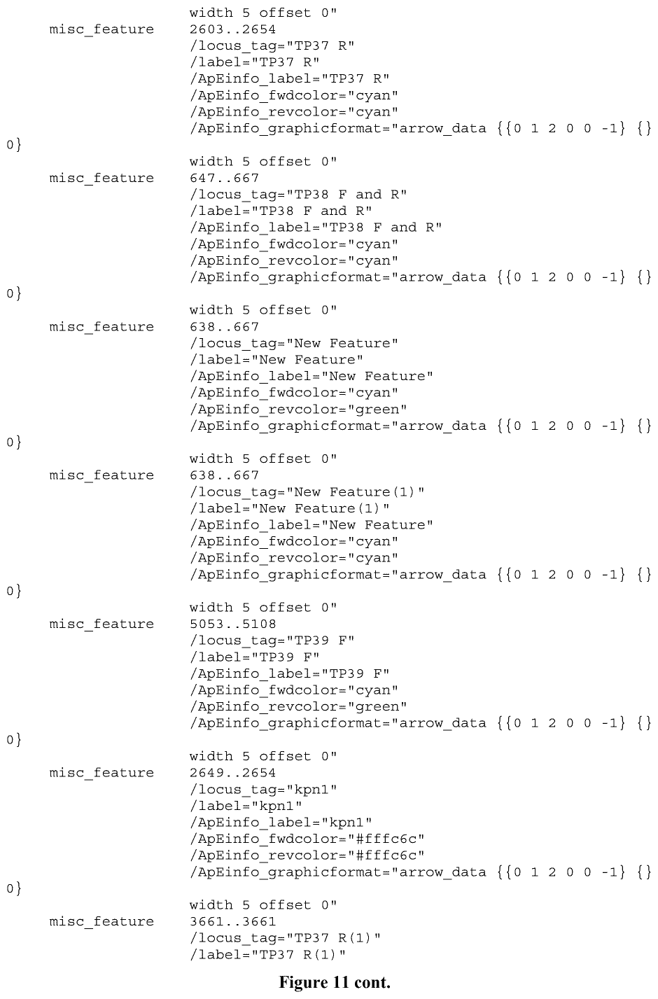

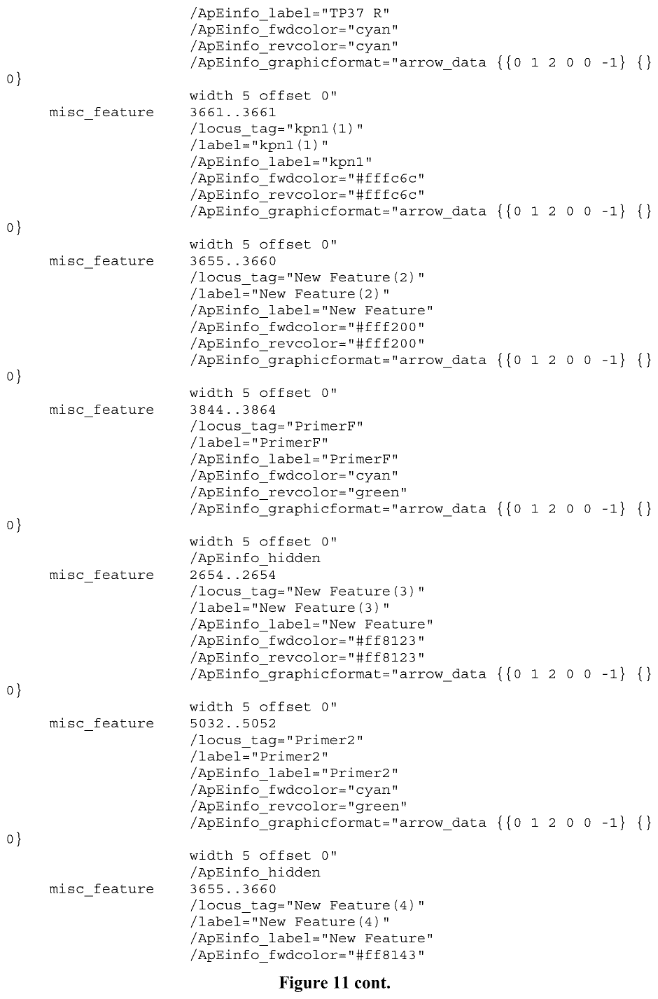

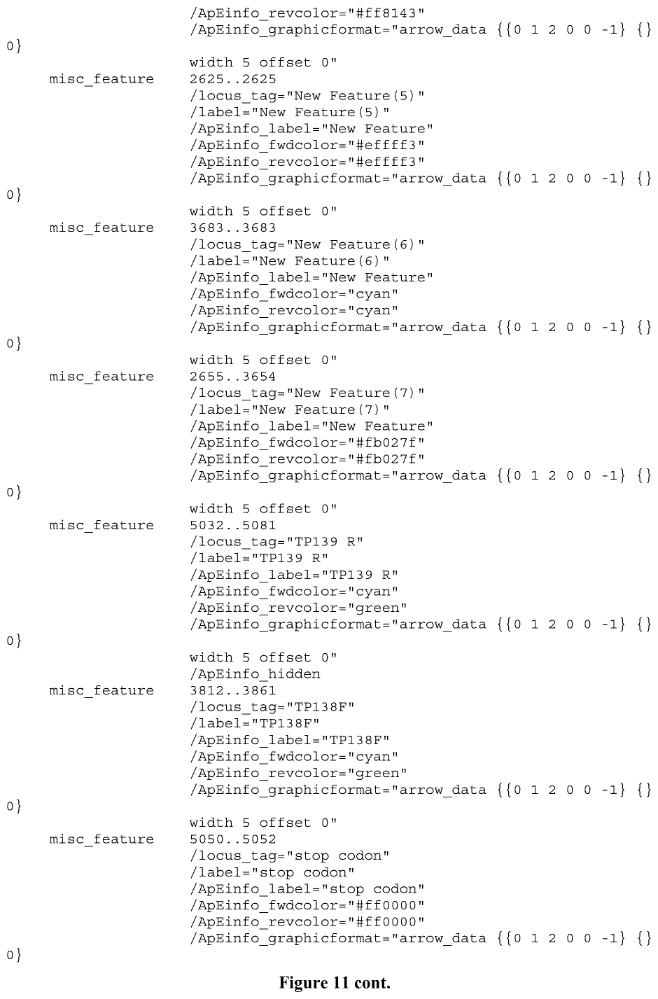

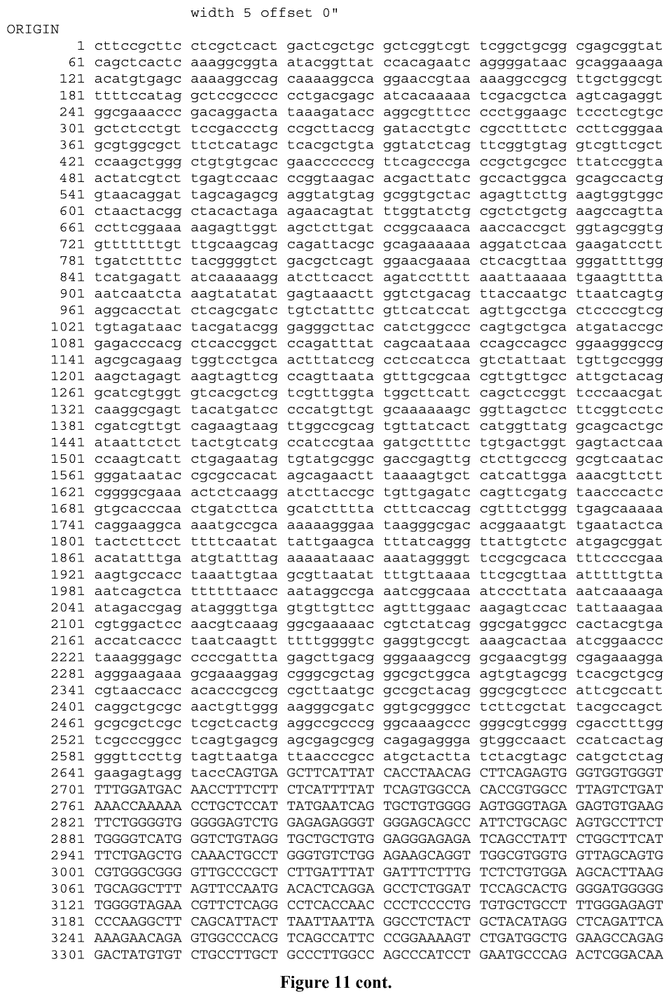

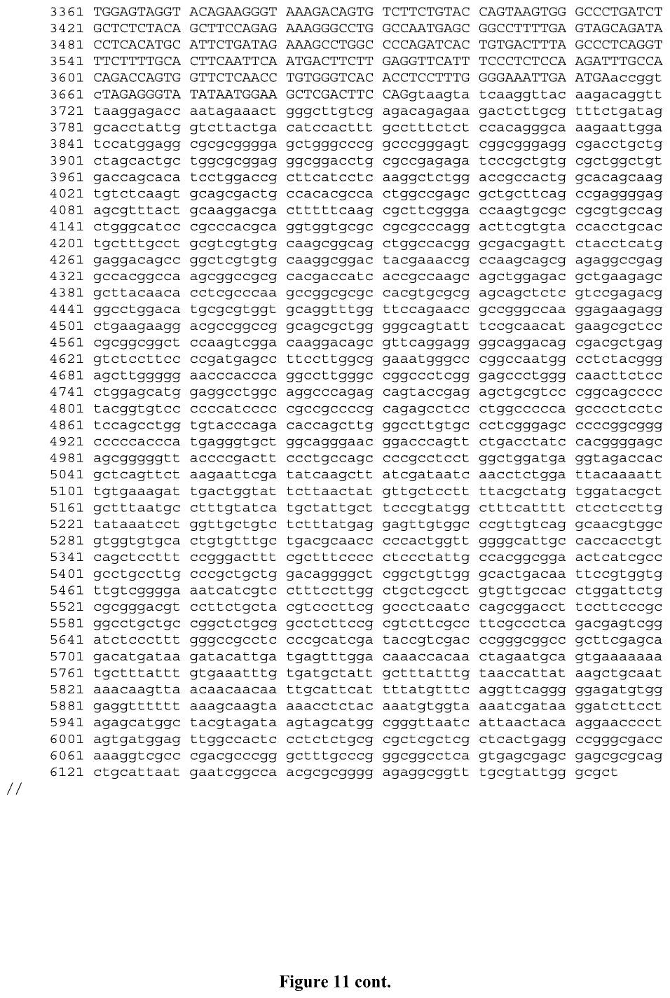

Sequence of the ChatE:
SEQ ID NO: 1 depicts the nucleotide sequence of the enhancer disclosed herein:
CAGTGAGCTTCATTATCACCTAACAGCTTCAGAGTGGGTGGTGGGTTT
TGGATGACAACCTTTCTTCTCATTTTATTCAGTGGCCACACCGTGGCCTTAGTCTG
ATAAACCAAAAACCTGCTCCATTATGAATCAGTGCTGTGGGGAGTGGGTAGAGA
GTGTGAAGTTCTGGGGTGGGGGAGTCTGGAGAGAGGGTGGGAGCAGCCATTCTG
CAGCAGTGCCTTCTTGGGGTCATGGGTCTGTAGGTGCTGCTGTGGAGGGAGAGAT
CAGCCTATTCTGGCTTCATTTCTGAGCTGCAAACTGCCTGGGTGTCTGGAGAAGC
AGGTTGGCGTGGTGGTTAGCAGTGCGTGGGCGGGGTTGCCCGCTCTTGATTTATG
ATTTCTTTGTCTCTGTGGAAGCACTTAAGTGCAGGCTTTAGTTCCAATGACACTCA
GGAGCCTCTGGATTCCAGCACTGGGGATGGGGGTGGGGTAGAACGTTCTCAGGC
CTCACCAACCCCTCCCCTGTGTGCTGCCTTTGGGAGAGTCCCAAGGCTTCAGCAT
TACTTAATTAATTAGGCCTCTACTGCTACATAGGCTCAGATTCAAAAGAACAGAG
TGGCCCACGTCAGCCATTCCCGGAAAAGTCTGATGGCTGGAAGCCAGAGGACTA
TGTGTCTGCCTTGCTGCCCTTGGCCAGCCCATCCTGAATGCCCAGACTCGGACAA
TGGAGTAGGTACAGAAGGGTAAAGACAGTGTCTTCTGTACCAGTAAGTGGGCCC
TGATCTGCTCTCTACAGCTTCCAGAGAAAGGGCCTGGCCAATGAGCGGCCTTTTG
AGTAGCAGATACCTCACATGCATTCTGATAGAAAGCCTGGCCCCAGATCACTGTG
ACTTTAGCCCTCAGGTTTCTTTTGCACTTCAATTCAATGACTTCTTGAGGTTCATT
TCCCTCTCCAAGATTTGCCACAGACCAGTGGTTCTCAACCTGTGGGTCACACCTC
CTTTGGGGAAATTGAATGA
SEQ ID NO: 2 depicts the nucleotide sequence of the ampicillin resistance gene
disclosed herein:
ATGAGTATTCAACATTTCCGTGTCGCCCTTATTCCCTTTTTTGCGGCATT
TTGCCTTCCTGTTTTTGCTCACCCAGAAACGCTGGTGAAAGTAAAAGATGCTGAA
GATCAGTTGGGTGCACGAGTGGGTTACATCGAACTGGATCTCAACAGCGGTAAG
ATCCTTGAGAGTTTTCGCCCCGAAGAACGTTTTCCAATGATGAGCACTTTTAAAG
TTCTGCTATGTGGCGCGGTATTATCCCGTATTGACGCCGGGCAAGAGCAACTCGG
TCGCCGCATACACTATTCTCAGAATGACTTGGTTGAGTACTCACCAGTCACAGAA
AAGCATCTTACGGATGGCATGACAGTAAGAGAATTATGCAGTGCTGCCATAACC
ATGAGTGATAACACTGCGGCCAACTTACTTCTGACAACGATCGGAGGACCGAAG
GAGCTAACCGCTTTTTTGCACAACATGGGGGATCATGTAACTCGCCTTGATCGTT
GGGAACCGGAGCTGAATGAAGCCATACCAAACGACGAGCGTGACACCACGATG
CCTGTAGCAATGGCAACAACGTTGCGCAAACTATTAACTGGCGAACTACTTACTC
TAGCTTCCCGGCAACAATTAATAGACTGGATGGAGGCGGATAAAGTTGCAGGAC
CACTTCTGCGCTCGGCCCTTCCGGCTGGCTGGTTTATTGCTGATAAATCTGGAGC
CGGTGAGCGTGGGTCTCGCGGTATCATTGCAGCACTGGGGCCAGATGGTAAGCC
CTCCCGTATCGTAGTTATCTACACGACGGGGAGTCAGGCAACTATGGATGAACG
AAATAGACAGATCGCTGAGATAGGTGCCTCACTGATTAAGCATTGGTAA
SEQ ID NO: 3 depicts the amino acid sequence encoded by the ampicillin
resistance gene disclosed herein:
MSIQHFRVALIPFFAAFCLPVFAHPETLVKVKDAEDQLGARVGYIELDLNS
GKILESFRPEERFPMMSTFKVLLCGAVLSRIDAGQEQLGRRIHYSQNDLVEYSPVTEK
HLTDGMTVRELCSAAITMSDNTAANLLLTTIGGPKELTAFLHNMGDHVTRLDRWEP
ELNEAIPNDERDTTMPVAMATTLRKLLTGELLTLASRQQLIDWMEADKVAGPLLRS
ALPAGWFIADKSGAGERGSRGIIAALGPDGKPSRIVVIYTTGSQATMDERNRQIAEIG
ASLIKHW-
SEQ ID NO: 4 depicts the nucleotide sequence of an exemplary AAV2 ITR
disclosed herein:
CTGCGCGCTCGCTCGCTCACTGAGGCCGCCCGGGCAAAGCCCGGGCGT
CGGGCGACCTTTGGTCGCCCGGCCTCAGTGAGCGAGCGAGCGCGCAGAGAGGGA
GTGGCCAACTCCATCACTAGGGGTTCCT
SEQ ID NO: 5 depicts the nucleotide sequence of an exemplary AAV2 ITR
disclosed herein:
AGGAACCCCTAGTGATGGAGTTGGCCACTCCCTCTCTGCGCGCTCGCT
CGCTCACTGAGGCCGGGCGACCAAAGGTCGCCCGACGCCCGGGCTTTGCCCGGG
CGGCCTCAGTGAGCGAGCGAGCGCGCAG
SEQ ID NO: 6 depicts the nucleotide sequence of an exemplary promoter
disclosed herein:
TAGAGGGTATATAATGGAAGCTCGACTTCCAG
SEQ ID NO: 7 depicts the nucleotide sequence of an exemplary intron disclosed
herein:
GTAAGTATCAAGGTTACAAGACAGGTTTAAGGAGACCAATAGAAACT
GGGCTTGTCGAGACAGAGAAGACTCTTGCGTTTCTGATAGGCACCTATTGGTCTT
ACTGACATCCACTTTGCCTTTCTCTCCACAG
SEQ ID NO: 8 depicts the nucleotide sequence of an exemplary woodchuck
hepatitis virus post-transcriptional regulatory element disclosed herein:
AATCAACCTCTGGATTACAAAATTTGTGAAAGATTGACTGGTATTCTTA
ACTATGTTGCTCCTTTTACGCTATGTGGATACGCTGCTTTAATGCCTTTGTATCAT
GCTATTGCTTCCCGTATGGCTTTCATTTTCTCCTCCTTGTATAAATCCTGGTTGCTG
TCTCTTTATGAGGAGTTGTGGCCCGTTGTCAGGCAACGTGGCGTGGTGTGCACTG
TGTTTGCTGACGCAACCCCCACTGGTTGGGGCATTGCCACCACCTGTCAGCTCCT
TTCCGGGACTTTCGCTTTCCCCCTCCCTATTGCCACGGCGGAACTCATCGCCGCCT
GCCTTGCCCGCTGCTGGACAGGGGCTCGGCTGTTGGGCACTGACAATTCCGTGGT
GTTGTCGGGGAAATCATCGTCCTTTCCTTGGCTGCTCGCCTGTGTTGCCACCTGGA
TTCTGCGCGGGACGTCCTTCTGCTACGTCCCTTCGGCCCTCAATCCAGCGGACCTT
CCTTCCCGCGGCCTGCTGCCGGCTCTGCGGCCTCTTCCGCGTCTTCGCCTTCGCCC
TCAGACGAGTCGGATCTCCCTTTGGGCCGCCTCCCCGC
SEQ ID NO: 9 depicts the nucleotide sequence of an exemplary polyA sequence
disclosed herein:
TAAGATACATTGATGAGTTTGGACAAACCACAACTAGAATGCAGTGAA
AAAAATGCTTTATTTGTGAAATTTGTGATGCTATTGCTTTATTTGTAACCATTATA
AGCTGCAATAAACAAGTT
SEQ ID NO: 10 as provided in FIG. 10 depicts the nucleotide sequence of
AAV-Isl1. Among other features, SEQ ID NO: 10 includes WPRE (4912-5500); Factor Xa
recognition and cleavage site (5383-5394, complementary strand); SV40 polyadenylation
signal (5543-5664); Isl1-ORF (3844-4890); AAV2 ITR (5826-5955 and 2459-2588); ChatE
enhancer (2655-3654); chimeric intron (3594-3826); AmpR promoter (1802-1906,
complementary strand); AmpR (941-1801, complementary strand).
SEQ ID NO: 11 as provided in FIG. 11 depicts the nucleotide sequence of
AAV-Lhx3. Among other features, SEQ ID NO: 11 includes WPRE (5077-5665); Factor Xa
recognition and cleavage site (5548-5559, complementary strand); SV40 polyadenylation
signal (5708-5829); Lhx3-ORF (3844-5052); AAV2 ITR (5991-6120 and 2459-2588); ChatE
enhancer (2655-3654); chimeric intron (3694-3826); AmpR promoter (1802-1906,
complementary strand); AmpR (941-1801, complementary strand).
SEQ ID NO: 12 depicts the amino acid sequence of Phox2a:
MDYSYLNSYDSCVAAMEASAYGDFGACSQPGGFQYSPLRPAFPAAGPPC
PALGSSNCALGALRDHQPAPYSAVPYKFFPEPSGLHEKRKQRRIRTTFTSAQLKELER
VFAETHYPDIYTREELALKIDLTEARVQVWFQNRRAKFRKQERAASAKGAAGAAGA
KKGEARCSSEDDDSKESTCSPTPDSTASLPPPPAPGLASPRLSPSPLPVALGSGPGPGP
GPQPLKGALWAGVAGGGGGGPGAGAAELLKAWQPAESGPGPFSGVLSSFHRKPGP
ALKTNLF
Additional isoforms of Phox2a include NP_001412025.1, NP_001412026.1,
NP_001412027.1, XP_047282903.1, XP_054224755.1, accessible through the NCBI
database and which are incorporated by reference in their entireties.
SEQ ID NO: 13 depicts the amino acid sequence of Oct4 (Isoform 1):
MAGHLASDFAFSPPPGGGGDGPGGPEPGWVDPRTWLSFQGPPGGPGIGPG
VGPGSEVWGIPPCPPPYEFCGGMAYCGPQVGVGLVPQGGLETSQPEGEAGVGVESN
SDGASPEPCTVTPGAVKLEKEKLEQNPEESQDIKALQKELEQFAKLLKQKRITLGYTQ
ADVGLTLGVLFGKVFSQTTICRFEALQLSFKNMCKLRPLLQKWVEEADNNENLQEIC
KAETLVQARKRKRTSIENRVRGNLENLFLQCPKPTLQQISHIAQQLGLEKDVVRVWF
CNRRQKGKRSSSDYAQREDFEAAGSPFSGGPVSFPLAPGPHFGTPGYGSPHFTALYSS
VPFPEGEAFPPVSVTTLGSPMHSN
Additional isoforms of Oct4 include NP_001167002.1, NP_001272916.1,
NP_001272915.1, and NP_976034.4 accessible through the NCBI database and which are
incorporated by reference in their entireties.
SEQ ID NO: 14 depicts the amino acid sequence of Sox2:
MYNMMETELKPPGPQQTSGGGGGNSTAAAAGGNQKNSPDRVKRPMNAF
MVWSRGQRRKMAQENPKMHNSEISKRLGAEWKLLSETEKRPFIDEAKRLRALHMK
EHPDYKYRPRRKTKTLMKKDKYTLPGGLLAPGGNSMASGVGVGAGLGAGVNQRM
DSYAHMNGWSNGSYSMMQDQLGYPQHPGLNAHGAAQMQPMHRYDVSALQYNS
MTSSQTYMNGSPTYSMSYSQQGTPGMALGSMGSVVKSEASSSPPVVTSSSHSRAPC
QAGDLRDMISMYLPGAEVPEPAAPSRLHMSQHYQSGPVPGTAINGTLPLSHM

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

PHP.eB-AAVs were generated that demonstrate high blood-brain barrier penetrance and strong tropism for motor neurons in the spinal cord, where Isl1/Lhx3 expression controlled by a ChAT enhancer, so that Isl1/Lhx3 expression is limited to motor neurons (viruses are referred to hereafter as AAV-Isl1 and AAV-Lhx3). These viruses were injected into SOD1^{G93A} ALS model mice and measured protein aggregation phenotypes associated with ALS.

As controls, protein aggregation phenotypes were evaluated in uninjected mice, mice injected with AAVs encoding mCherry, a fluorescent protein with no function in the cell, and mice injected with AAVs encoding Phox2a, a transcription factor important for cranial motor neuron identity.

### Example 1—AAV-Isl1, AAV-Lhx3 Lead to Reduction in p62 Aggregation in 45 Day Old ALS Mice

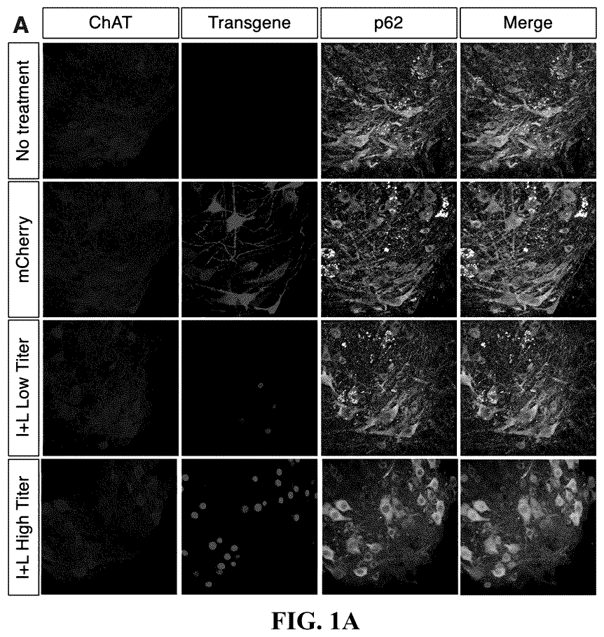

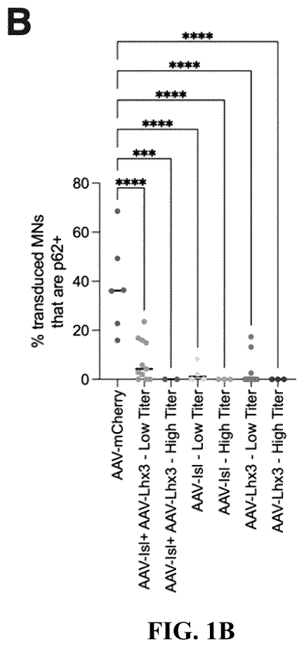

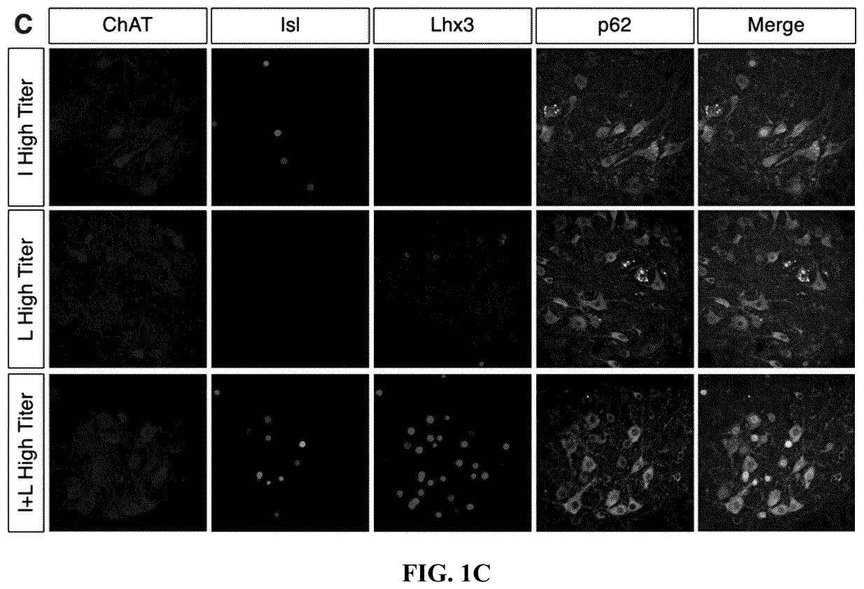

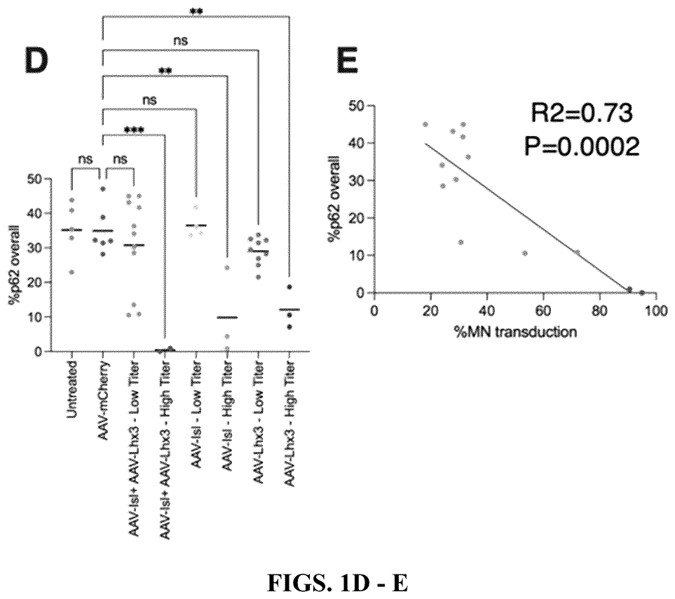

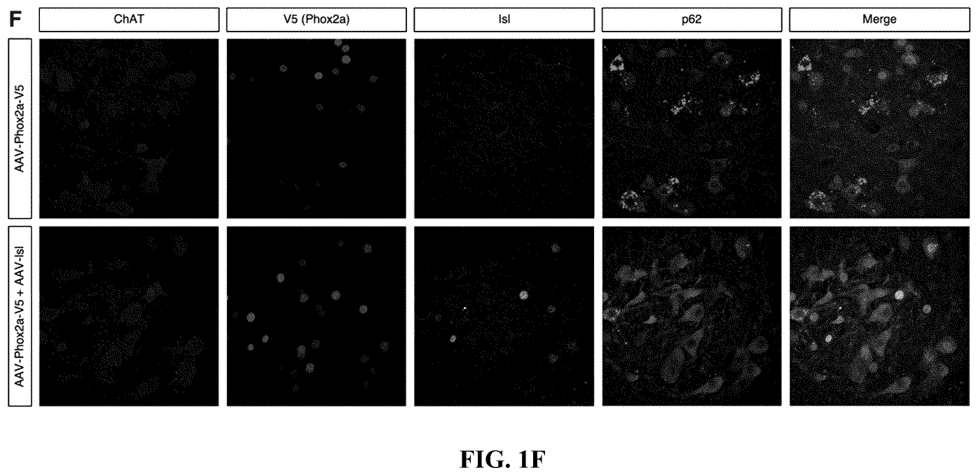

The aggregation of the autophagy-related protein p62 was measured in spinal motor neurons, one of the earliest histological phenotypes to appear in SOD1^{G93A} ALS mouse model. Mutations in SQSTM1, the gene encoding p62, can also cause ALS. Mice were treated at neonatal stage and p62 aggregation was measured at P45 (45 day old mice) (FIGS. 1A-G). p62 aggregation was compared in the following conditions:
a. Uninjected mice
b. Mice injected with “low” titer (3E+10 viral genome copies (gc)/virus/animal) of Isl1 alone, Lhx3 alone, and Isl1+Lhx3 combined
c. Mice injected with “high” titer (3E+11 viral genome copies (gc)/virus/animal) of Isl1 alone, Lhx3 alone, and Isl1+Lhx3 combined
d. Mice injected with mCherry
e. Mice injected with the cranial motor neuron transcription factor Phox2a

It was found that motor neurons expressing AAV-Isl1 and AAV-Lhx3 individually or in combination were protected from p62 aggregation, while motor neurons of uninjected mice, and motor neurons expressing AAV-mCherry or AAV-Phox2a showed similar levels of p62 aggregation. In addition, it was found that a significant, dose-dependent reduction in the overall level of p62 in the spinal cords of mice injected with AAV-Isl1 and AAV-Lhx3 in combination (AAV-Isl+AAV-Lhx3). In mice injected with high titers of AAV-Isl+AAV-Lhx3 p62 aggregation was almost completely undetectable (FIG. 1A-E). These data suggest that re-expression of Isl1+Lhx3 together in neonatal mice results in reduced p62 aggregation in SOD1^{G93A} ALS mice, and that these effects are specific to the spinal motor neuron factors Isl1 and Lhx3 but not cranial motor neuron factors such as Phox2a (FIG. 1F-G).

### Example 2—AAV-Isl1, AAV-Lhx3 Lead to Reduction in p62 Aggregation in 75 Day Old ALS Mice

While p62 aggregation is an important ALS-relevant phenotype that reflects deficits in autophagy and proteostasis, and tracks directly with motor neuron degeneration, the studies were expanded to explore the impact of Isl1 and Lhx3 re-expression on additional phenotypes that appear later in the course of the disease. Tissues were collected from a small cohort of AAV-mCherry (n=3) and AAV-Isl1+AAV-Lhx3-treated animals (n=4) at P75 (75 day old mice) and performed immunostaining for p62. For these studies all animals were treated with “low” titer viruses (3E+10 viral particles/animal).

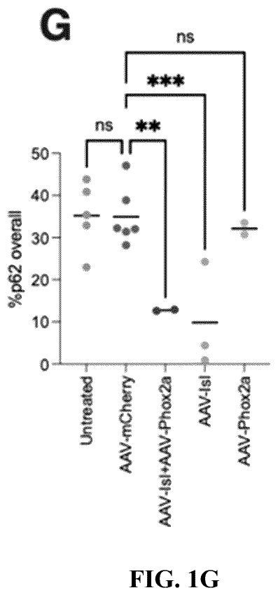

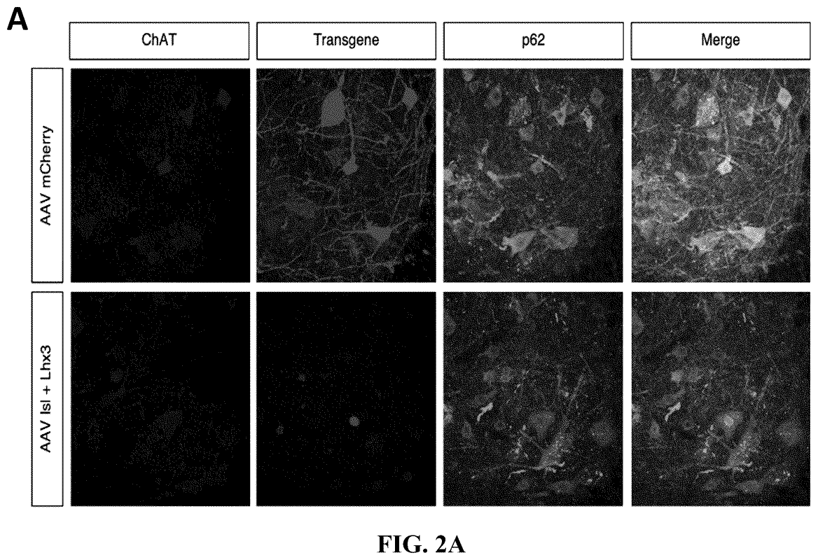

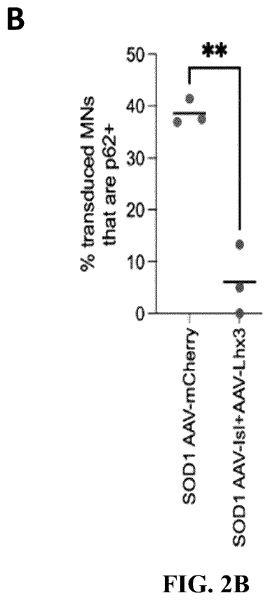

It was found that similarly to P45, AAV-Isl1 and/or AAV-Lhx3-transduced motor neurons at P75 were significantly less likely than AAV-mCherry-transduced motor neurons to show p62 aggregation (FIG. 2A-B).

### Example 3—AAV-Isl, AAV-Lhx3 Lead to Reduction in SOD1 Aggregation in 75 Day Old ALS Mice

Next, the effects of Isl1 and Lhx3 re-expression on SOD1 aggregation were investigated using an antibody that is specific for mutant human SOD1. Insoluble mutant SOD1 species are directly linked to motor neuron degeneration in this mouse model, and SOD1 aggregates have been shown to co-localize with p62 aggregates in both mouse and human patient tissue (Trist et al. 2022). Inventors performed SOD1 staining in the same cohort of animals used in Example 2.

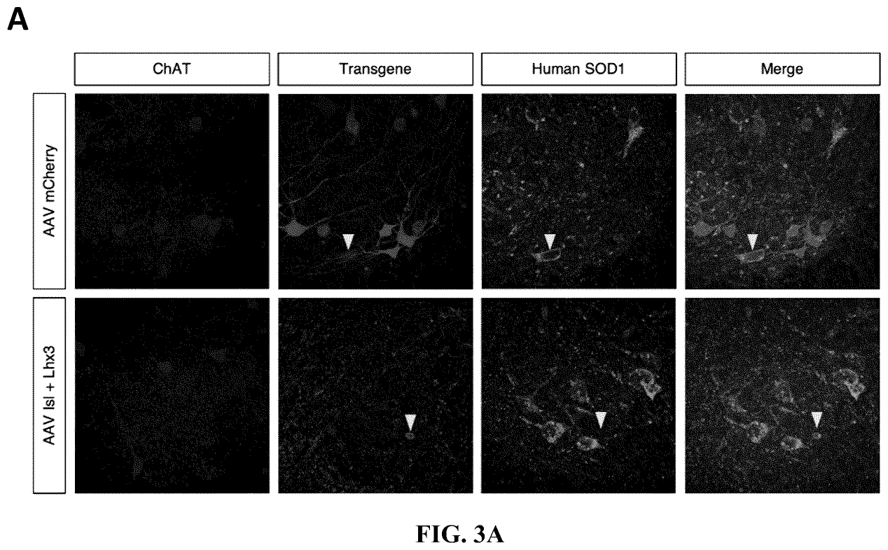

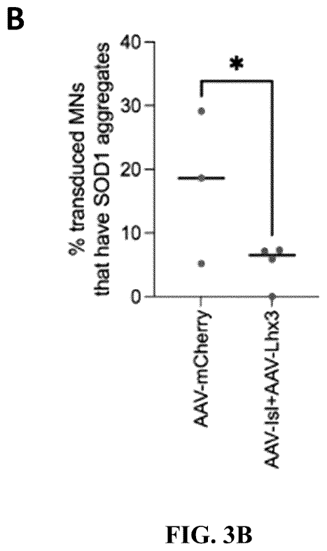

Similar to p62, it was found that AAV-Isl1+AAV-Lhx3-transduced cells were significantly less likely than AAV-mCherry-transduced cells to show SOD1 aggregation (FIG. 3A-B), suggesting that Isl1 and Lhx3 re-expression are impacting multiple disease-relevant proteinopathies.

### Example 4—AAV-Isl, AAV-Lhx3 Lead to Reduction in Microgliosis in 75 Day Old ALS Mice

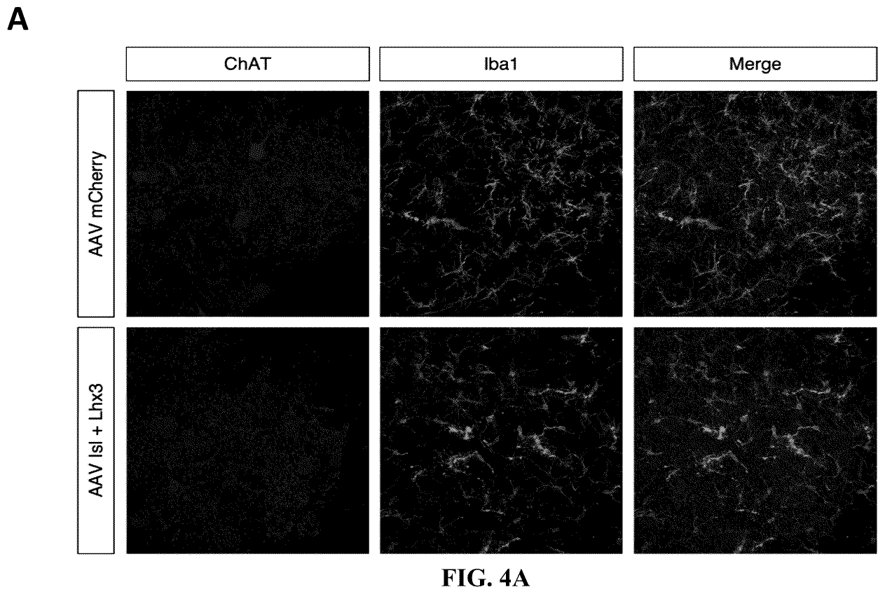

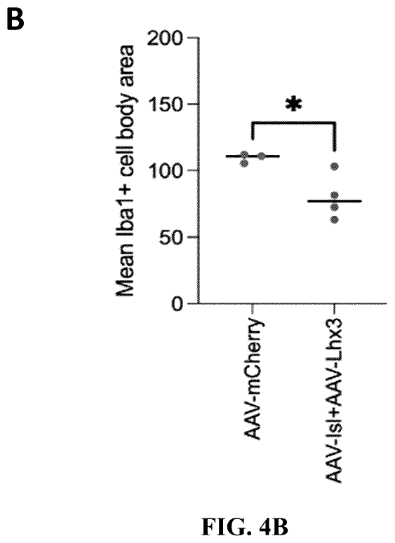

The effects of Isl1 and Lhx3 re-expression on neuroinflammation were evaluated by staining for the microglial marker Iba1 and a significant reduction in Iba1 levels was observed in the vicinity of vulnerable motor neurons in the L4-L5 region of the spinal cord (FIG. 4A-B). To do this, Iba1 staining was performed in the same cohort of animals used in Experiment 2.

It was found that Iba1 staining was lower in Isl1+Lhx3 injected animals compared to mCherry injected animals. These preliminary results suggest that, by addressing primary, motor neuron-specific pathologies, Isl1 and Lhx3 re-expression may also be ameliorating downstream secondary pathologies such as neuroinflammation.

### Example 5—Postnatal Re-Expression of Motor Neuron Programming Factors Suppresses ALS Proteinopathies

ALS is characterized by progressive motor neuron degeneration, typically leading to paralysis and death 2-5 years after diagnosis 1. Neurodegeneration in ALS is both cell type-specific and age dependent, affecting only certain subtypes of motor neurons with a typical age of onset of 55-75 years. Most disease-causing mutations in the ˜10% of patients that have inherited forms of ALS lead to protein destabilization, misfolding, mislocalization, or aggregation, and insoluble, ubiquitinated protein aggregates can be detected in the motor cortex and spinal cord of nearly all post-mortem patient samples 2. Spinal motor neurons are intrinsically less capable of handling proteostatic stress than other neuronal cell types 3, including cranial motor neurons that are generally spared until later stages of the disease, pointing to a causative role for proteostatic stress in the cell specificity of ALS pathology 4. Furthermore, temporal profiling of gene expression in mouse spinal motor neurons has shown that genes involved in protein metabolism, protein folding, and the clearance of misfolded proteins are downregulated over the course of motor neuron maturation, suggesting that adult motor neurons may handle misfolded proteins less efficiently than nascent motor neurons 5. Without intending to be bound by any particular theory, it is hypothesized that reverting postnatal spinal motor neurons to a more immature state may enhance their ability to buffer proteostatic stress and reset their resistance to ALS pathologies.

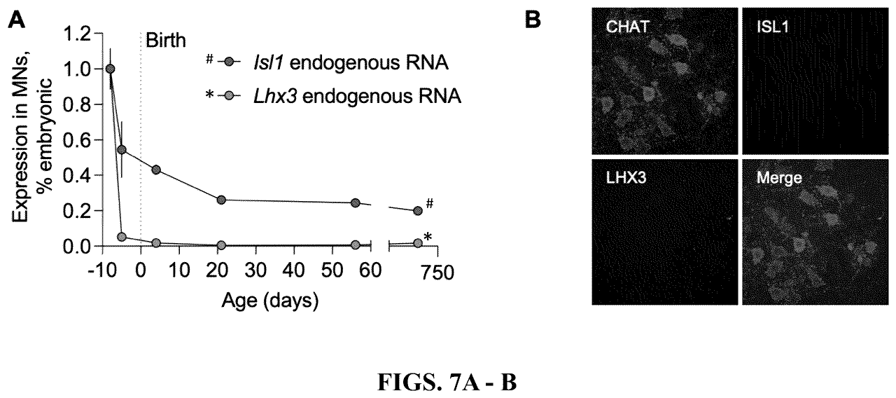

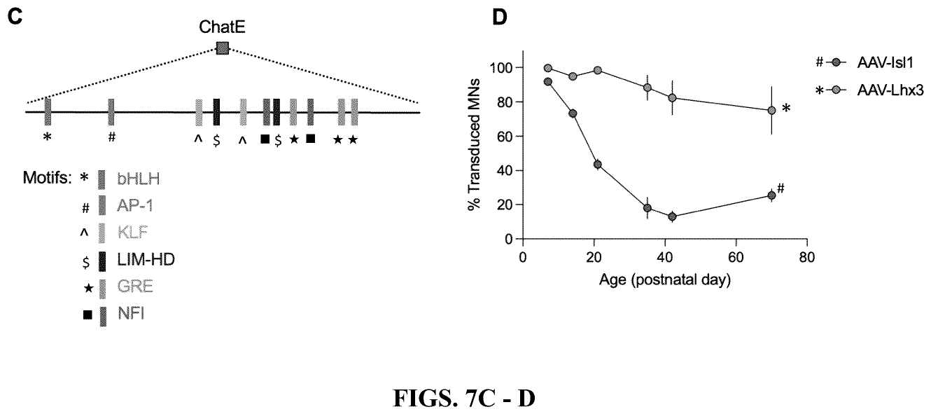

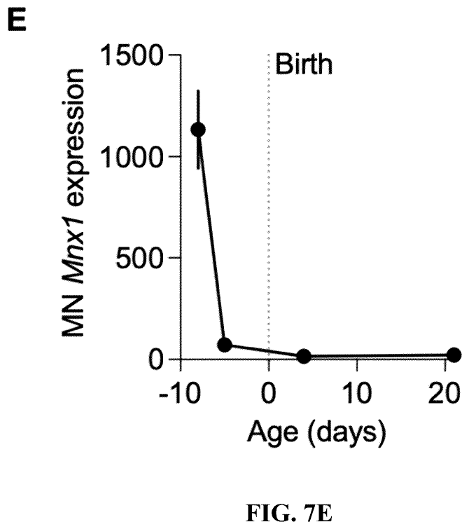

It has previously been shown that the majority of the genes induced in nascent motor neurons are direct targets of two transcription factors: ISL1 and LHX3 6-8. While essential for spinal motor neuron specification, both factors are downregulated in postnatal motor neurons (FIG. 7A,B) 5. ISL1 and LHX3 function as motor neuron selector transcription factors and can reprogram neural progenitors, pluripotent stem cells, or adult skin fibroblasts into immature spinal motor neurons 7,9,10, making them strong candidates for factors that could re-activate an immature gene expression state in adult motor neurons in vivo.

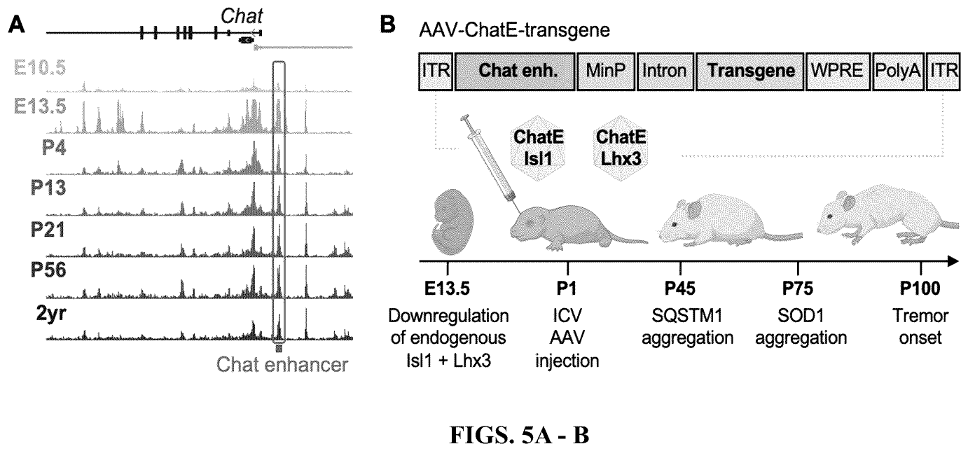

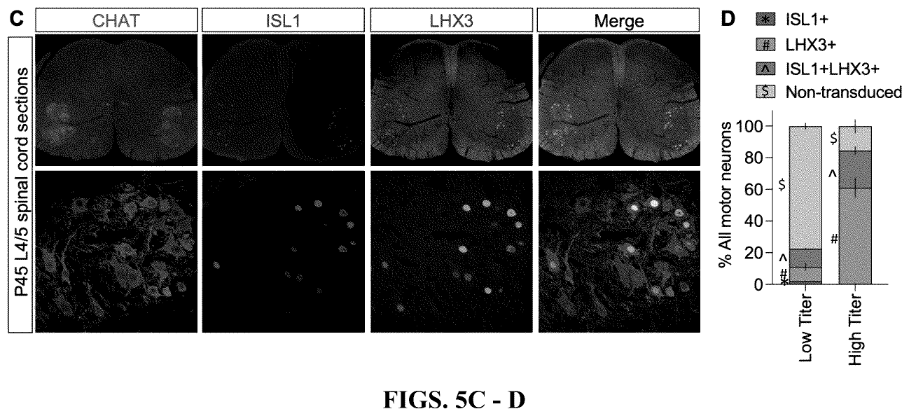

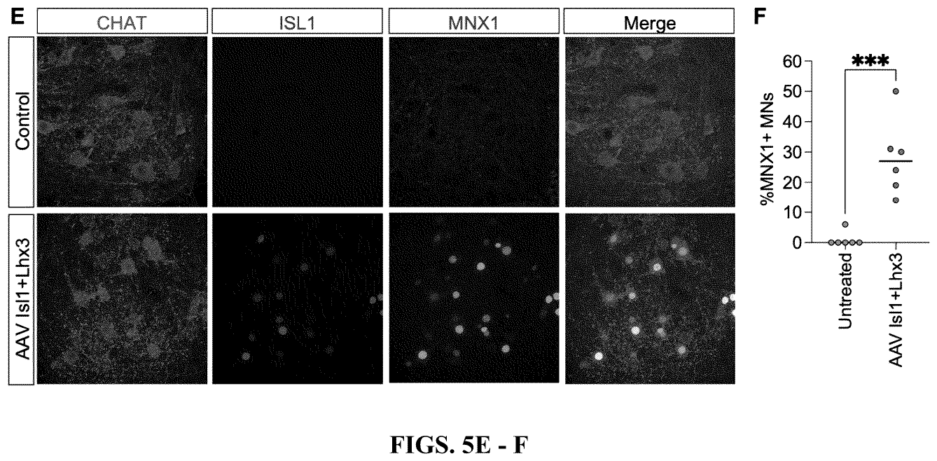

To test this idea, adeno-associated viruses (AAVs) that drive skeletal motor neuron specific expression of Isl1 and Lhx3 in postnatal mouse spinal cords were generated. Cell type specific expression was achieved by using a 1000 bp enhancer 3 Kb upstream of the Chat gene (ChatE; FIG. 5A). This enhancer was identified as a putative motor neuron regulatory element in a temporal ATAC-seq dataset 5. It was chosen because it is continuously accessible at all ages (FIG. 1A), and contains binding sites for key transcription factors controlling motor neuron specification and maturation (FIG. 7C) 5,7,8. AAV-Isl1 and AAV-Lhx3 were co-administered at a 1:1 ratio by intracerebroventricular injection into neonatal mice at postnatal day 1 (P1; FIG. 5B), yielding strong and specific motor neuron expression by immunohistochemistry (IHC; FIG. 5C), while endogenous ISL1 and LHX3 expression remained undetectable in uninjected control animals or animals transduced with AAV-mCherry (Extended data FIG. 1B). At doses above 3E+11 total viral genomes (vg) per animal, >90% motor neuron transduction was observed with near-complete overlap between ISL1 and LHX3 positive cells at early post-injection timepoints (FIG. 5C), though AAV-mediated ISL1 expression decreased with time (FIG. 7D). By P45, 84% of motor neurons retained expression of AAV-driven LHX3, with 61% expressing LHX3 alone and 23% co-expressing ISL1 and LHX3 (FIG. 5D).

To evaluate whether heterochronically re-expressed ISL1 and LHX3 could reactivate their embryonic targets, lumbar spinal cord tissue collected at P45 was stained for MNX1 (HB9), a gene that is specifically expressed in nascent motor neurons and downregulated after birth (FIG. 7E). Accordingly, it was found that MNX1 was virtually undetectable in untreated or control AAV-treated animals (FIG. 5E). Treatment with AAV-Isl1+AAV-Lhx3 led to significant upregulation of MNX1 (p=0.0004; FIG. 1E-F), with up to ˜90% of AAV-Isl+ motor neurons exhibiting MNX1 expression (FIG. 5E).

It was then asked whether ISL1 and LHX3 re-expression could attenuate disease-relevant proteinopathies in the SOD1^{G93A} transgenic mouse model of ALS. This model recapitulates many biochemical and behavioral features of the disease, including early and severe deficits in the processing of misfolded protein in vulnerable spinal motor neuron populations, followed by neuromuscular junction denervation, motor neuron degeneration, neuroinflammation, and clinical symptoms that progress swiftly until endstage at ˜P157.

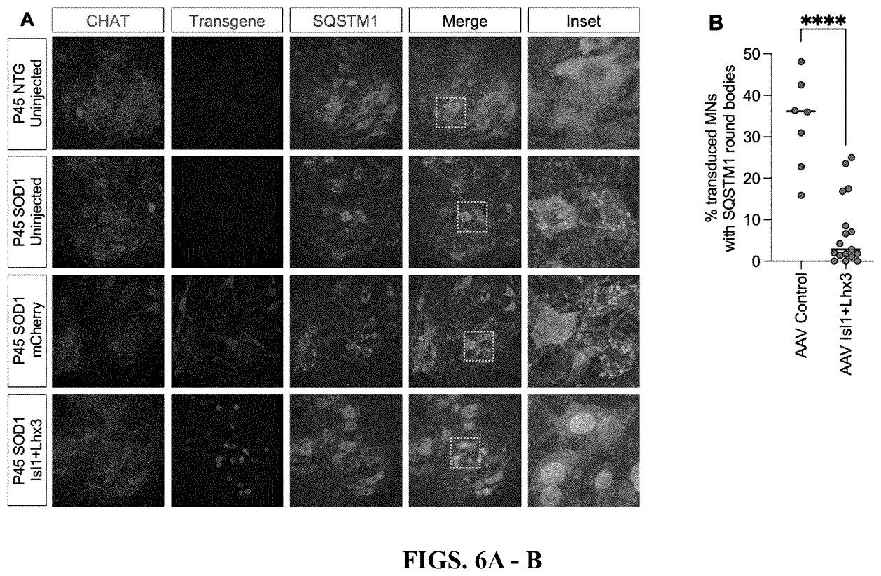

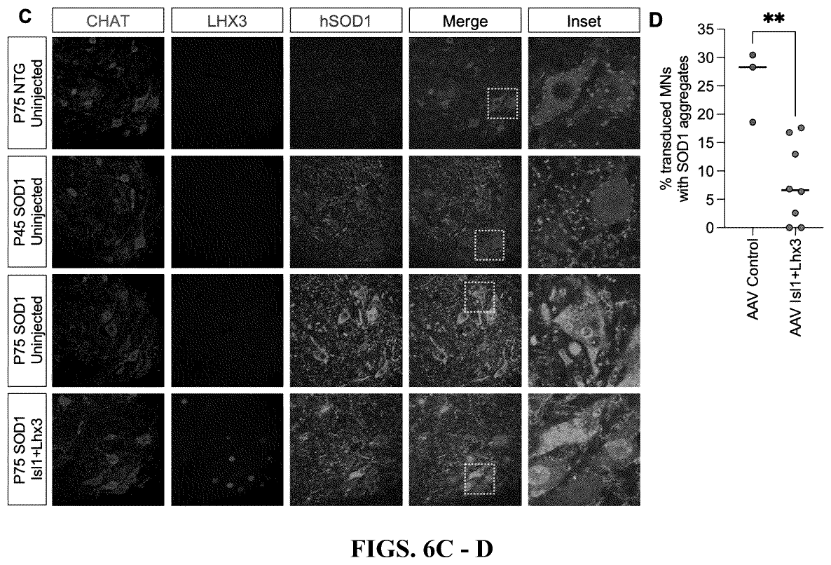

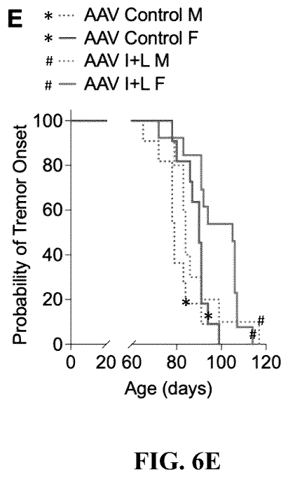

One of the earliest histological markers of protein dyshomeostasis in this model is SQSTM1, a key component of both the ubiquitin-proteasome system and the macroautophagy pathway that regulates lysosomal protein degradation 11. Large, round aggregates of SQSTM1 (termed “round bodies” 12) are detectable in the cytoplasm of lumbar motor neurons as early as P35 13, and by P45, they can be found in nearly one third of motor neurons (see FIG. 6A). Highlighting the relationship between proteostatic defects and clinical severity, motor neuron-specific knockout of the autophagy gene Atg7 in SOD1^{G93A} mice dramatically enhances SQSTM1 aggregation and hastens the onset of clinical symptoms 12. Overexpression of SQSTM1 itself promotes the cytoplasmic aggregation of mutant SOD1 and accelerates onset in SOD1^{H46R} mice 14. Importantly, SQSTM1-positive aggregates that are co-positive for TDP43 and other ubiquitinated proteins are commonly observed in postmortem spinal cord tissues from both familial and sporadic ALS patients 15, and mutations in SQSTM1 have been causally linked to ALS 16, reinforcing the translational relevance of SQSTM1 dysregulation.

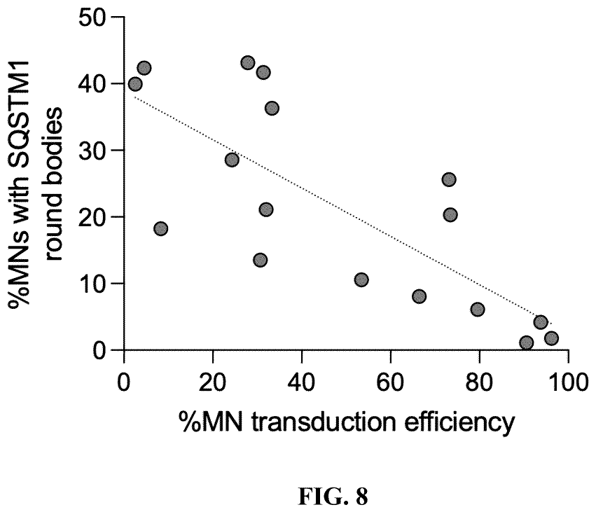

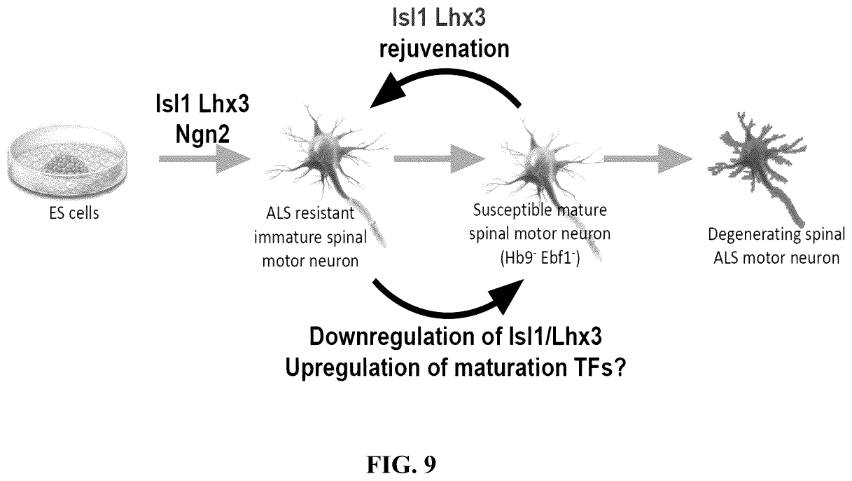

It was found that AAV-Isl1+AAV-Lhx3 administered at P1 reduced the incidence of SQSTM1-positive round bodies by >12-fold in transduced motor neurons (from 36.18% to 2.86%) at P45 compared to AAV-mCherry (p<0.0001, FIG. 6A,B). The effects of AAV-Isl1+AAV-Lhx3 across a range of viral doses (6.37E+10-3.69E+11 vg/animal) on the frequency of SQSTM1 round body-positive motor neurons were then compared and it was found that the total number of motor neurons per hemisection that exhibited SQSTM1 pathology was strongly correlated with AAV transduction efficiency (R²=0.5910, p=0.0003; FIG. 8). At transduction efficiencies greater than −80%, round body formation was almost completely abrogated (FIG. 8A,B and FIG. 8). Meanwhile, AAV-Isl1+AAV-Lhx3 treatment did not reduce the average number of motor neurons per hemisection (data not shown), nor did it prevent the expression of SQSTM1 (FIG. 8A), suggesting that the effect of ISL1 and LHX3 re-expression on SQSTM1 round body formation was a direct reflection of improved proteostatic conditions.

SQSTM1 has been shown to selectively bind mutant SOD1 and can actively sequester it into cytoplasmic inclusions of ubiquitinated proteins that intensify over time in spinal motor neurons in SOD1 mutant mouse models 13,14. To investigate the effects of ISL1 and LHX3 re-expression on this process, the incidence of SOD1-positive aggregates in spinal motor neurons was quantified using an antibody specific for the human transgene. Compared to SQSTM1 aggregates, which were pronounced by P45, it was observed that cytoplasmic SOD1 aggregates were more readily detectable at later stages of the disease (FIG. 8C), consistent with previous reports 17. By P75, when SOD1 aggregation was severe in untreated or AAV-mCherry-treated animals, it was found that AAV-Isl1+AAV-Lhx3 treatment reduced the formation of SOD1+ aggregates by >3-fold in transduced motor neurons (p=0.0042; FIG. 8D). These findings suggest that ISL1 and LHX3 re-expression in mature motor neurons can prevent a cascade of proteostatic deficits that drives disease progression.

Given that the exacerbation of SQSTM1-linked protein dyshomeostasis accelerates the onset of clinical symptoms in SOD1 mutant mice 12,14, it was then asked whether AAV-Isl1+AAV-Lhx3 treatment could delay symptom onset. For these experiments 7.56E+10 vg/animal was used, one of the lowest doses that was tested that still yielded significant reduction of SQSTM1 aggregation at P45. SOD1^{G93A} animals were treated with AAV-mCherry or AAV-Isl1+AAV-Lhx3 at P1 and monitored them over time for the appearance of fine hindlimb tremors, which marks the onset of clinical symptoms in this model 18. It was found that AAV-Isl1+AAV-Lhx3 treatment significantly delayed the appearance of tremors by 15 days in females from P90 to P105 (p=0.0036; FIG. 8E). There was a similar trend in males, though it was not significant (median delay=5 days from P79 to P84; p=0.1138; FIG. 8E), perhaps because male SOD1^{G93A} mice generally show more rapid progression of clinical phenotypes than females 19. In parallel studies, it was observed that motor neuron transduction efficiency under these conditions was ˜30% at P45 (FIG. 5D and FIG. 7D), and given the dose-dependency of the effects of AAV-Isl1+AAV-Lhx3 on SQSTM1 aggregation, these findings raise the possibility that improving the stability of transgene expression in future studies could yield additional phenotypic benefits.

This study demonstrates that development of highly targeted reprogramming approaches to rejuvenate specific adult neuron types and suppress adult-onset neurodegenerative diseases can be possible. In contrast to methods that rejuvenate cells by globally reverting their epigenetic landscape to a more stem-like state through the overexpression of general pluripotency factors (Oct4, Sox2, and Klf4), the approach described herein combines native motor neuron factors with a motor neuron-specific expression system to precisely alter the transcriptional state of the cells most vulnerable to disease. It was found that expression of ISL1 and LHX3 in postnatal motor neurons reactivates their embryonic target gene MNX1. Further analysis of gene expression and accessibility can be used to establish the extent to which heterochronic ISL1 and LHX3 can revert postnatal motor neurons to a more immature state. Functionally, it was found that ISL1 and LHX3 re-expression at an early postnatal stage could prevent key histological and clinical phenotypes in a mouse model of ALS, a disease of aging that results in profound spinal motor neuron degeneration. In summary, the work described herein demonstrates that heterochronic re-expression of embryonic selector transcription factors in postnatal animals is an effective therapeutic strategy that can cell-autonomously ameliorate protein dyshomeostasis in adult-onset neurodegenerative disease.

### Methods

Cloning pAAVs. ChatE, Isl1, and Lhx3 were Gibson cloned into an AAV2 backbone (pAAV). The pAAV backbone contained an AAV2 ITR, a cloning site, a mini promoter (TAGAGGGTATATAATGGAAGCTCGACTTCCAG) (SEQ ID NO: 6), chimeric intron, mCherry, WPRE, SV40 poly(A) signal, and the second AAV2 ITR. This pAAV was linearized by digesting at the cloning site with Kpn1. ChatE was amplified from genomic DNA of wildtype C57BL6/J mice with primers containing ˜28 bps of homology to the pAAV backbone, and inserted into linearized pAAV with Gibson cloning. This pAAV plasmid containing the ChatE and mCherry was then digested with BamHI and EcoRV to remove the mCherry and replace it with either Isl1 or Lhx3. Isl1 and Lhx3 cDNAs were amplified from other plasmids with ˜28 bps of homology to pAAV-ChatE backbone for Gibson cloning. All digestion sites were recreated after Gibson insertions to make it easy to switch either the enhancer or the cargo in the pAAV plasmid.

AAV production. AAVs were generated and titered by following the protocol in Challis et al ¹. No meaningful changes were made to the protocol. The PHP.eB cap plasmid and pHelper plasmid were used in combination with one of the three pAAV plasmids (mCherry, Isl1, or Lhx3) to generate each virus independently. If the titer of the viral preparations fell below 2E+12 vg/mL, they were respun through an Amicon filtration device to reduce overall volume and increase the final concentration to the desired titer.

Animals. All mouse experimental procedures were approved by the Columbia University Medical Center Institutional Animal Care and Use Committee. All studies were performed in mice that were heterozygous for the mutant human SOD1G93A transgene on a C57BL/6J background (B6.Cg-Tg(SOD1*G93A)1Gur/J) and their non-transgenic littermates. Breeding pairs consisted of C57BL6/J females and SOD1G93A males purchased from Jackson laboratories where they were assessed for transgene copy number maintenance. Pups were genotyped at P0 and balanced into treatment groups by sex, genotype, and littermate status. Intracerebroventricular AAV injections were performed on cryoanesthetized pups at P1 using a 10 μL syringe (Hamilton 7653-01) outfitted with a 0.375-inch 32 gauge needle (Hamilton 7803-04). The needle was inserted at ⅖ths of the distance between the lambda suture and the middle of the eye at a depth of 3 mm, and up to 6 μL of virus was injected per animal. For analysis of clinical onset, animals were monitored for the appearance of hindlimb tremors on a daily basis beginning at P40. Tremors were assessed qualitatively as per ² by an experimenter blind to the genotype and treatment of the animals.

Immunohistochemistry. For all immunohistochemical analyses, tissues were collected by transcardial perfusion with 10 mL ice-cold phosphate-buffered saline, followed by 40 mL of 4% paraformaldehyde in 0.1M phosphate buffer. The skull and spine were gross dissected and post-fixed for 18-24 h in 4% paraformaldehyde. The L4-L5 region of the spinal cord was isolated by cutting the spinal column at the T10 and S1 vertebrae, followed by laminectomy and transection of the spinal cord at the L3 and L6 roots. Spinal cord segments were stored at 4° in PBS containing 0.1% sodium azide until they were sectioned at 70μ using a vibratome fitted with a disposable blade (Leica). Immunostaining was performed in floating sections using antibodies against CHAT (1:250, Millipore AB144P), ISL1 (1:10000, Thomas M. Jessell Laboratory, Columbia University), LHX3 (1:5000, Thomas M. Jessell Laboratory, Columbia University), MNX1 (1:15000, Thomas M. Jessell Laboratory, Columbia University), SQSTM1 (1:500, Abcam ab56416), or human SOD1 (1:250, R&D Systems MAG 3418). Sections were incubated for 18-24 h in primary antibody solution containing 1% bovine serum albumin, 0.4% Triton, and 0.1% sodium azide in TRIS-buffered saline. Sections were then incubated overnight in secondary antibody solution containing 0.4% Triton in TRIS-buffered saline using antibodies raised in donkey and conjugated to Alexa Fluor 405, 488, 594, or 647 (Thermo Fisher). Sections were washed, mounted on slides, and coverslipped with Fluoromount-G (Southern Biotech).

Confocal Microscopy. Immunostained spinal cord sections were visualized at 5× using an epifluorescent microscope (Zeiss Axioscope) and at 40× using a confocal microscope with an oil objective (Zeiss LSM 900). For confocal microscopy, z sections were collected every 3μ. Quantification of MNX1 frequency, SQSTM1 round body formation, and SOD1 aggregation was performed on confocal images by an experimenter blind to the treatment and genotype of the animals.

Statistics. All statistical analyses were performed in Prism 10 (GraphPad).

### Example 6—Phox2a Alone or in Combination with Isl1 can Reduce SQSTM1/p62 Aggregation in SOD1G93A ALS Model Mice

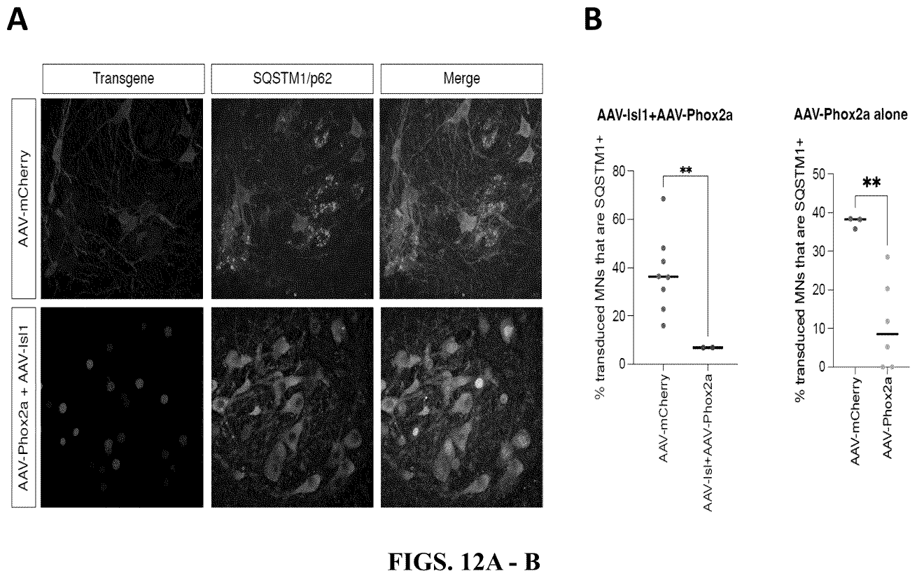

Phox2a is a transcription factor that, together with Isl1, guides the specification of cranial motor neurons during development. Cranial motor neurons are more resistant to degeneration in ALS than spinal motor neurons. SOD1G93A mice treated with AAV-ChatE-Phox2a+AAV-ChatE-Isl1, or AAV-ChatE-Phox2a alone show reduced SQSTM1 aggregation in lumbar spinal motor neurons that are vulnerable to degeneration in ALS (FIG. 12A-B).

These findings suggest that AAV-Phox2a treatment prevents protein dyshomeostasis in ALS motor neurons.

### Example 7—AAV-ChAT-E-Driven Oct4 and Sox2 can be Re-Expressed in Adult Lumbar Spinal Motor Neurons

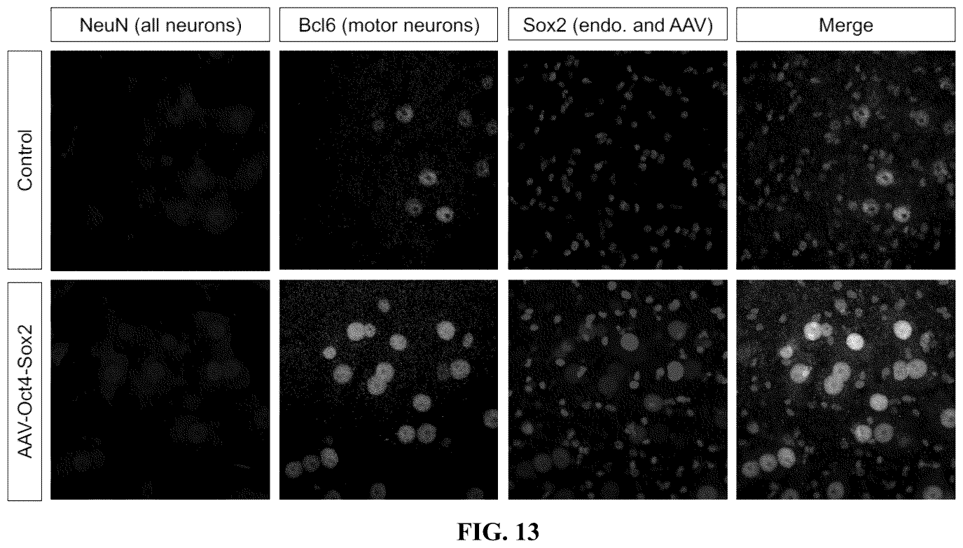

Oct4 and Sox2 are part of a suite of transcription factors that can revert terminally differentiated cells to a state of stem cell-like pluripotency. An AAV driving the expression of Oct4 and Sox2 from a bicistronic construct under the motor neuron-specific ChatE (ChAT enhancer) leads to the ectopic upregulation of these factors in adult lumbar spinal motor neurons (FIG. 13).

### Example 8—AAV-ChAT-E-Driven Oct4 and Sox2 can be Re-Expressed in Adult Lumbar Spinal Motor Neurons

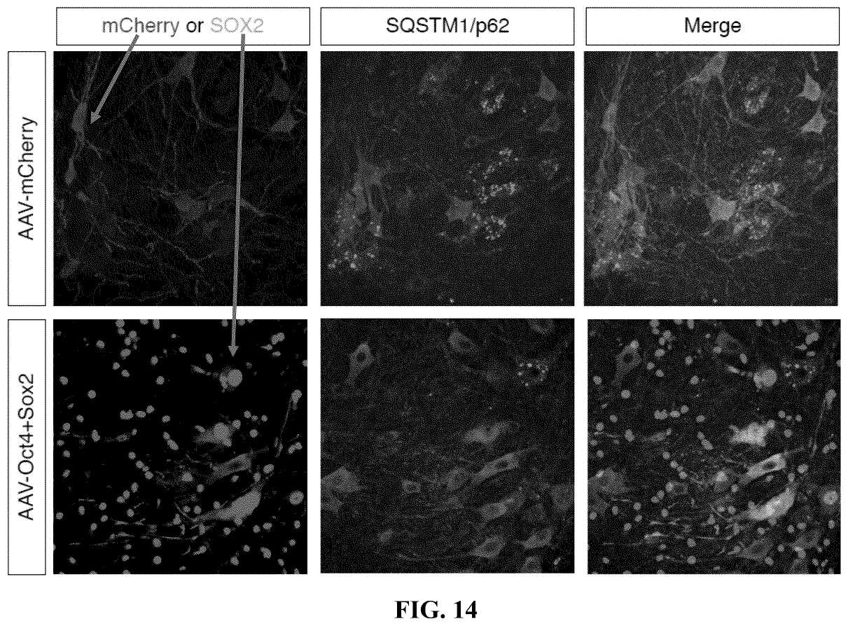

Motor neuron-specific expression of Oct4 and Sox2 appears to reduce SQSTM1 aggregation in lumbar spinal motor neurons that are vulnerable to degeneration in ALS. These findings suggest that AAV-Oct4+Sox2 treatment may prevent protein dyshomeostasis in ALS motor neurons (FIG. 14).

### REFERENCES

1. Masrori, P. & Damme, P. V. Eur. J. Neurol. 27, 1918-1929 (2020). 2. Blokhuis, A. M., Groen, E. J. N., Koppers, M., Berg, L. H. van den & Pasterkamp, R. J. Acta Neuropathol 125, 777-794 (2013). 3. Thams, S. et al. Mol Ther (2018). doi:10.1016/j.ymthe.2018.10.0104. An, D. et al. Elife8, e44423 (2019). 5. Patel, T. et al. Nat Commun 13, 5427 (2022). 6. Closser, M. et al. Neuron (2021). doi:10.1016/j.neuron.2021.10.0147. Mazzoni, E. O. et al. Nat Neurosci 16, nn.3467 (2013). 8. Rhee, H. S. et al. Neuron 92, (2016). 9. Lee, S. et al. Proc National Acad Sci 109, 3383-3388 (2012). 10. Liu, M.-L., Zang, T. & Zhang, C.-L. Cell Reports 14, 115-128 (2016). 11. Kumar, A. V., Mills, J. & Lapierre, L. R. Front. Cell Dev. Biol. 10, 793328 (2022). 12. Rudnick, N. D. et al. Proc National Acad Sci 114, E8294-E8303 (2017). 13. Gal, J., Ström, A.-L., Kilty, R., Zhang, F. & Zhu, H. J Biol Chem 282, 11068-11077 (2007). 14. Mitsui, S. et al. Mol Brain 11, 30 (2018). 15. Trist, B. G. et al. Acta Neuropathologica Commun 10, 122 (2022). 16. Fecto, F. et al. Arch. Neurol. 68, 1440-1446 (2011). 17. Watanabe, M. et al. Neurobiol. Dis. 8, 933-941 (2001). 18. Hatzipetros, T. et al. J Vis Exp (2015). doi:10.3791/5325719. Miana-Mena, F. J. et al. AmyotrophLateral Sc 6, 55-62 (2009).

### REFERENCES FOR METHODS

1. Challis, R. C. et al. Nat Protoc 14, 379-414 (2019). 2. Hatzipetros, T. et al. J Vis Exp(2015).doi:10.3791/53257

### Example 9—Embryonic Motor Neuron Programming Factors Reactivate Immature Gene Expression and Suppress ALS Pathologies in Postnatal Motor Neurons

ALS is characterized by progressive motor neuron degeneration, typically leading to paralysis and death 2-5 years after diagnosis ¹. Neurodegeneration in ALS is both cell type-specific and age-dependent, affecting only certain subtypes of motor neurons with a typical age of onset of 55-75 years. The expression of ˜7,000 genes and the accessibility of ˜100,000 chromatin regions change significantly over the course of postnatal motor neuron maturation². Considering the scale of these changes, it is challenging to pinpoint which genes or gene groups are responsible for the increased susceptibility of mature neurons to degeneration in ALS. Without being bound by theory, we reasoned that reactivation of the transcriptional master regulators that control the immature motor neuron gene expression program might be a viable strategy to prevent or delay the onset of the disease.

The majority of the genes induced in nascent motor neurons are direct targets of two transcriptional activators: ISL1 and LHX3 ³⁻⁵. ISL1 and LHX3 function as motor neuron selector transcription factors and can reprogram neural progenitors, pluripotent stem cells, and adult skin fibroblasts into immature spinal motor neurons^{4,6,7}. While essential for spinal motor neuron specification, both factors are downregulated in postnatal motor neurons (FIGS. 7A-B), and the LIM homeodomain motif that they bind becomes less accessible during postnatal motor neuron maturation². Described herein is testing of the re-expression of these factors in postnatal motor neurons in vivo to re-activate an immature gene expression state, increase the ability of motor neurons to buffer intracellular stress, and reset motor neuron resistance to the deleterious effects of ALS-causing mutations

### Results

### Chat Enhancer Yields Spinal Motor Neuron-Specific Transgene Expression

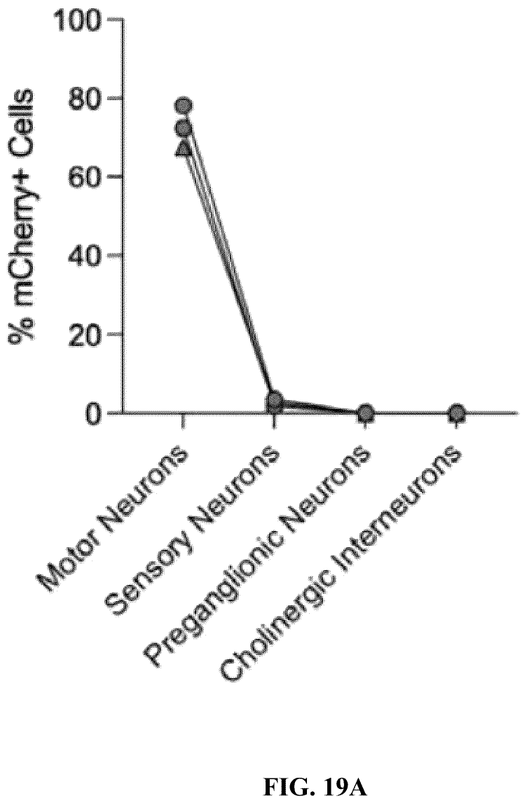

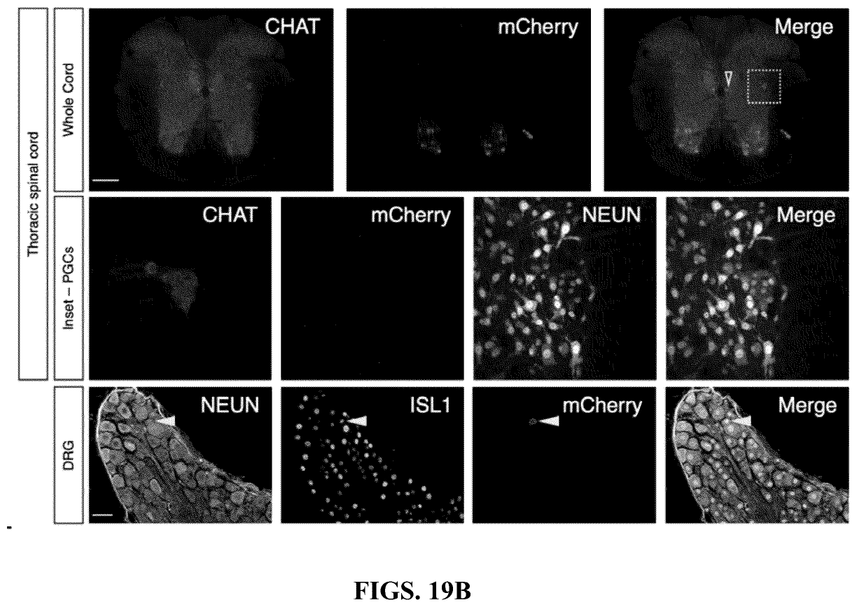

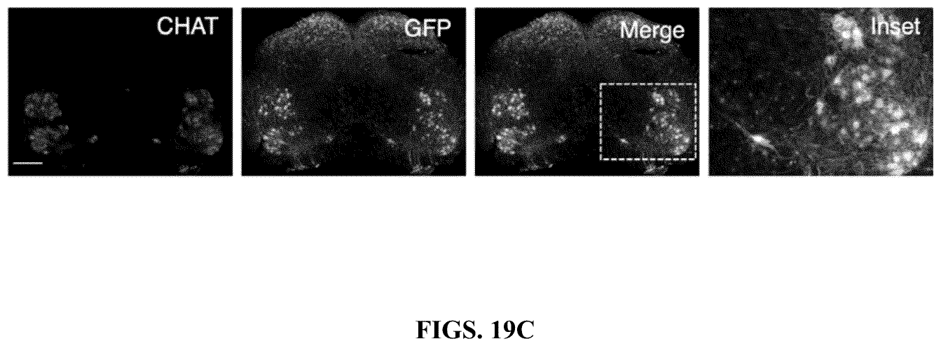

To study the effects of postnatal ISL1 and LHX3 reactivation in skeletal motor neurons, we generated adeno-associated viruses (AAVs) that drive transgene expression under the control of ChatE, a 1000 bp enhancer located 3 kb upstream of the Chat gene encoding choline acetyltransferase (FIGS. 5A-5B). We identified ChatE in a temporal ATAC-seq dataset² a chromatin region that is continuously accessible in spinal motor neurons throughout postnatal life (FIG. 5A) and contains binding sites for a suite of transcription factors that control motor neuron specification and maturation (FIG. 7C) ^{2,4,5}. To evaluate the efficiency and specificity of ChatE-driven expression, AAV-mCherry was administered by intracerebroventricular injection (3.19E⁺10 vg/animal) in neonatal mice at postnatal day 1 (P1; FIG. 19A-B). Strong, motor neuron-specific expression of mCherry was observed by immunohistochemistry (IHC) in the lumbar spinal cord within 7 days post-injection. Other local CHAT-expressing cells, such as V0c interneurons and preganglionic cells, remained negative for mCherry, as did sensory neurons in dorsal root ganglia (FIGS. 19A-B). In contrast, PHP.eB AAVs generated from a second reporter construct where NLS-GFP was driven by a ubiquitous CAGGS promoter and administered under similar conditions (2.10E+10 vg/animal at P1) yielded widespread GFP expression throughout the spinal cord in both CHAT⁺ and CHAT⁻ cells (FIG. 19C).

### Viral Reexpression of ISL1 and LHX3 in Motor Neurons

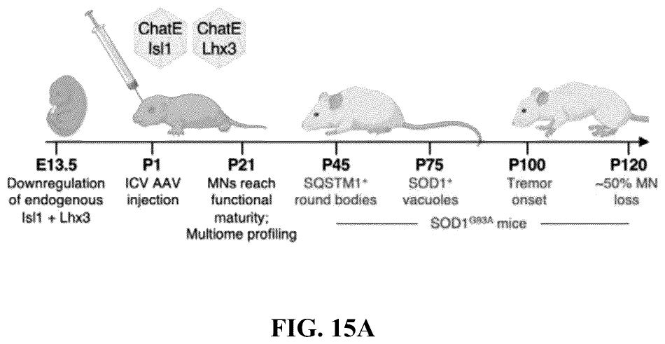

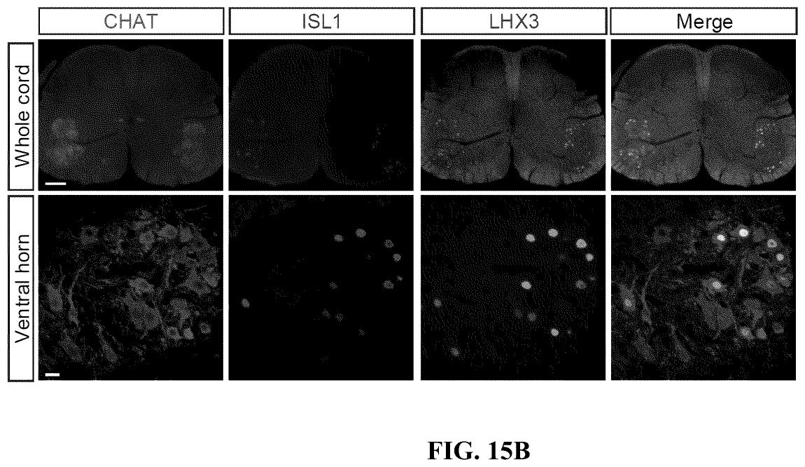

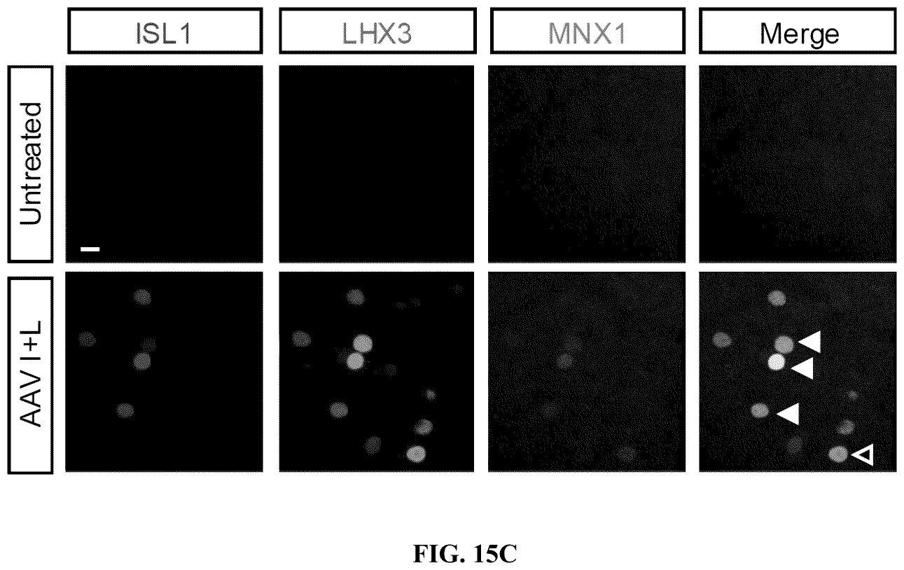

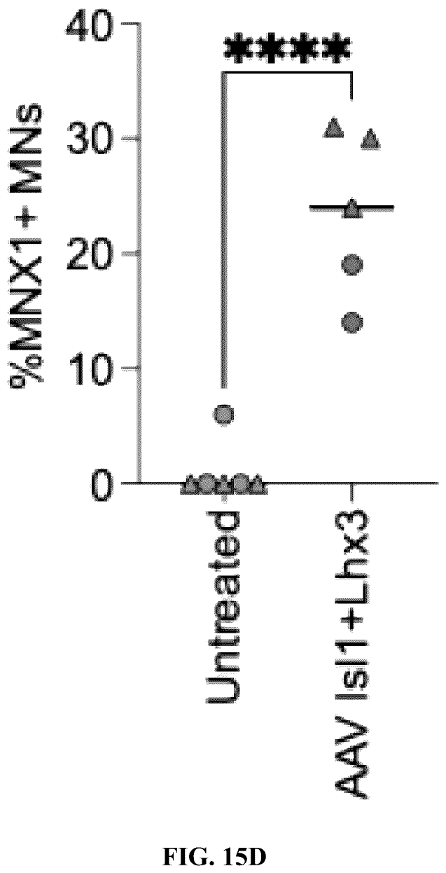

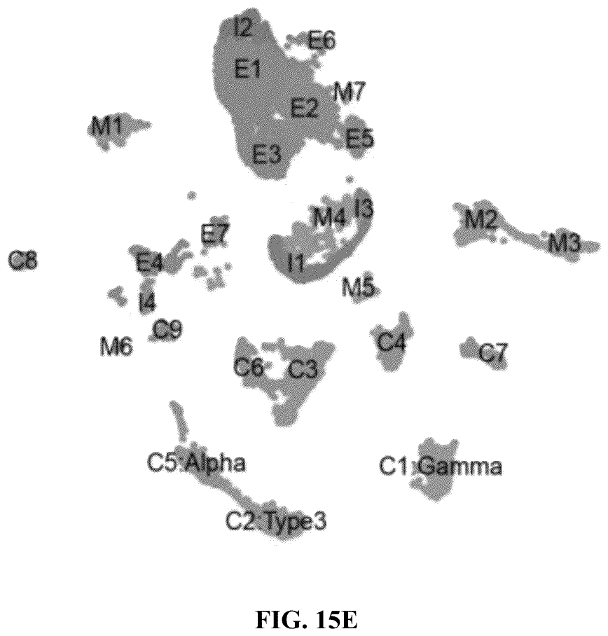

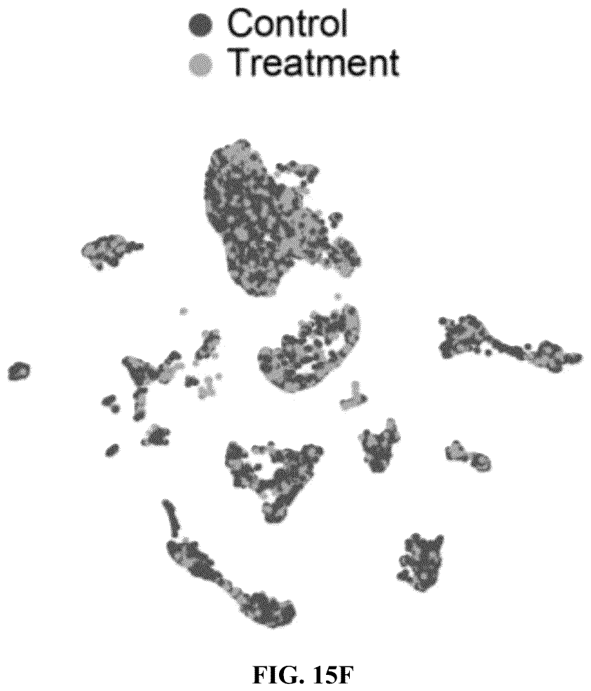

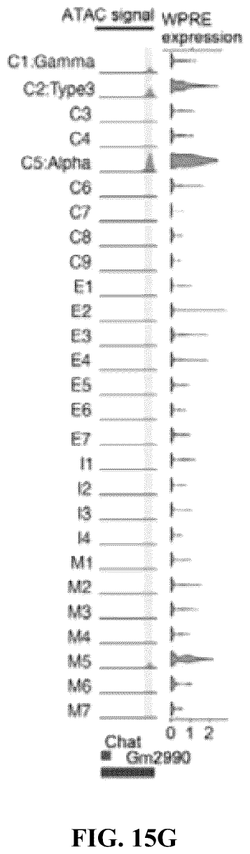

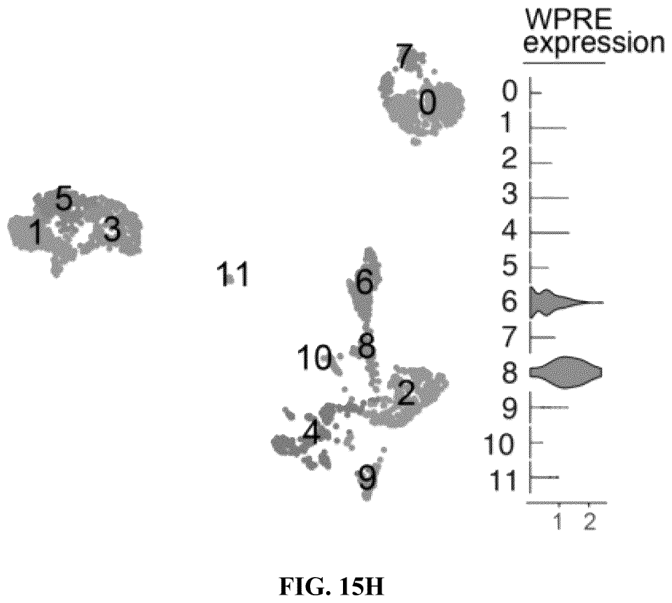

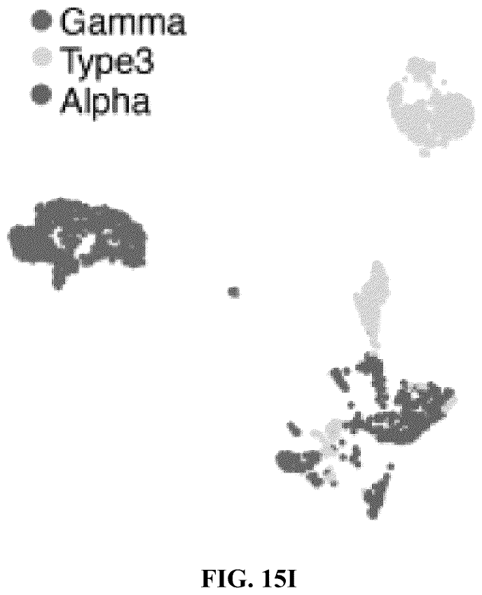

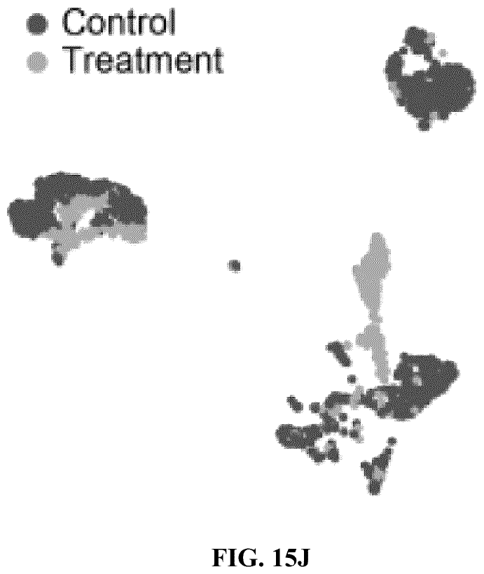

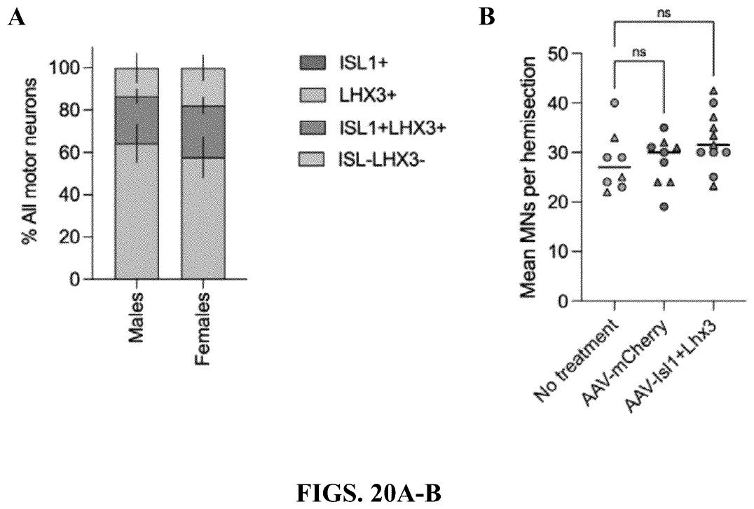

We then evaluated the distribution and transduction efficiency of AAV-Isl1 and AAV-Lhx3 administered at a 1:1 ratio across a range of viral titers (FIG. 15A). Under both low titer (6-9E⁺10 vg/animal) and high titer (3-4E⁺11 vg/animal) transduction conditions, we observed that ˜90% of CHAT⁺ motor neurons in the lumbar spinal cord were positive for transgene expression at early post-injection time points (7-14 days), with near-complete overlap between ISL1⁺ and LHX3⁺ cells (FIG. 15B). Under low titer conditions, the percentage of transgene-expressing motor neurons dropped to ˜22% by P45, with 11% co-expressing ISL1 and LHX3, 2% expressing ISL1 alone, and 9% expressing LHX3 alone (FIG. 5D). In animals treated with AAV-mCherry under similar low titer conditions, the percentage of mCherry⁺ motor neurons remained similar across all time points assessed (˜70-80%; see FIGS. 19A-B), suggesting that the downregulation of ISL1 and LHX3 was selective. Under high titer transduction conditions, LHX3 expression appeared to stabilize, while ISL1 continued to decrease with time (FIG. 7D). By P45, 84% of motor neurons retained expression of AAV-driven LHX3, with 23% co-expressing ISL1 and LHX3 and 61% expressing LHX3 alone (FIG. 5D). Under both low and high titer conditions, AAV-Isl1+AAV-Lhx3 treatment resulted in similar transduction efficiencies between sexes (FIG. 20A) and appeared to be well-tolerated, with no impact on basal motor neuron survival relative to AAV-mCherry-treated or untreated controls (FIG. 20B).

### Global Gene Expression and Chromatin Accessibility Profiling of Transduced Neurons

To evaluate whether heterochronically re-expressed ISL1 and LHX3 could reactivate their embryonic targets, we stained lumbar spinal cord tissue collected at P45 for MNX1 (HB9), a gene that is specifically expressed in nascent spinal motor neurons and is downregulated before birth (FIG. 7E). We found that MNX1 was virtually undetectable in untreated or control AAV-treated animals (FIGS. 15C-D). Treatment with AAV-Isl1+AAV-Lhx3 led to significant upregulation of MNX1 (p<0.0001; FIGS. 15C-D), with 50% AAV-Isl1⁺ motor neurons and 24% of motor neurons overall exhibiting MNX1 expression. We also observed a subset of MNX1⁺ motor neurons that were ISL1⁻LHX3⁺ at this time point (FIG. 15C), which may reflect the rapid downregulation of ISL1 and the prevalence of ISL1⁻LHX3⁺ motor neurons overall (FIG. 5D).

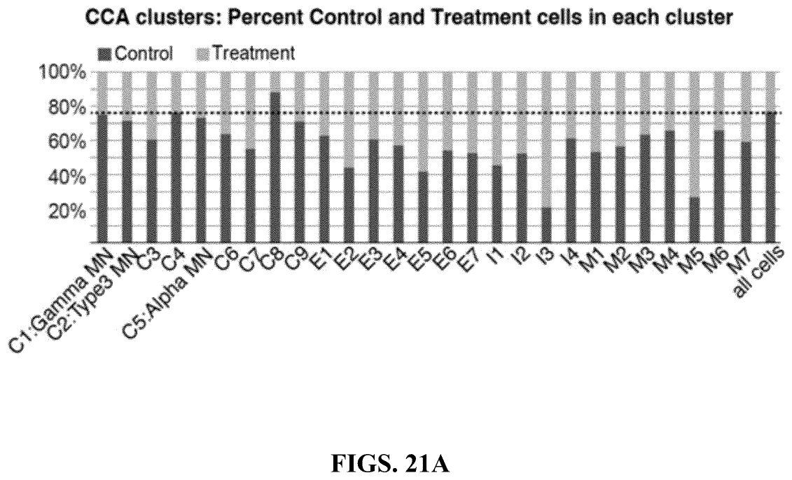

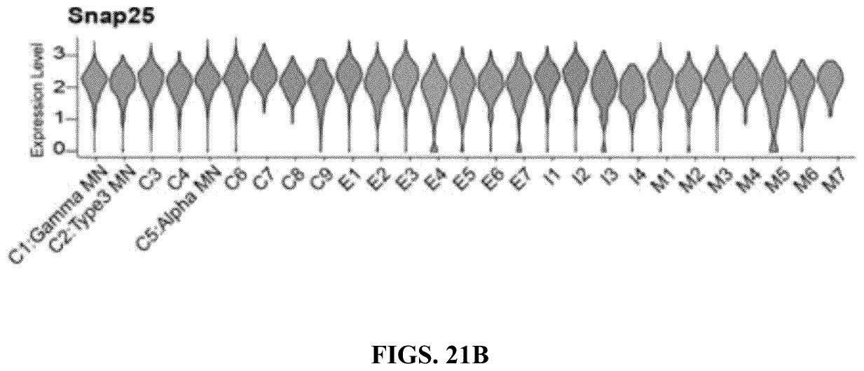

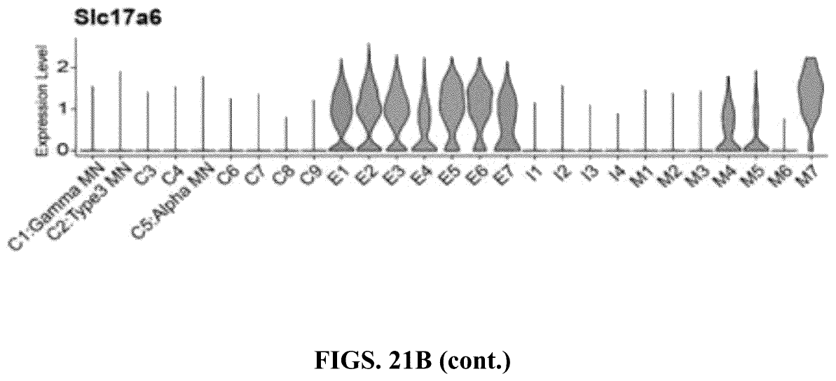

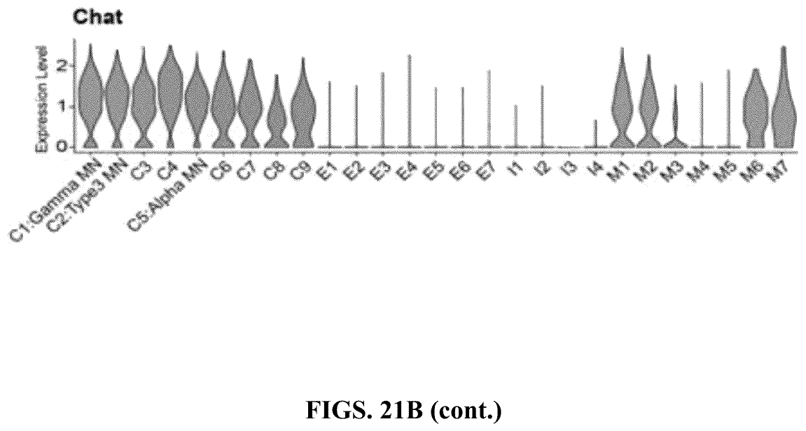

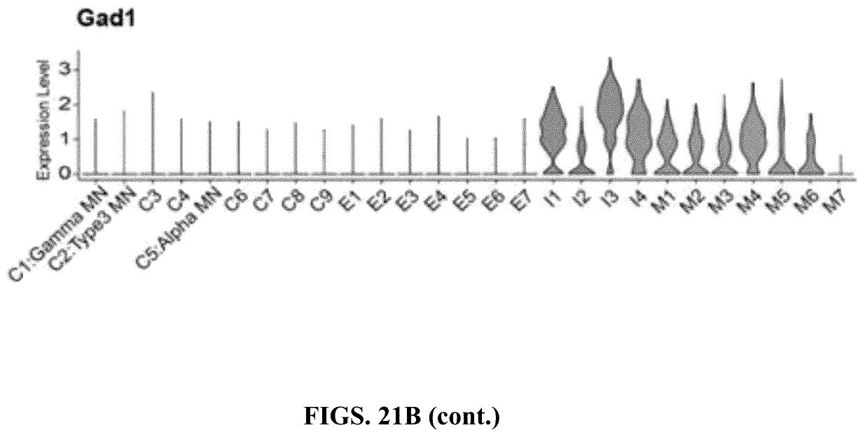

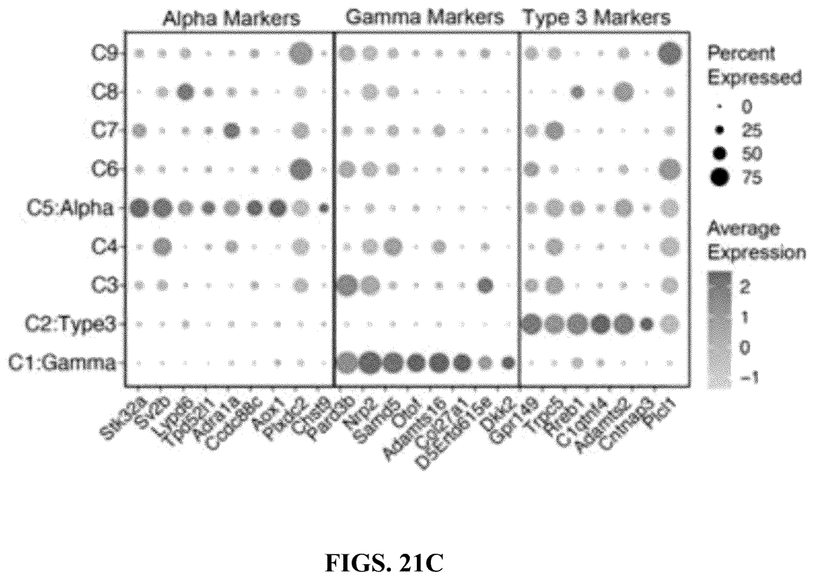

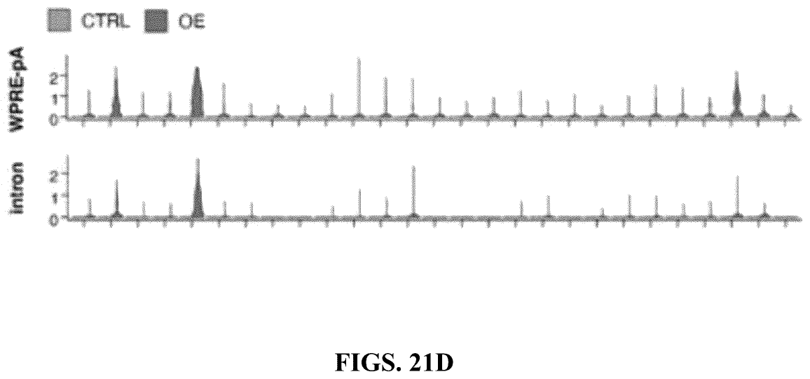

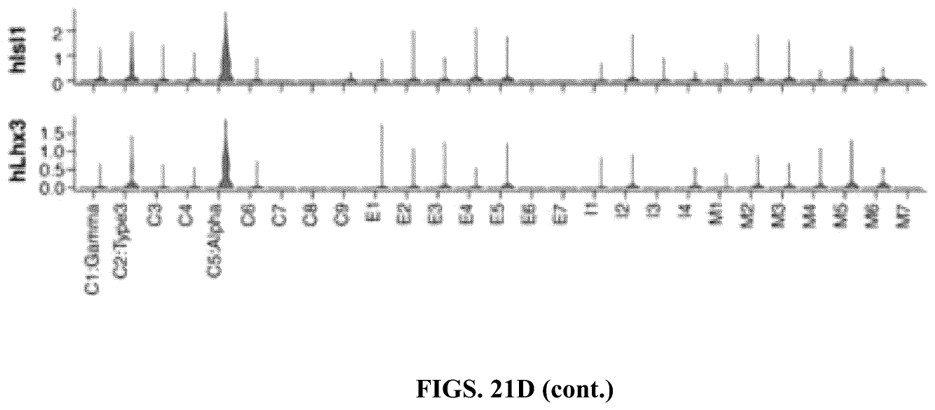

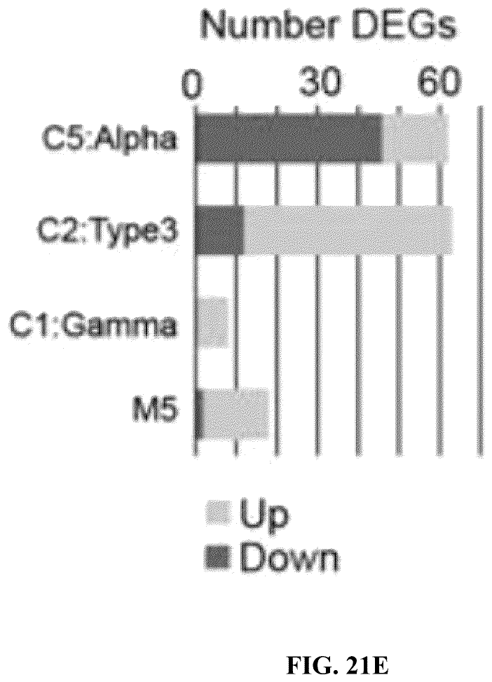

The reactivation of MNX1 prompted us to evaluate the global effects of ISL1 and LHX3 re-expression on postnatal motor neuron gene expression and chromatin accessibility by performing single nucleus multiome RNA and ATAC sequencing in the spinal cord. To enrich for motor neuron nuclei in whole spinal cord samples, we used Chat-Cre;Sun1-sfGFP-Myc (INTACT) mice that express a nuclear envelope-bound GFP reporter driven by Chat ^{2,8}. INTACT mice were either injected at P1 with AAV-Isl1+AAV-Lhx3 under high titer conditions (3E⁺11 vg/animal; Treatment group; n=8 mice) or left untreated (Control group; n=6 mice). GFP⁺ nuclei from both conditions were then isolated and sequenced at P21. RNAseq reads were aligned to a custom genome that included four additional elements present in the AAV constructs to identify AAV⁺ nuclei within the treatment group: the human ISL1 and LHX3 transgene-coding sequences, the chimeric intron, and the WPRE 3′UTR element (FIG. 5B). Canonical correlation analysis (CCA)-based integration of the two datasets resulted in the identification of 27 clusters (FIG. 15E) populated by nuclei from both control and experimental animals (FIGS. 15F, 21A-B). Using motor neuron markers defined by previous single nuclei RNAseq studies ^{8,9}, we identified three clusters corresponding to alpha motor neurons (C5), gamma motor neurons (C1), and type 3 motor neurons (C2) (FIGS. 15E and 21C). Consistent with the motor neuron specificity of ChatE, expression of AAV transgenes was largely restricted to the motor neuron clusters (FIGS. 15G and 21D): 90.5% of alpha motor neuron nuclei, 70.9% of type 3 motor neuron nuclei, and 26.5% of gamma motor neuron nuclei from the treatment group expressed at least one of the four AAV elements. We identified one additional non-motor neuron cluster (M5) with 55% of nuclei expressing AAV transcripts. ATACseq analysis confirmed that the three motor neuron clusters, as well as the M5 cluster, exhibited increased chromatin accessibility at the ChatE locus, compared to clusters that did not express ChatE-driven transgenes (FIG. 15G).

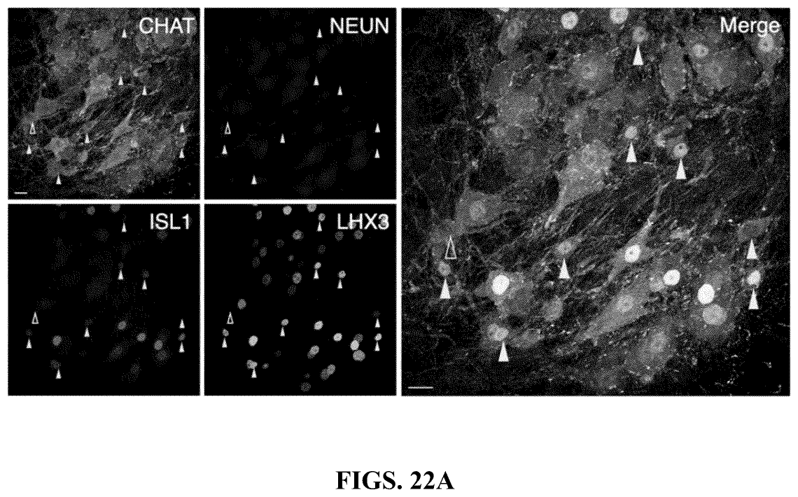

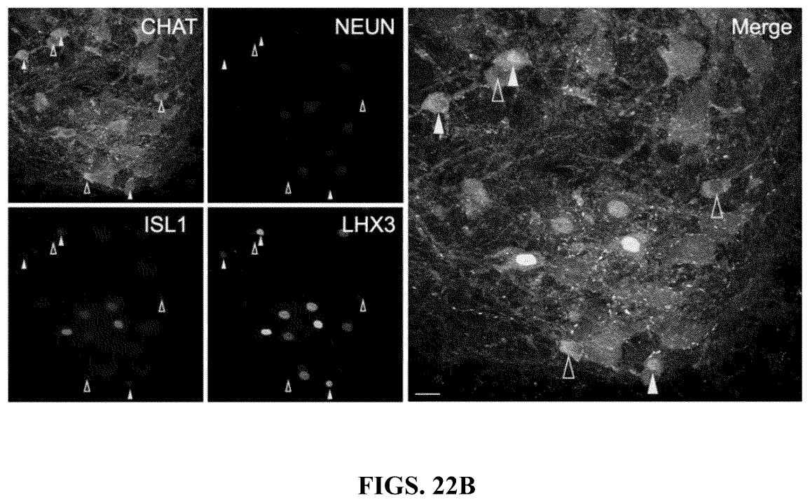

To understand the impact of treatment on gene expression, we performed differential gene expression analysis for each of the four AAV-expressing clusters. Within each cluster, all AAV⁺ nuclei were compared with an equal number of randomly selected nuclei from the control group. This analysis showed that >60 genes were differentially expressed in both alpha and type 3 clusters, while fewer than 20 genes were differentially expressed in the gamma or M5 clusters (FIG. 21E). The relative dearth of gene expression changes in gamma motor neurons was especially surprising, given that ISL1 and LHX3 proteins can be detected in these cells by IHC (FIGS. 22A-B). Together, these data suggest that the effects of heterochronically re-expressed ISL1 and LHX3 are not only specific to motor neurons over other cell types but are also selective among motor neuron subtypes.

### Motor Neuron Subtype-Specific Effects of ISL1 and LHX3 on Gene Expression

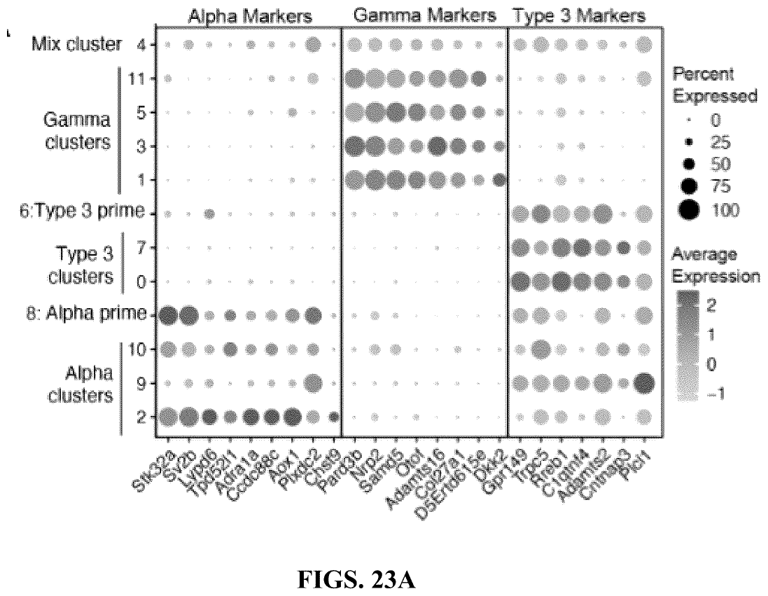

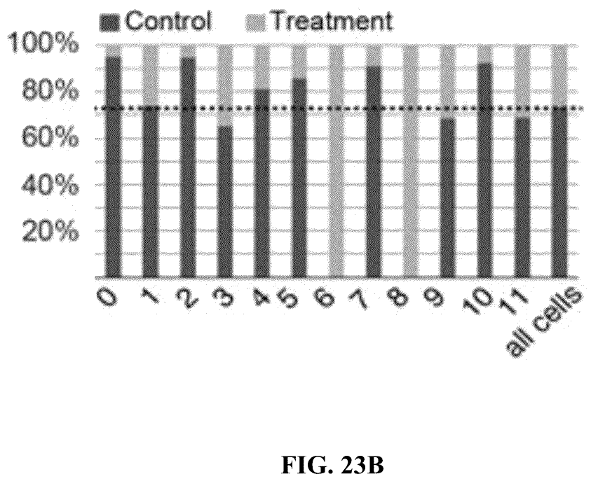

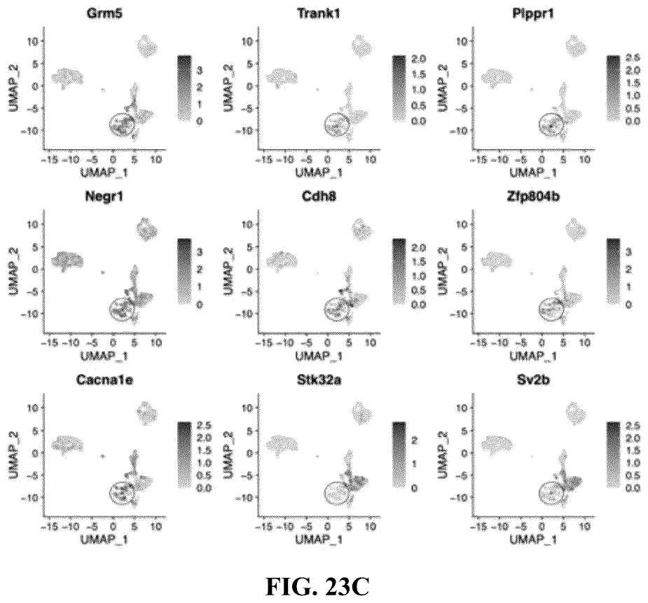

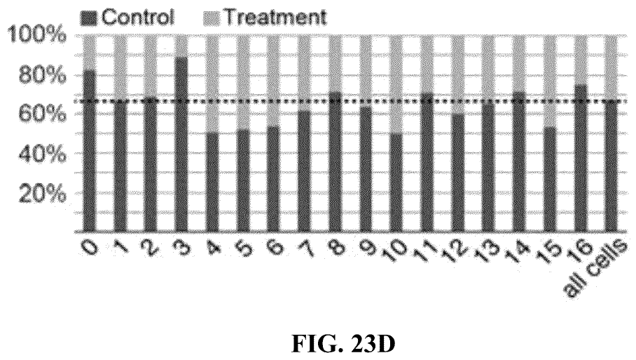

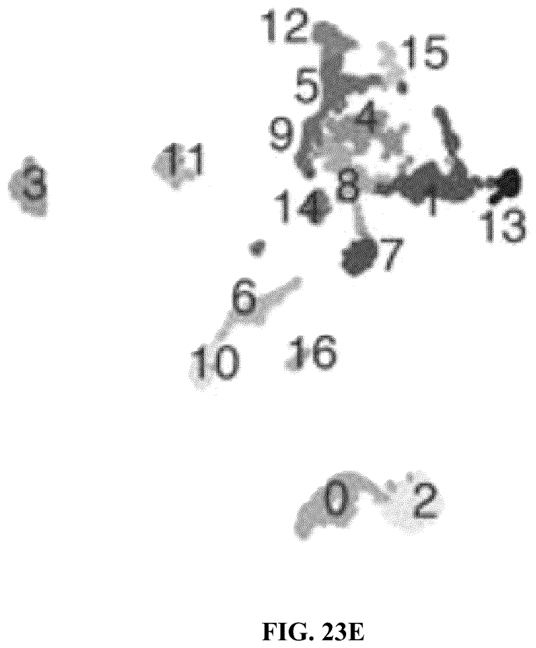

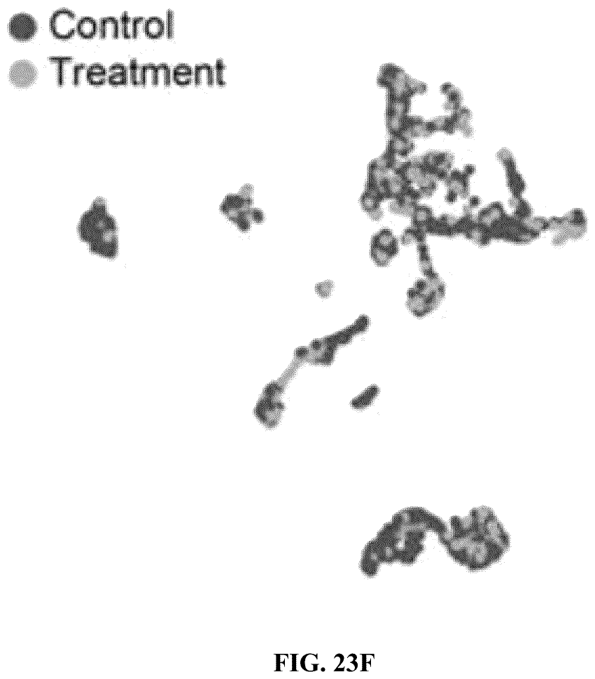

Considering the impact of AAV-Isl1+AAV-Lhx3 on gene expression on alpha and type 3 motor neurons, we asked whether selectively re-clustering motor neuron nuclei without CCA-based integration would result in the segregation of control vs. treatment nuclei (FIGS. 15H-J). Indeed, we found that most type 3 and alpha motor neuron nuclei in the treatment group formed unique clusters (6 and 8, respectively) that separated from the type 3 (0 and 7) and alpha (2, 9, and 10) motor neuron clusters populated primarily by control nuclei (FIGS. 15I, J, 23A-B). We termed these unique clusters alpha prime and type 3 prime. In contrast, gamma motor neuron nuclei from both conditions populated the same clusters (FIGS. 15H, J), consistent with the minimal gene expression changes observed in these cells (FIG. 21E). We also identified a cluster(4) that appeared to contain a mixture of cell types from the original clustering (FIGS. 15H-J). Based on the top markers in this cluster (Grm5⁺, Trank1⁺, Plppr1⁺) it seemed to correspond a similar cluster in published adult motor neuron single cell data that is found near alpha neurons, but lacks expression of strong alpha markers (Stk32a⁻, Sv2b⁻) (FIG. 23C). To ensure that the separation of alpha prime and type 3 prime clusters from their respective control clusters was due to biological rather than technical reasons, we used the same methods to re-cluster the non-motor neuron cholinergic nuclei and found that all resulting clusters had similar proportions of control and treatment nuclei (FIGS. 23D-F).

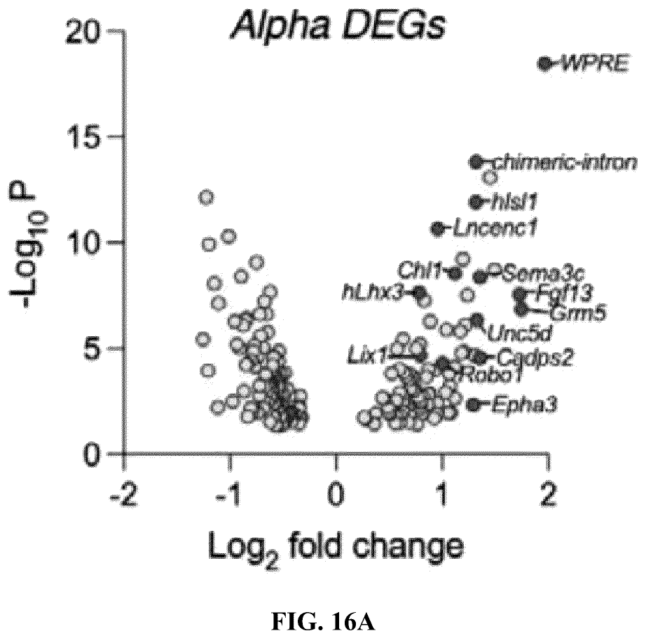

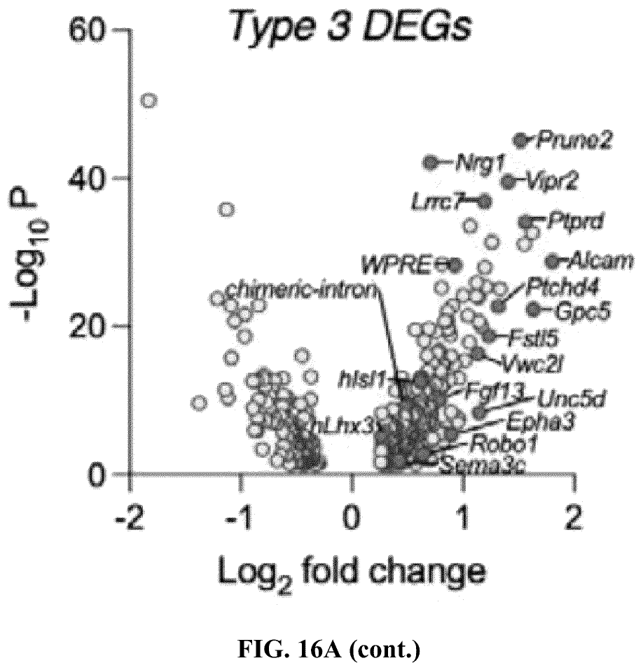

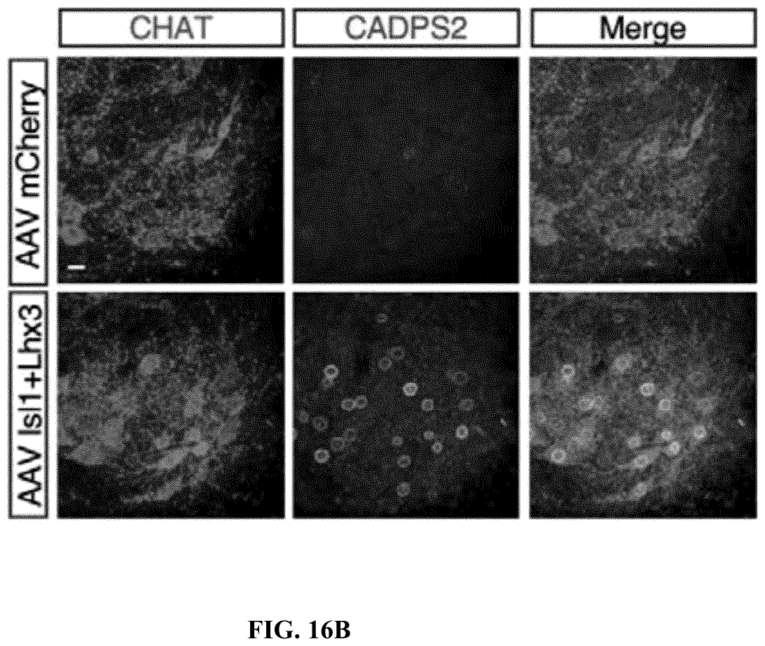

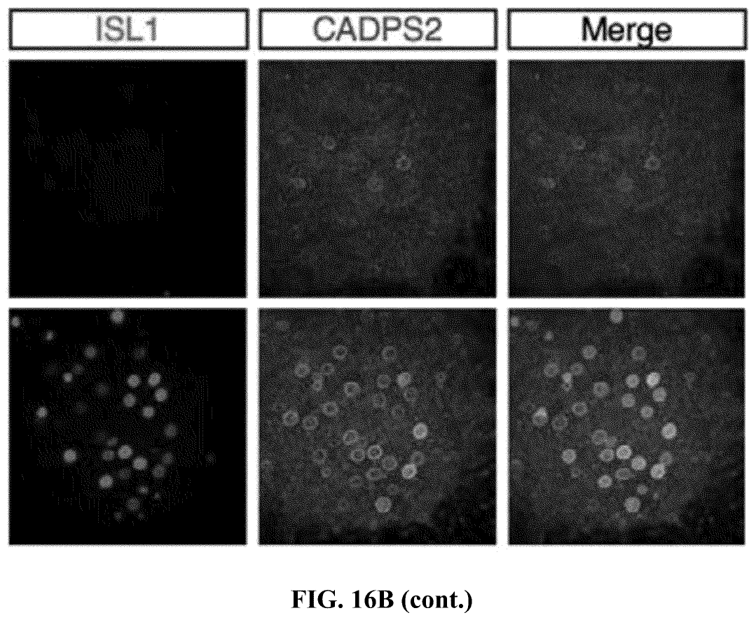

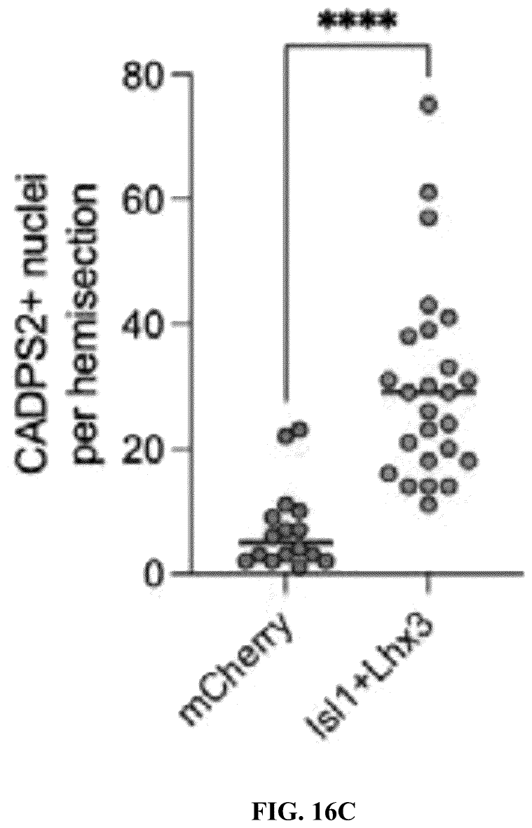

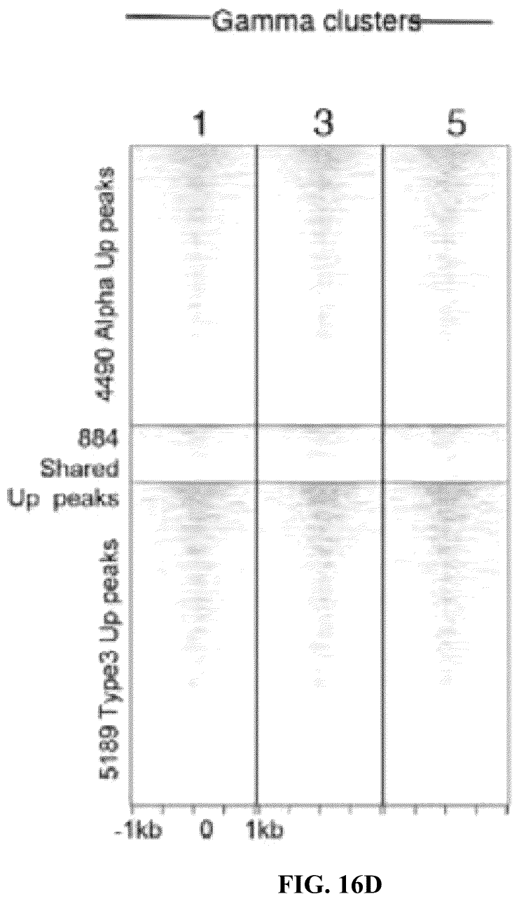

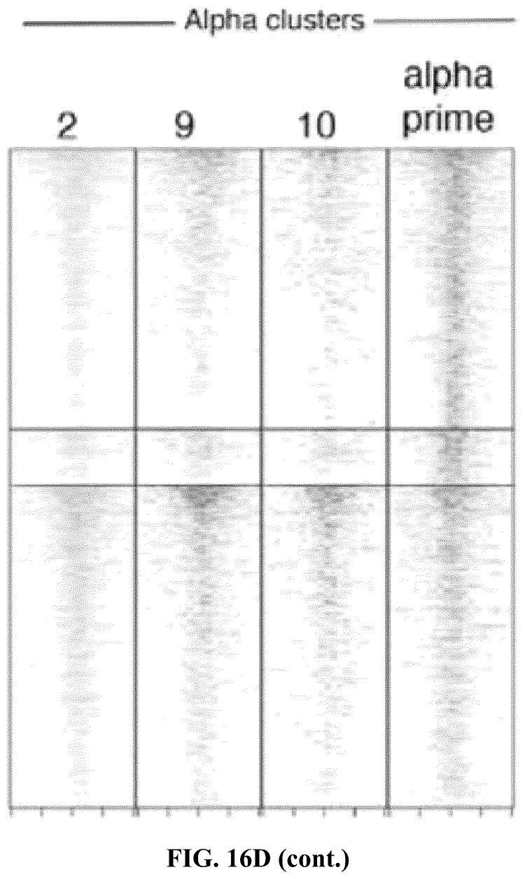

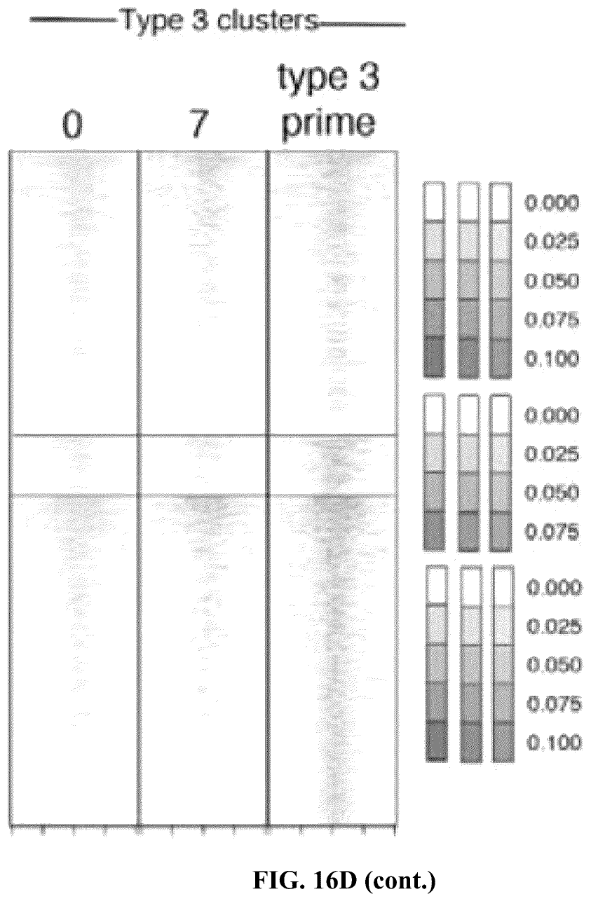

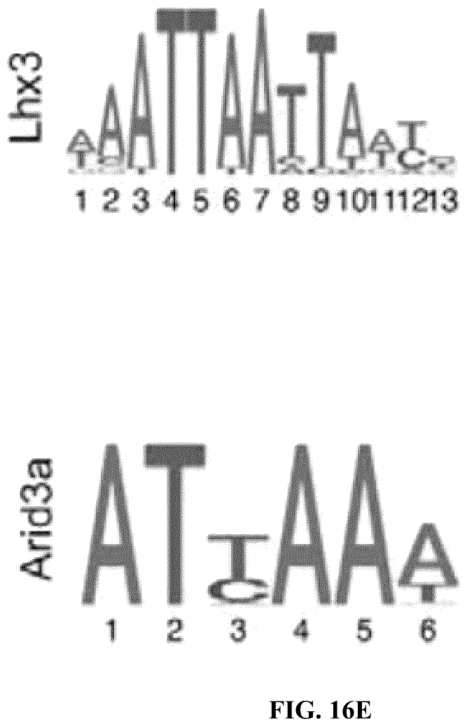

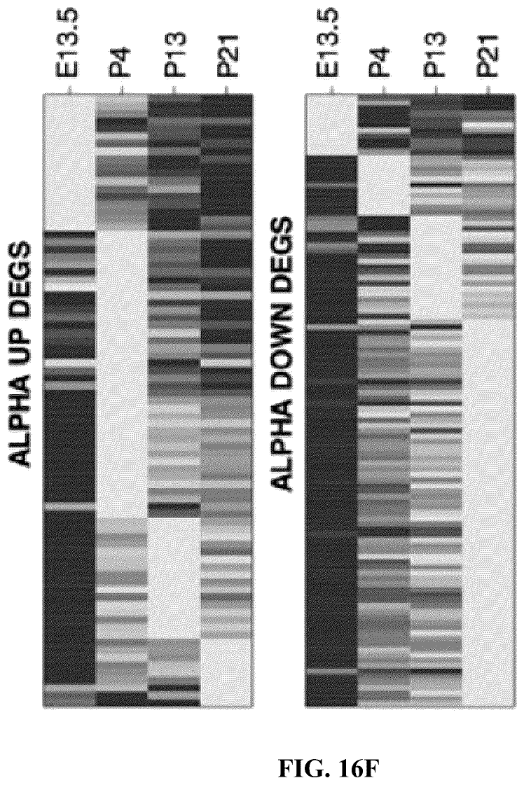

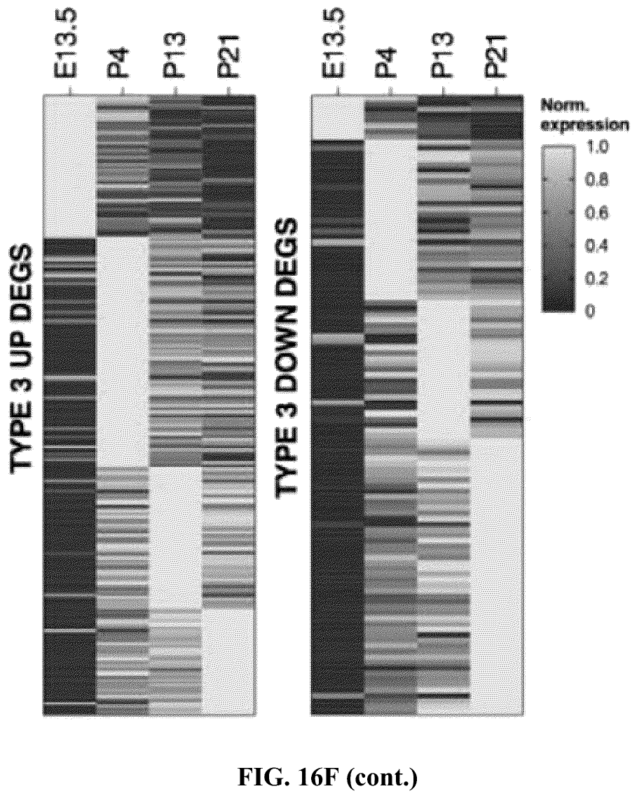

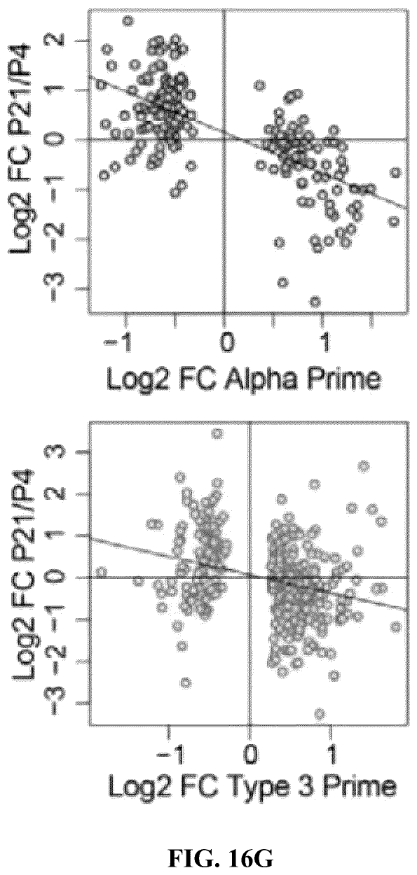

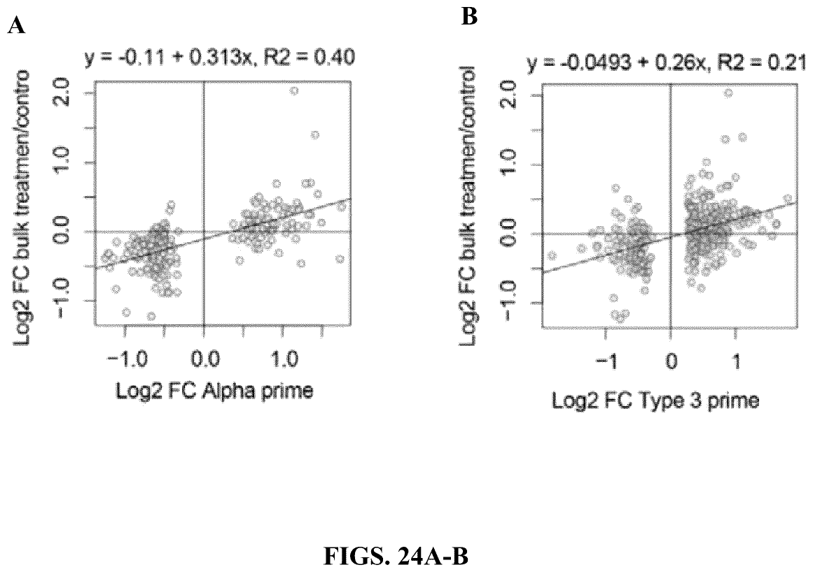

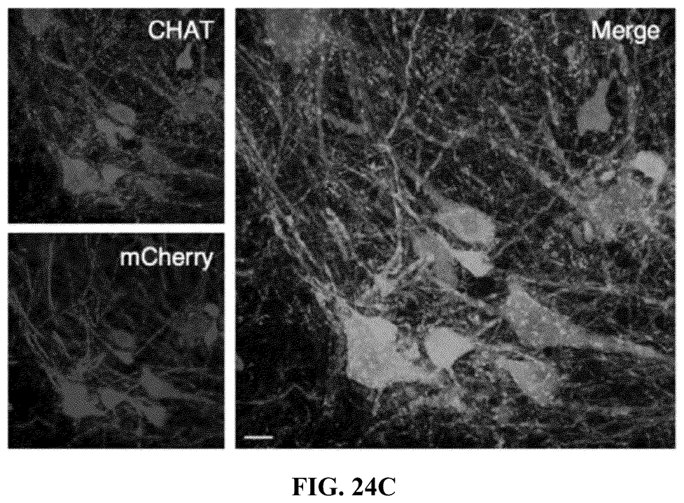

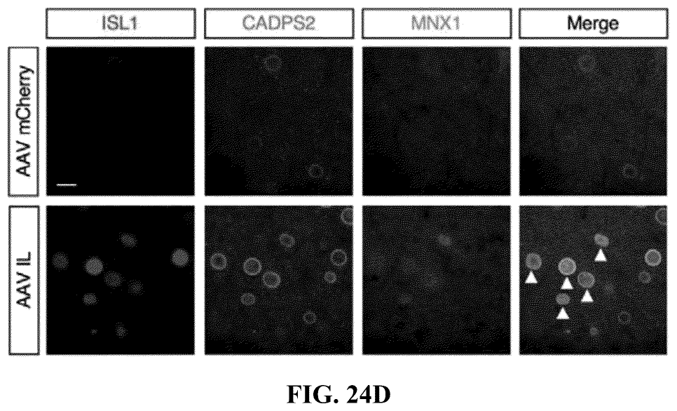

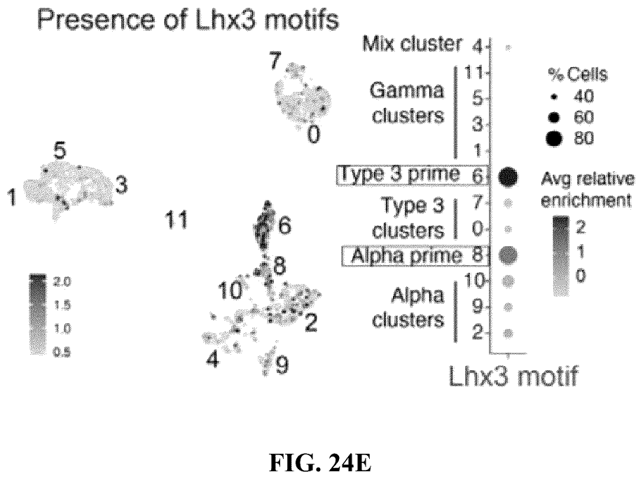

The formation of treatment-specific clusters allowed us to focus our subsequent analyses on the cells most impacted by ISL1 and LHX3 re-expression. Differential gene expression analysis identified 85 genes significantly upregulated and 112 genes significantly downregulated in alpha prime vs. the remaining alpha nuclei (referred to hereafter as alpha differentially-expressed genes (DEGS)), and 244 genes significantly upregulated and 112 genes significantly downregulated in type 3 prime vs the remaining type 3 nuclei (type 3 DEGs; FIG. 5A). Only 83 genes, or 19% of total DEGs, were shared between the alpha and type 3 motor neurons, indicating that the effects of transgene expression were largely subtype-specific even among the cell populations that are strongly AAV-positive. To independently validate the single nuclei differential gene expression, we performed bulk RNA-seq analysis of GFP⁺ cells isolated from P21 INTACT control mice and mice treated with AAV-Isl1+AAV-Lhx3 or AAV-mCherry at P1. Despite the lower sensitivity of the bulk sequencing dataset, we found a significant positive correlation between the expression of both the alpha and type 3 DEGs in the single nuclei and bulk sequencing datasets (alpha DEG R²=0.40, p<0.0001; type 3 DEG R²=0.21, p<0.0001; FIGS. 24A-B). Finally, we used IHC to confirm the differential expression of CADPS2, one of the top induced genes in alpha prime neurons. Staining of lumbar spinal cord sections from an independent set of control and high titer AAV-Isl1+AAV-Lhx3 treated mice revealed a significant upregulation and striking perinuclear accumulation of CADPS2 protein in transgene-expressing motor neurons (FIGS. 16B-C, 24C-D).

Putative Regulatory Elements that Gain Accessibility are Enriched for LHX3-HD Binding Motif

Cooperative binding of ISL1 and LHX3 to DNA in differentiating embryonic stem cells results in a transient increase in chromatin accessibility that is rapidly lost following Lhx3 downregulation in maturing motor neurons ⁵. To investigate whether re-expression of these factors in postnatal neurons changed chromatin accessibility at P21, we examined the single nucleus ATACseq dataset. We first performed differential peak analysis, which led to the identification of 5374 upregulated and 2981 downregulated peaks (among ˜89,000 total accessible sites) in alpha prime compared to alpha nuclei, and 6073 upregulated and 2644 downregulated peaks (among ˜87,000 total accessible sites) in type 3 prime compared to type 3 nuclei (FIG. 16D, upregulated peaks shown). Next, we asked which transcription factor motifs were enriched in the regions that gained or lost chromatin accessibility following ISL1 and LHX3 re-expression. For both alpha prime and type 3 prime motor neurons, the top motifs enriched in upregulated peaks were Lhx3 and Lhx3-like LIM homeodomain motifs (FIG. 16E), though there was only minimal overlap (˜8%) of the chromatin domains that gained accessibility between each cluster, echoing the motor neuron subtype-specificity of the DEGs. Lhx3 motifs were not enriched among upregulated peaks in gamma motor neurons (FIG. 16E), nor among downregulated peaks in any motor neuron subtype. Together these findings suggest that re-expressed ISL1 and LHX3 increase chromatin accessibility at genomic loci that harbor canonical Lhx3 binding motifs, which parallels their activity during normal motor neuron development ⁴, but that their activity in postnatal motor neurons is subtype-specific.

To further explore the relationship between changes in chromatin accessibility with changes in gene expression, we asked how many of the alpha and type 3 DEGs were the closest genes to the differential peaks found in each motor neuron subtype. We found that 70% of upregulated alpha DEGs and 87% of upregulated type 3 DEGs were the closest gene to at least one chromatin region that gained accessibility, compared to only 32% and 22% of downregulated alpha and type 3 DEGs. Together, the observations that Lhx3 motifs are preferentially enriched in upregulated peaks, and that upregulated peaks are preferentially associated with upregulated genes, suggest that ISL1 and LHX3 act as transcriptional activators when they are re-expressed postnatally, much like they do during development^{3,5,6}.

### Activation of Early Postnatal Gene Expression Program in Transduced Motor Neurons

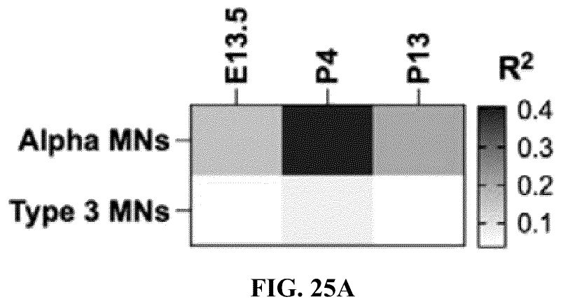

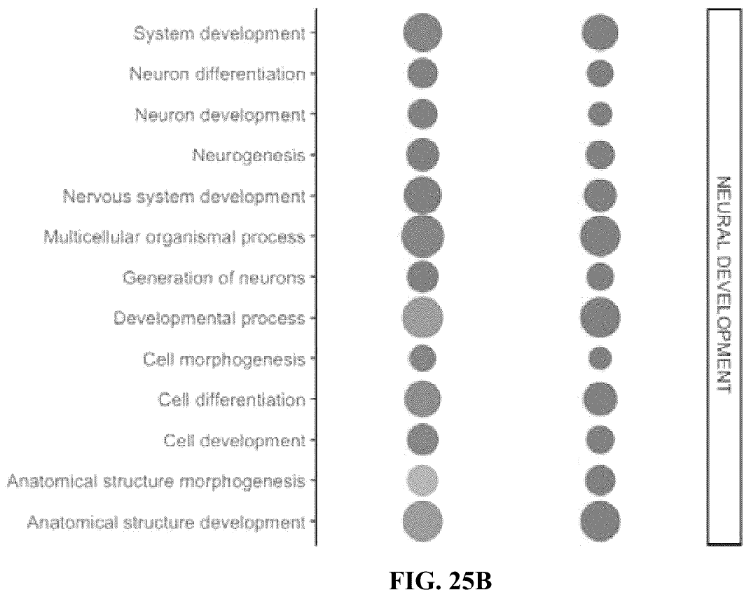

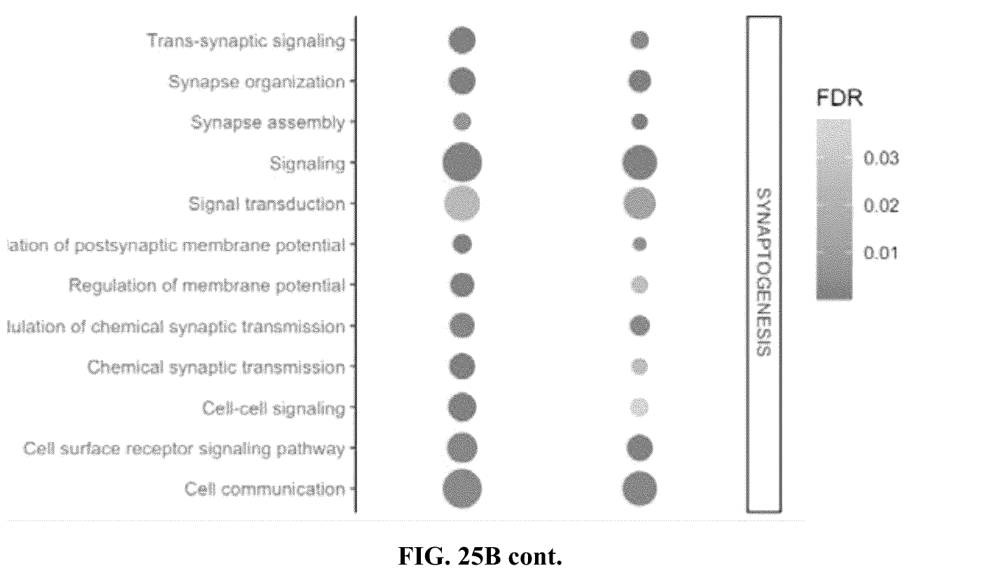

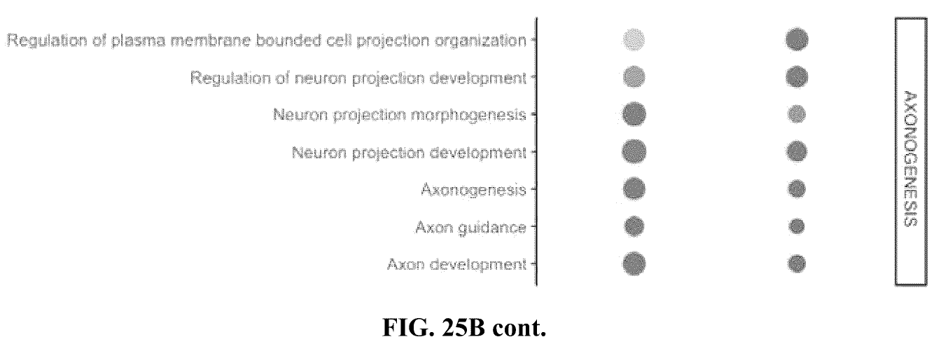

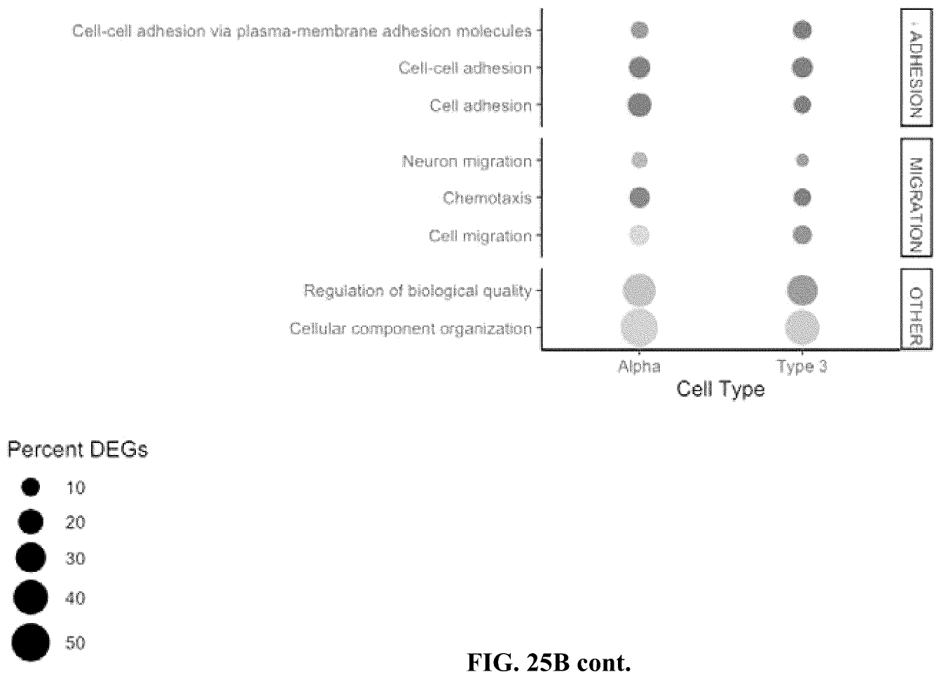

Encouraged by the major changes in gene expression and accessibility observed in AAV-transduced cells, we set out to evaluate whether heterochronic ISL1 and LHX3 re-expression was preferentially activating genes expressed in immature motor neurons. As a benchmark for transcriptional “age” of the alpha and type 3 prime nuclei, which were collected at P21 from animals treated at P1, we used longitudinal motor neuron transcription profiles generated by bulk sequencing of purified INTACT nuclei at E13.5, P4, P13, and P21 ². We first examined the normal temporal expression patterns of all alpha and type 3 DEGs. Among both motor neuron subtypes, we found that most of the genes that were upregulated in response to AAV-Isl1+AAV-Lhx3 treatment normally peak during embryonic and early postnatal ages (E13.5 or P4; FIG. 16F). Conversely, genes that were downregulated in response to AAV-Isl1+AAV-Lhx3 treatment are normally most highly expressed as motor neurons approach functional maturity (P21; FIG. 16F). We then compared AAV-driven changes in the alpha and type 3 DEGs vs. maturation-driven changes in gene expression between P21 and E13.5, P4, or P13. We found significant negative correlations across all comparisons, which were strongest among genes that are differentially expressed between P21 and P4 (FIGS. 16G, 25A). Together, these data suggest that re-expressed ISL1 and LHX3 activate a gene expression subprogram that is characteristic of perinatal spinal motor neurons.

This idea was further underscored by gene ontology (GO) analyses of the upregulated alpha and type 3 DEGs, where the most significantly enriched terms for both cell types fell into categories relating to neural development, synaptogenesis, and axonogenesis (FIG. 25B). Indeed, many of the shared, upregulated alpha and type 3 DEGs have well-established roles in motor neuron development, including Sema3c¹⁰, Epha3^{11,12} and Robo1¹³. Thus, while ISL1 and LHX3 re-expression yielded a host of cell type-specific DEGs, it appeared to have the same global effect of promoting a less mature state in both alpha and type 3 motor neurons.

### ISL1 and LHX3 Re-Expression Prevent the Formation of SQSTM1-Positive Round Bodies in a Mouse Model of ALS

To probe the impact of reactivating perinatal motor neuron genes on late-onset motor neuron degeneration, we evaluated AAV-Isl1 and AAV-Lhx3 in the SOD1^{G93A} transgenic mouse model of ALS, where most motor neuron pathologies occur in mature cells. This mouse model recapitulates many biochemical and behavioral features of the disease, including severe deficits in the processing of misfolded proteins in vulnerable spinal motor neuron populations, followed by neuromuscular junction denervation, motor neuron degeneration, neuroinflammation, and clinical symptoms that progress swiftly until endstage at ˜P160.

One of the earliest histological markers of disease in this model is dysregulation of SQSTM1, a key component of both the ubiquitin-proteasome system (UPS) and the macroautophagy pathway that regulates lysosomal protein degradation¹⁴. Large, round aggregates of SQSTM1 (termed “round bodies” ¹⁵) are detectable in the cytoplasm of lumbar motor neurons by P35 ¹⁶, and by P45, they can be found in nearly one-third of motor neurons (see FIG. 6A). Highlighting the relationship between proteostatic defects and clinical severity, motor neuron-specific knockout of the autophagy gene Atg7 in SOD1^{G93A} mice enhances SQSTM1 aggregation and hastens the onset of clinical symptoms¹⁵. Overexpression of SQSTM1 itself promotes the cytoplasmic aggregation of mutant SOD1 and accelerates the onset of clinical phenotypes in SOD1^{H46R} mice ¹⁷. Importantly, SQSTM1-positive aggregates that are co-positive for TDP43 and other ubiquitinated proteins are commonly observed in postmortem spinal cord tissues from both familial and sporadic ALS patients¹⁸, and mutations in SQSTM1 have been causally linked to ALS¹⁹, reinforcing the translational relevance of SQSTM1 dysregulation.

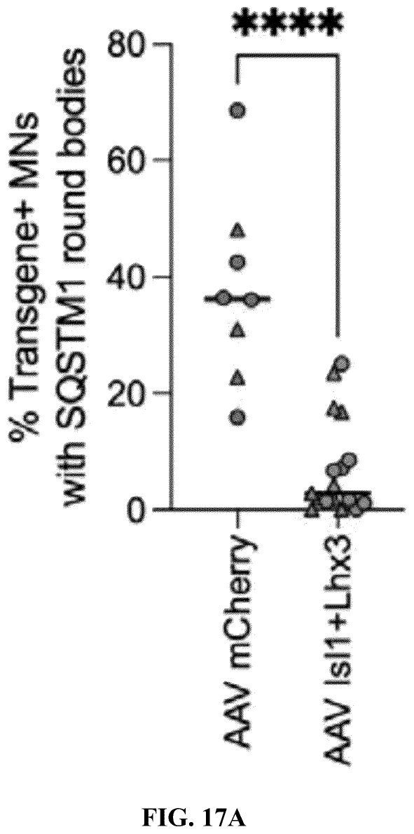

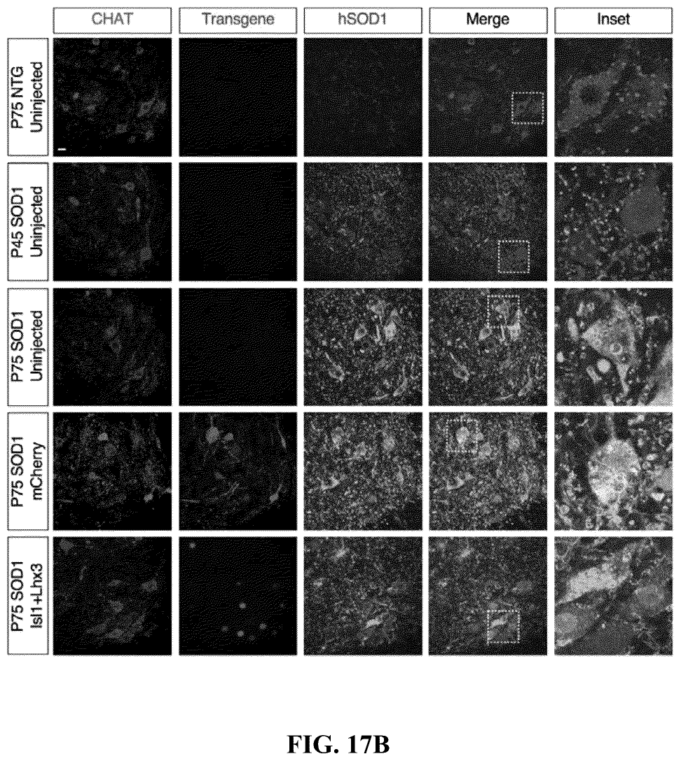

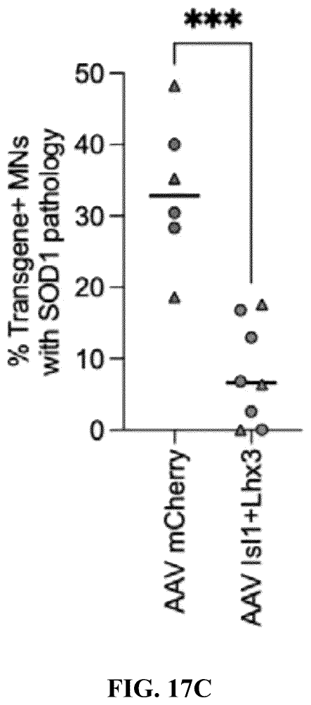

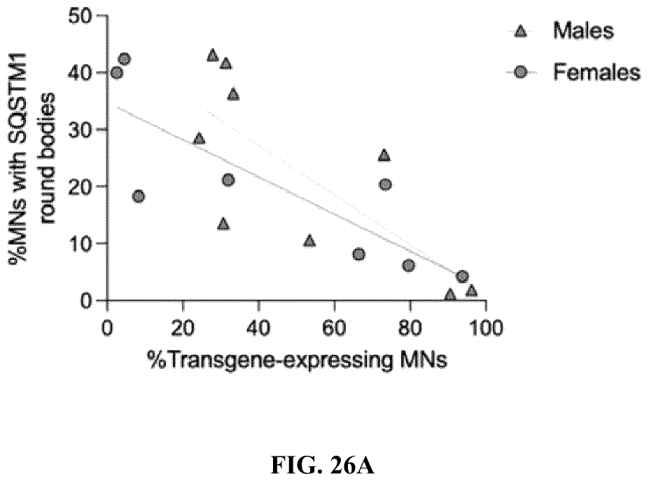

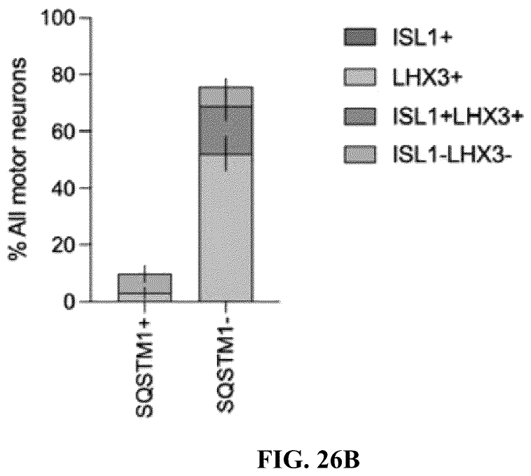

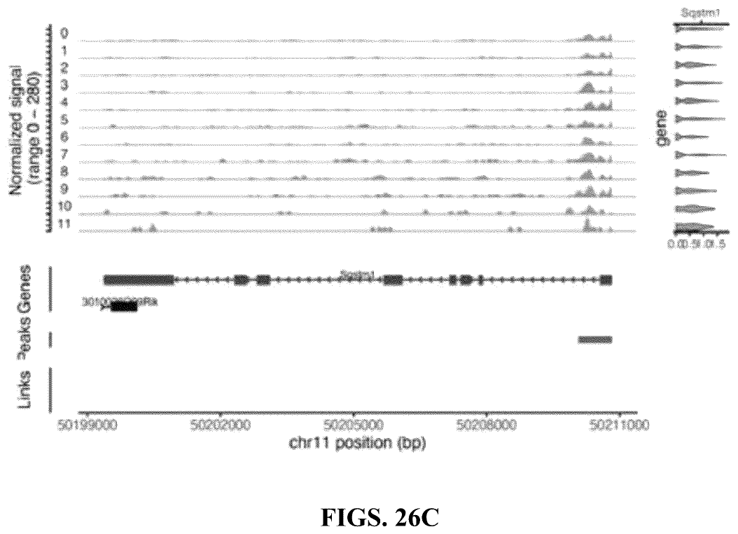

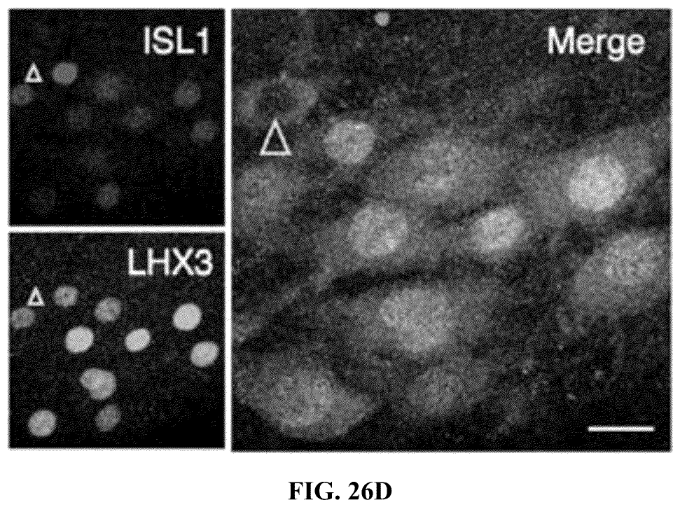

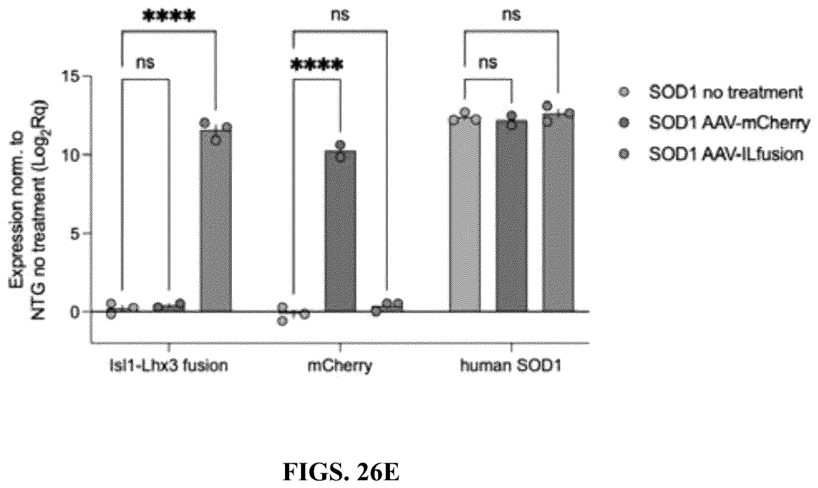

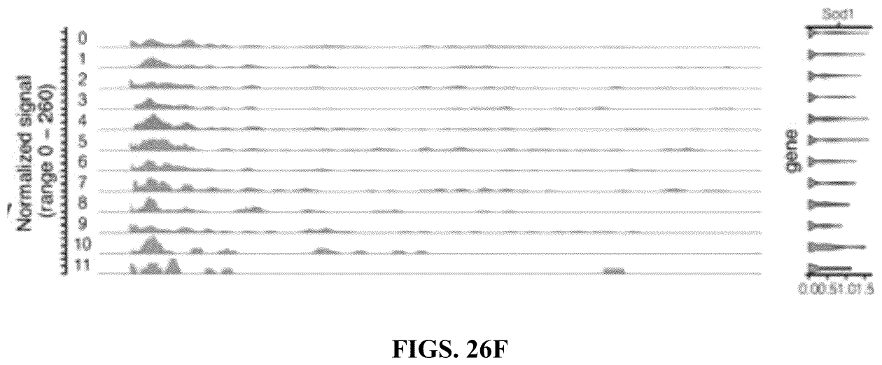

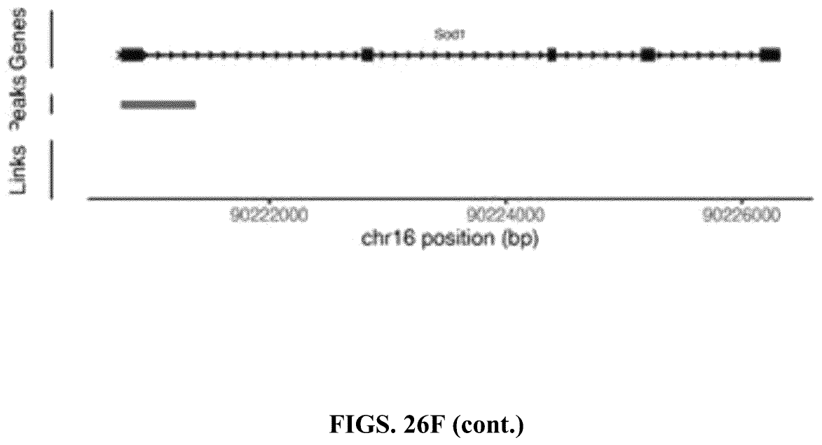

We evaluated the effects of ISL1 and LHX3 re-expression across a range of viral doses (6.37E⁺10-3.69E⁺11 vg/animal) on SQSTM1 round body formation in SOD1^{G93A} animals treated with AAV-Isl1+AAV-Lhx3 at P1 and analyzed at P45. Across all doses, we found that the percentage of transgene-expressing motor neurons exhibiting SQSTM1 round bodies was strongly reduced in AAV-Isl1+Lhx3-treated animals compared to mCherry (2.86% vs. 36.18%; p<0.0001, FIGS. 6A, 17A). We also found that the total number of motor neurons per hemisection that exhibited SQSTM1 pathology was dose-dependent and correlated significantly with the percentage of transgene-expressing motor neurons at this time point (R²=0.5910, p=0.0003; FIG. 26A). When the percentage of transgene-expressing motor neurons exceeded ˜80%, SQSTM1 round bodies were almost completely abrogated (FIGS. 6A, 17A, 26A-B), pointing to a cell-autonomous effect of ISL1 and LHX3 re-expression on SQSTM1 pathology. AAV-Isl1+AAV− Lhx3 treatment did not reduce the average number of motor neurons per hemisection (FIG. 20B), nor did it prevent the expression of Sqstm1 (FIGS. 6A, 26C). Furthermore, the percentage of motor neurons exhibiting SQSTM1 pathology did not differ between AAV-mCherry-treated and uninjected controls (FIG. 3a), indicating that round body formation was not affected by AAV transduction itself, nor was it exacerbated by the ectopic expression of mCherry. Taken together, these findings suggest that the effect of ISL1 and LHX3 re-expression on SQSTM1 round body formation in transduced motor neurons was a direct reflection of enhanced resilience to stress resulting from mutant SOD1 expression.

### ISL1 and LHX3 Factors Suppress the Formation of Pathological SOD1⁺ Structures in Spinal Motor Neurons

SQSTM1 has been shown to selectively bind mutant SOD1 and actively sequester it into insoluble cytoplasmic inclusions of ubiquitinated proteins that intensify over time in spinal motor neurons in SOD1 mutant mouse models^{16,17}. Mutant, misfolded SOD1 also accumulates in large, vacuolated structures that likely represent distended mitochondria²⁰. To investigate the effects of ISL1 and LHX3 re-expression on these processes, we used an antibody specific to the human transgene to quantify the incidence of SOD1-positive cytoplasmic structures. Compared to SQSTM1 round bodies, which were pronounced by P45, we observed that SOD1⁺ structures were more readily detectable in motor neurons at later stages of the disease (FIG. 17B), consistent with previous reports²¹. By P75, when SOD1 pathology was severe in untreated or AAV-mCherry-treated animals, we found that AAV-Isl1+AAV-Lhx3 treatment reduced the incidence of SOD1⁺ structures by >3-fold in transgene-expressing motor neurons (p=0.0042; FIG. 17C).

To evaluate whether the effects of ISL1 and LHX3 reexpression on cytoplasmic SOD1⁺ structures might be due to the repression of transgenic SOD1 expression, we injected SOD1^{G93A} animals at P1 with an AAV encoding an ISL1-LHX3 fusion protein under the control of the ChatE and collected RNA from whole spinal cord lysates at P14, when expression of both factors was high (FIG. 26D). Using a primer pair that is specific to the human SOD1 transgene²², we found by qPCR that there was no difference in transgenic SOD1 expression between animals treated with AAV-Isl1-Lhx3-fusion compared to AAV-mCherry-treated or untreated controls (FIG. 26E). Furthermore, AAV-Isl1+AAV-Lhx3 treatment did not alter the expression of endogenous Sod1 (FIG. 26F). Together, these findings support the idea that ISL1 and LHX3 re-expression in mature motor neurons can prevent the cascade of proteostatic deficits that stems from the overexpression of mutant SOD1 and drives disease progression.

### ISL1 and LHX3 Delay the Onset of Clinical Disease in ALS Mice and Protect CHAT+ Motor Neurons

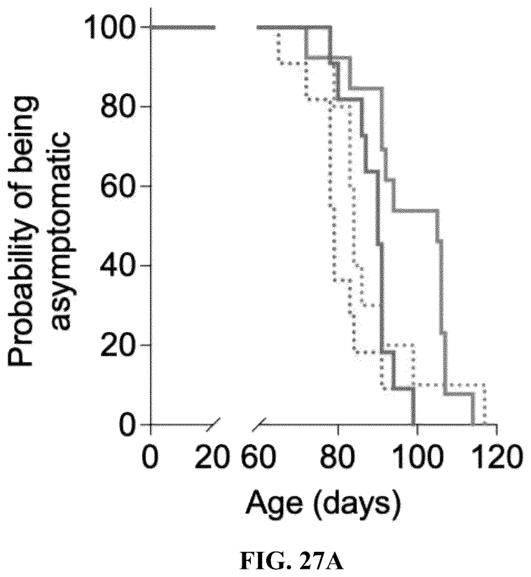

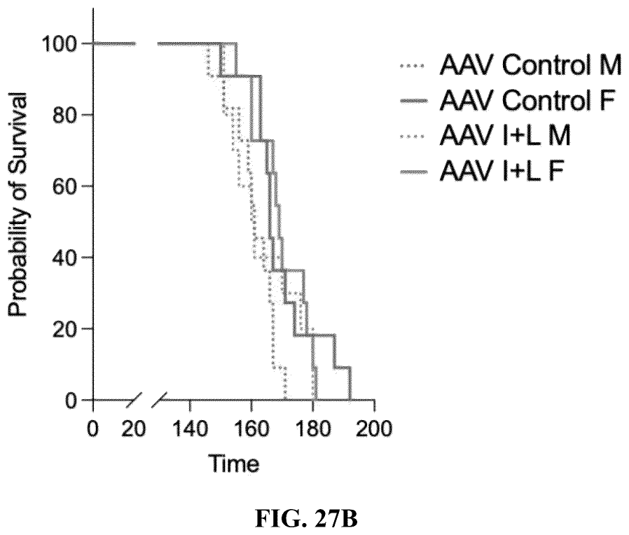

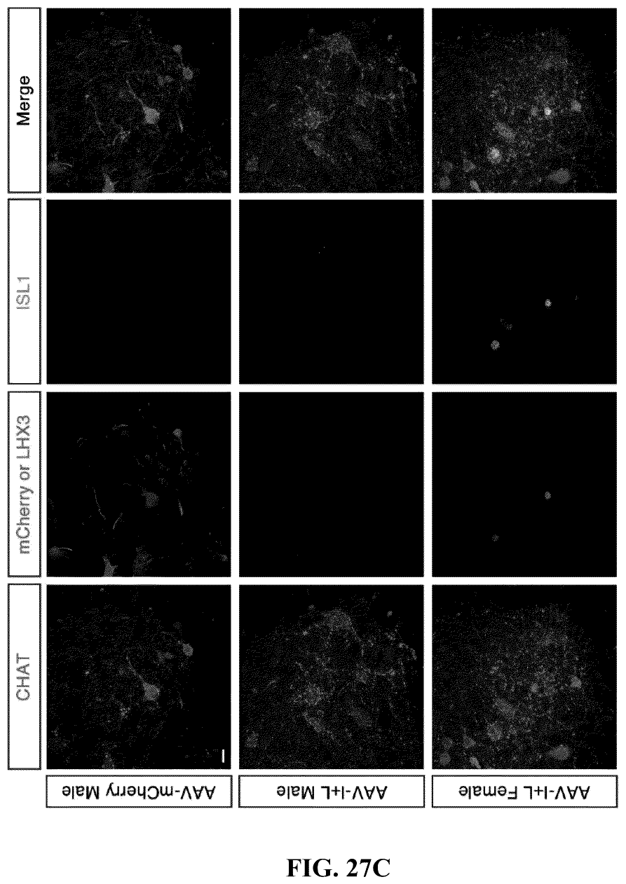

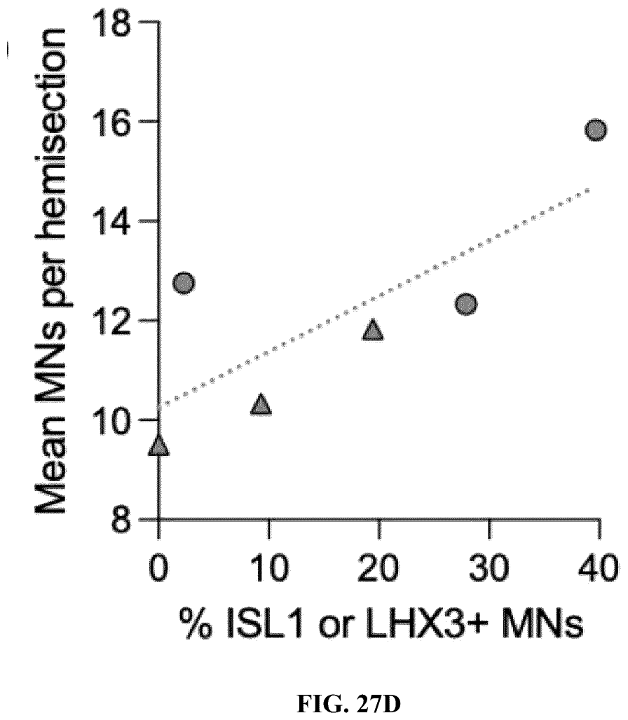

Given that protein dyshomeostasis plays an early and causal role in motor neuron degeneration and clinical disease onset in SOD1 mutant mice^{15,17,23}, we set out to determine whether ISL1 and LHX3 re-expression would impact these key phenotypic readouts. We first evaluated the efficacy of AAV-Isl1+AAV-Lhx3 vs. AAV-mCherry administered at P1 under low titer conditions (7.56E⁺10 vg/animal), where ˜20% of CHAT⁺ motor neurons show transgene expression by P45 (FIG. 5D). AAV-mCherry was used as a control. Animals were monitored over time for the appearance of fine hindlimb tremors, which marks the onset of clinical symptoms in this model²⁴. We found that AAV-Isl1+AAV-Lhx3 treatment significantly delayed the appearance of tremors in females from P90 to P105 (p=0.0036; FIG. 27A). There was a similar trend in males, though it was not significant (median delay of 5 days from P79 to P84; p=0.1138), perhaps because male SOD1^{G93A} mice generally show more rapid progression of clinical phenotypes than females^{25,26}. Animals were monitored through endstage, and there was no effect of AAV-Isl1+AAV-Lhx3 treatment on lifespan (FIG. 27B). We collected spinal cord tissues from a small subset of animals in this study to evaluate transgene expression, and, consistent with the rapid downregulation of ISL1 and LHX3 observed after low titer transduction in control mice, we found that expression in surviving CHAT+ motor neurons at endstage was generally low (FIG. 27C). Nevertheless, there was a trend toward a correlation between the percentage of transgene-expressing motor neurons and the mean number of CHAT-expressing motor neurons overall (R²=0.6200; p=0.0630; FIG. 27D).

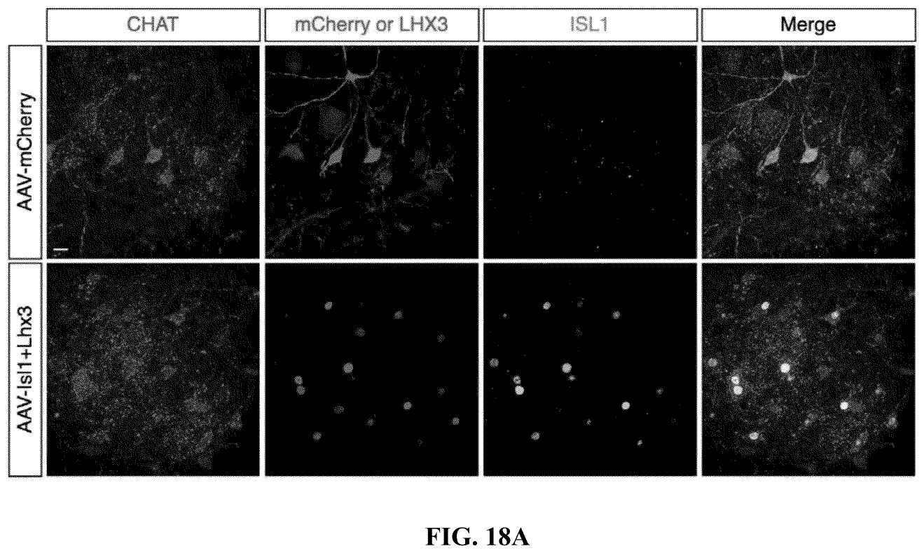

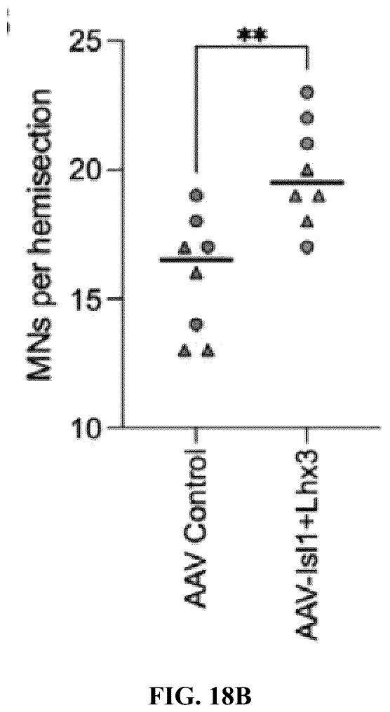

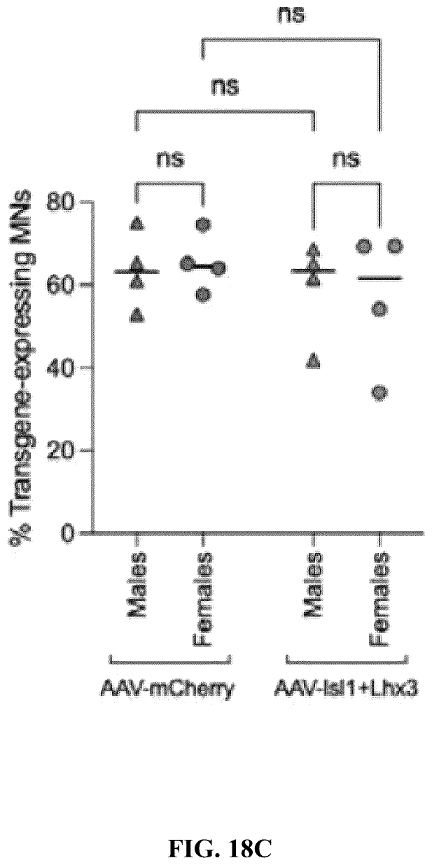

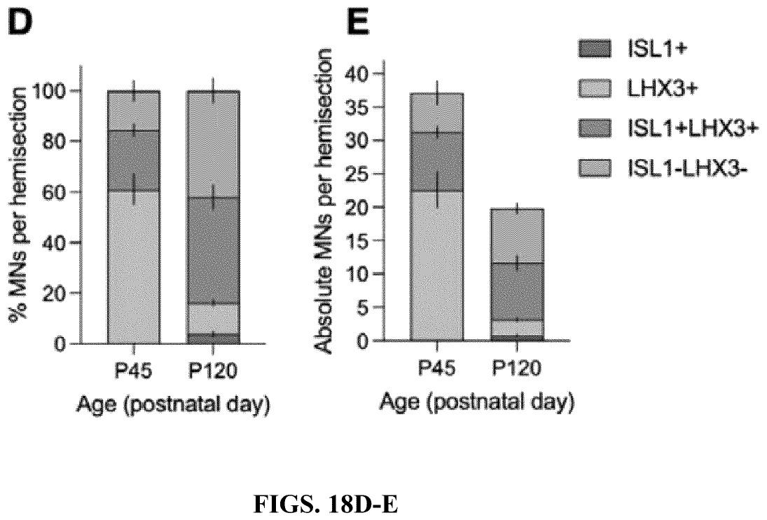

These preliminary findings in animals treated under low titer conditions suggested that increasing transgene expression could further enhance the persistence of CHAT-expressing motor neurons. To test this idea, we treated a second cohort of SOD1 animals at P1 with AAV-mCherry as above (3.19E+10 vg/animal, n=4 males+4 females) or with AAV-Isl1+AAV-Lhx3 at high titer (1.49E+11 vg/animal, n=4 males+4 females) and quantified CHAT+ motor neurons at P120, when most SOD1^{G93A} animals show overt clinical symptoms under control AAV-mCherry conditions (FIG. 27A) and the loss of CHAT+ motor neurons under control conditions is profound²⁰. Given the progressive downregulation of the transgenes, even under high titer treatment conditions, we reasoned that this earlier time point might allow us to capture more ISL1 and LHX3-positive motor neurons than we could at endstage. When we quantified the overall number of CHAT⁺ motor neurons per hemisection, we found that there was a significant increase among AAV-Isl1+AAV-Lhx3 vs. AAV-mCherry-treated animals (p=0.0024, FIGS. 18A-B), suggesting that the effects of ISL1 and LHX3 re-expression on earlier proteostatic defects translate into long-term motor neuron protection.

Next, when we looked at the distribution of transgene-expressing motor neurons across treatments, we found that they were very similar between AAV-mCherry and AAV-Isl1+AAV-Lhx3 (65% vs. 63%, respectively; p=0.2597), and that there were no sex differences in the percentage of transgene-positive cells under either condition (FIG. 18C). Among AAV-Isl1+AAV-Lhx3-treated animals, the proportion of motor neurons that were co-positive for ISL1 and LHX3 was higher at this time point than at P45, prior to the onset of neurodegeneration (41.81% at P120 vs. 23.44% at P45; FIG. 18D), while the absolute number of co-positive motor neurons per section was essentially unchanged (7 vs. 8 cells, respectively; FIG. 18E). Collectively, these findings suggest that ISL1 and LHX3 reexpression might offer long-term motor neuron protection, and that sustained co-expression of both transgenes may be critical to this effect.

### Discussion

This study describes a highly targeted reprogramming approach to rejuvenate susceptible neuronal types and suppress adult-onset neurodegenerative diseases. In contrast to methods that rejuvenate cells through the overexpression of general pluripotency factors (OCT4, SOX2, and KLF4), our approach combines native motor neuron factors with a motor neuron-specific expression system to achieve more targeted and biologically relevant reprogramming of the cells most vulnerable to disease. Heterochronic expression of ISL1 and LHX3 in mature neurons leads to changes in both chromatin accessibility and gene expression in alpha and type 3 motor neurons that are consistent with a partial reversion to a younger cellular state. Functionally, we found that ISL1 and LHX3 re-expression at an early postnatal stage can prevent key histological and clinical phenotypes in a mouse model of ALS, an incurable, adult-onset motor neuron degenerative disease.

While AAV-Isl1 and AAV-Lxh3 appeared to be expressed in all three major motor neuron subtypes, changes in gene expression and chromatin accessibility unexpectedly were limited to—and diverged between—alpha and type 3 motor neurons. The mechanisms underlying the subtype-selective effects of these motor neuron selector transcription factors are not immediately clear, but may be related to basal differences in genome accessibility that facilitate or prevent ISL1 and LHX3 binding. Alternatively, each motor neuron subtype may endogenously express other transcription factors or co-factors that modulate the activity of ectopic ISL1 and LHX3. Nevertheless, the preferential effects of re-expressed ISL1 and LHX3 on alpha over gamma motor neurons on gene expression may be particularly important in the context of ALS, where alpha motor neurons are highly vulnerable to disease while gamma motor neurons are resilient²⁷. The fate of type 3 motor neurons in ALS is unknown, but single nucleus RNAseq and ATACseq studies of motor neurons in vivo, including our own, can inform future strategies to selectively interrogate type 3 motor neurons in the context of disease.

The widespread gene expression changes induced by ISL1 and LHX3 re-expression in vulnerable motor neurons point to several potential mechanisms that may explain the beneficial effects of these factors in SOD1^{G93A} mice. Previous work in corticospinal tract (CST) motor neurons supports the idea that reversion to an immature transcriptional state is protective. Following spinal cord lesion, CST motor neurons reactivate a host of developmentally-regulated genes as part of an endogenous response to injury, and prolonging this immature transcriptional state through treatment with grafts of spinal neural progenitor cells can promote axon regrowth and restore limb function²⁸. We noted that many of the shared, upregulated alpha and type 3 DEGs populating GO terms associated with neurogenesis were also upregulated in regenerating CST motor neurons. These include genes involved in axon guidance and branching (Unc5d, Dcc, and Fgf13), as well as genes involved in synaptic plasticity (Grik1 and Rgs7). Thus, ISL1 and LHX3 may be reactivating a broadly pro-regenerative gene expression program.

Additionally, in vivo electrophysiology studies in SOD1^{G93A} mice have shown that alpha motor neurons are hypoexcitable prior to the onset of neurodegeneration^{29,30} and that enhancing motor neuron excitability using chemogenetic strategies can restore postsynaptic function by increasing endogenous glutamate receptor trafficking and can attenuate both SQSTM1 and mutant SOD1 accumulation³¹. Though the pathway(s) through which enhanced motor neuron activity might resolve these proteinopathies is not yet known, we observed a significant enrichment of synaptic genes among shared alpha and beta DEGs, including a number of glutamate receptor subunits (e.g., Gria4). Additionally, there are several DEGs associated with protein turnover and degradation, including the developmentally-regulated E3 ubiquitin ligases Pam, Wsb1, and March1, as well as modulators of intracellular vesicle biogenesis and trafficking such as Lyst, Clvs2, and Sbf1. Future studies may determine whether AAV-Isl1+AAV-Lhx3 can modulate motor neuron excitability, and how this might influence proteostasis or survival.

The individual contributions of each transcription factor to the attenuation of ALS pathologies also warrants further investigation. Under similar low titer transduction conditions, ChatE-driven mCherry was strongly and continuously expressed in most motor neurons at all ages examined, while ISL1 and LHX3 expression dropped precipitously after ˜2 weeks, suggesting that the re-expressed transcription factors are either silenced or degraded in a selective manner. Increasing viral titers helped sustain LHX3 but not ISL1 expression, and, accordingly, most of the transgene-expressing cells that were devoid of SQSTM1 or SOD1 pathology in SOD1^{G93A} mice were ISL1⁻ LHX3+. At the same time, most of the remaining CHAT-positive, transgene-positive motor neurons at later stages of the disease were ISL1⁺LHX3⁺. Without being bound by theory, these findings raise the question of whether high-titer AAV-Lhx3 on its own may be effective in attenuating early histopathological phenotypes but insufficient in preventing motor neuron loss. The efficacy of each factor alone and in combination following administration at later postnatal timepoints, including after symptom onset can be evaluated to inform whether motor neuron-targeted interventions can be beneficial at late stages of the disease when motor neuron degeneration is underway and non-cell-autonomous factors such as neuroinflammation contribute to clinical progression³².

In summary, our work demonstrates that heterochronic re-expression of embryonic selector transcription factors in postnatal animals can cell type-specifically increase the resilience of susceptible neurons and ameliorate cellular and behavioral pathologies in adult-onset neurodegenerative diseases.

Materials and Methods
ChatE
ChatE was amplified from genomic DNA of wildtype C57BL6/J mice using
primers containing ~28 bps of homology to the pAAV backbone (see below). The first and
last 21 bps were used as primers to amplify this sequence. The sequence of ChatE is SEQ
ID NO: 1:
CAGTGAGCTTCATTATCACCTAACAGCTTCAGAGTGGGTGGTGGGTTTTGGATGAC
AACCTTTCTTCTCATTTTATTCAGTGGCCACACCGTGGCCTTAGTCTGATAAACCA
AAAACCTGCTCCATTATGAATCAGTGCTGTGGGGAGTGGGTAGAGAGTGTGAAGT
TCTGGGGGGGGGAGTCTGGAGAGAGGGTGGGAGCAGCCATTCTGCAGCAGTGCC
TTCTTGGGGTCATGGGTCTGTAGGTGCTGCTGTGGAGGGAGAGATCAGCCTATTCT
GGCTTCATTTCTGAGCTGCAAACTGCCTGGGTGTCTGGAGAAGCAGGTTGGCGTG
GTGGTTAGCAGTGCGTGGGCGGGGTTGCCCGCTCTTGATTTATGATTTCTTTGTCT
CTGTGGAAGCACTTAAGTGCAGGCTTTAGTTCCAATGACACTCAGGAGCCTCTGG
ATTCCAGCACTGGGGATGGGGGTGGGGTAGAACGTTCTCAGGCCTCACCAACCCC
TCCCCTGTGTGCTGCCTTTGGGAGAGTCCCAAGGCTTCAGCATTACTTAATTAATT
AGGCCTCTACTGCTACATAGGCTCAGATTCAAAAGAACAGAGTGGCCCACGTCAG
CCATTCCCGGAAAAGTCTGATGGCTGGAAGCCAGAGGACTATGTGTCTGCCTTGC
TGCCCTTGGCCAGCCCATCCTGAATGCCCAGACTCGGACAATGGAGTAGGTACAG
AAGGGTAAAGACAGTGTCTTCTGTACCAGTAAGTGGGCCCTGATCTGCTCTCTACA
GCTTCCAGAGAAAGGGCCTGGCCAATGAGCGGCCTTTTGAGTAGCAGATACCTCA
CATGCATTCTGATAGAAAGCCTGGCCCCAGATCACTGTGACTTTAGCCCTCAGGTT
TCTTTTGCACTTCAATTCAATGACTTCTTGAGGTTCATTTCCCTCTCCAAGATTTGC
CACAGACCAGTGGTTCTCAACCTGTGGGTCACACCTCCTTTGGGGAAATTGAATG
A (SEQ ID NO: 1).
Cloning pAAVs
The pAAV backbone contained an AAV2 ITR, a cloning site, a mini promoter
(TAGAGGGTATATAATGGAAGCTCGACTTCCAG) (SEQ ID NO: 6), a chimeric intron,
an mCherry sequence, a WPRE sequence, a SV40 poly(A) signal, and a second AAV2 ITR.
To add ChatE, pAAV was linearized by digesting at the cloning site with Kpn1. ChatE was
inserted into linearized pAAV with Gibson cloning.
To generate the pAAV-Isl1 and pAAV-Lhx3 constructs, human Isl1 and Lhx3
cDNAs were amplified using primers to add ~28 bps of homology to the pAAV-ChatE
backbone for Gibson cloning (Lhx3 F primer:
cctttctctccacagggcaaagaattggatccatggaggcgcgcggggag (SEQ ID NO: 16); Lhx3 R primer:
agaggttgattatcgataagcttgatatcttagaactgagcgtggtctac (SEQ ID NO: 17); Isl1 F primer:
cctttctctccacagggcaaagaattggatccatgggagacatgggagat (SEQ ID NO: 18); Isl1 R primer:
agaggttgattatcgataagcttgatatcctatgcctcaataggactggc (SEQ ID NO: 19)). The pAAV backbone
containing the ChatE and mCherry was then digested with BamHI and EcoRV to remove the
mCherry and replace it with either Isll or Lhx3.
To generate the pAAV-Isl1-Lhx3-fusion construct, the Isl1-Lhx3 fusion CDS
was PCR amplified from pCMV-Isl1-Lhx3-fusion (gifted by Dr. Soo-Kyung Lee) using
primers
GAATTGGATCCGCCACTCGGAGCAAGCTTGATTTAGGTGACACTATAGAATACA
AGC (SEQ ID NO: 20) and
CATGTCTGCTCGAAGCGTCAGAACTGAGCATGGTCTACTTCA (SEQ ID NO: 21).

The pAAV backbone was linearized by digestion with BamHI and NotI, and the Isl1-Lhx3 fusion CDS was cloned into the digested vector via NEBuilder Hifi Assembly (New England Biolabs), replacing the mCherry CDS and WPRE sequences. The CAGGS-NLS-GFP construct was purchased from Addgene (104061).

### AAV Production

AAVs were generated and titered by following the protocol in Challis et al ³³. No meaningful changes were made to the protocol. We used the PHP.eB cap plasmid and pHelper plasmid in combination with one of the three pAAV plasmids (mCherry, Isl1, or Lhx3) to generate each virus independently. If the titer of the viral preparations fell below 2E⁺12 vg/mL, they were respun through an Amicon filtration device to reduce overall volume and increase the final concentration to the desired titer.

### Animals

All mouse experimental procedures were approved by the Columbia University Medical Center Institutional Animal Care and Use Committee. INTACT mice: SnSeq studies were performed in INTACT mice that were generated and maintained through crosses of Chat-Cre mice (B6.129S-Chat^{tm1(cre)Lowl}/MwarJ, Jax 031661) with Sun1-GFP nuclear reporter mice (B6;129-Gt(ROSA)26Sor^{tm5(CAG-Sun1/sfGFP)Nat}/J; Jax 021039). SOD1^{G93A} mice: All SOD1^{G93A} studies were performed in mice that were heterozygous for the mutant human SOD1^{G93A} transgene on a C57BL/6J background (B6.Cg-Tg(SOD1*G93A)1Gur/J; Jax 004435) and their non-transgenic littermates. Breeding pairs consisted of C57BL6/J females (Jax 000664) and SOD1^{G93A} males purchased from Jackson laboratories, where they were assessed for transgene copy number maintenance. Pups were genotyped at P0 and balanced into treatment groups by sex, genotype, and littermate status.

### Intracerebroventricular AAV Injections:

Intracerebroventricular AAV injections were performed on cryoanesthetized pups at P1 using a 10 μL syringe (Hamilton 7653-01) outfitted with a 0.375-inch 32-gauge needle (Hamilton 7803-04). The needle was inserted at ⅖ths of the distance between the lambda suture and the middle of the eye at a depth of 3 mm, and up to 6 μL of virus was injected per animal.

### Immunohistochemistry

For all immunohistochemical analyses, tissues were collected by transcardial perfusion with 10 mL ice-cold phosphate-buffered saline, followed by 40 mL of 4% paraformaldehyde in 0.1M phosphate buffer. The skull and spine were gross dissected and post-fixed for 18-24 h in 4% paraformaldehyde. The L4-L5 region of the spinal cord was isolated by cutting the spinal cord at the T10 and S1 vertebrae, followed by laminectomy and transection of the spinal cord at the L3 and L6 roots. Spinal cord segments were stored at 4° in PBS containing 0.1% sodium azide until they were sectioned at 70 μm using a vibratome fitted with a disposable blade (Leica). Immunostaining was performed in floating sections using antibodies against CHAT (1:250, Millipore AB144P), ISL1 (1:10000, Thomas M. Jessell Laboratory, Columbia University or 1:5000, R&D Systems AF1837), LHX3 (1:5000, Thomas M. Jessell Laboratory, Columbia University), RFP (1:1000, Thermo Fisher 600-401-379), MNX1 (1:15000, Thomas M. Jessell Laboratory, Columbia University), CADPS2 (1:1000, Thermo Fisher PA5-20518), SQSTM1 (1:500, Abcam ab56416), human SOD1 (1:250, R&D Systems MAG 3418), or NEUN (1:1000, EMD Millipore MAB377). Sections were incubated for 18-24 h in primary antibody solution containing 1% bovine serum albumin, 0.4% Triton, and 0.1% sodium azide in TRIS-buffered saline. Sections were then incubated overnight in a secondary antibody solution containing 0.4% Triton in TRIS-buffered saline using antibodies raised in donkey and conjugated to Alexa Fluor 405, 488, 594, or 647 (Thermo Fisher). Sections were washed, mounted on slides, and coverslipped with Fluoromount-G (Southern Biotech).

### Confocal Microscopy

Immunostained spinal cord sections were visualized at 5× using an epifluorescent microscope (Zeiss Axioscope) and at 40× using a confocal microscope with an oil objective (Zeiss LSM 900). For confocal microscopy, z-sections were collected every 3 μm to achieve ˜15 z sections per hemisection.

### Image Quantification

An experimenter blind to the treatment and genotype of the animals performed quantification of motor neuron survival, MNX1 frequency, SQSTM1 round body formation, SOD1 pathology, and CADPS2 using confocal images. All quantification was performed in L4-L5 sections; sections displaying morphological features of L3 or L6 regions were excluded. Statistical analyses were performed in Prism (v. 10).

Quantification of MNX1⁺ nuclei: The number of MNX1⁺ nuclei among CHAT⁺ cells was manually quantified in 2 hemisections per animal for control untreated animals, or 6 hemisections per animal for AAV-Isl1+AAV-Lhx3-treated animals. ˜200 motor neurons were quantified for each treated animal.

Quantification of motor neuron survival: CHAT⁺ motor neurons were manually quantified in 6-12 hemisections per animal, depending on age and genotype, such that ˜200 motor neurons were quantified per animal.

Quantification of SQSTM1⁺ round bodies: SQSTM1⁺ round bodies ranged from 1.75-4 μm in diameter at P45, consistent with observations by Salvany et al ²⁰. CHAT⁺ motor neurons were considered to be positive for SQSTM1 round bodies if they contained one or more SQSTM1⁺ structures greater than 1.75 μm in diameter. SOD1⁺ structures were more variable in size, ranging from 2-12 μm in diameter at P75. All quantification was performed manually in 6 hemisections per animal, such that ˜200 motor neurons were quantified per animal.

Quantification of SOD1 pathology: SOD1⁺ structures ranged from 2-12 μm in diameter at P75. CHAT+ motor neurons were considered to be positive for SOD1 pathology if they contained one or more SOD1⁺ structures greater than 2 μm in diameter. All quantification was performed manually in 6 hemisections per animal, such that ˜200 motor neurons were quantified per animal.

Quantification of CADPS2: Quantification of CADPS2 positive nuclei and CADPS2 intensity was performed using custom automated macros in ImageJ (source code available upon request). All quantification was performed manually in 6 hemisections per animal, such that ˜200 motor neurons were quantified per animal.

### Bulk Seq Procedure and Analysis

INTACT mice (n=4) were injected under low titer conditions with AAV-Isl1+AAV-Lhx3 (6.38E⁺10 vg/animal) or AAV-mCherry (3.19E+10 vg/animal) at P1. Sun1-GFP⁺ nuclei were collected at P21 using a bead-based IP method described in detail in Patel et al. ². Bulk RNA was isolated from FACs nuclei using TRIzol-based phase separation and the Zymo MicroPrep RNA kit. Libraries were prepared at the MIT MicoBio Center using SMARTer Stranded Total RNA-Seq Kit v2—Pico Input Mammalian kit and sequenced paired end using the 150 nt Nextseq kit. Reads were trimmed for adaptors and low-quality positions using Trimgalore (Cutadapt v0.6.2) ³⁴. Reads were aligned to the mouse genome (mm10) and gene-level counts were quantified using RSEM (v1.3.0) ³⁵ rsem-calculate-expression using default parameters and STAR (v2.5.2b) for alignment. Differential gene expression analysis was performed between injected animals and control P21 gene expression data ² on RSEM gene-level read counts using EdgeR ^{36,37}. For each set of comparisons being performed, genes were filtered for those that are expressed with CPM of ≥5 in all replicates in at least one condition.

### Single Nuc Seq Procedure and Analysis

Sun1-sfGFP-Myc mice were either injected with AAV-Isl1+AAV-Lhx3 under high titer conditions at P1 (3E⁺11 vg/animal, 6 mice, Treatment group) or left untreated (8 mice, Control group). At P21 GFP⁺ nuclei were separately FAC sorted from treatment and control mice. Brachial and lumbar regions of the spinal cord were dissected and nuclei were isolated and sorted following the exact protocol detailed in Patel et al. ². All nuclei collected (˜60,000 from six treatment mice and ˜80,000 from eight control mice) were taken to the Genome Center at Columbia University, where they were counted, and processed using 10× multiome kit and 10× Chromium. Data was pre-processed using CellRanger by the Genomics Core. Alignment of sequencing reads were performed using both the standard mm10 genome and a custom genome that included 4 extra genes present in the AAV driven mRNA—the hIsl1 and hLhx3 coding sequences, chimeric-intron, and WPRE. Standard Seurat and Signac pipelines were then used to cluster and analyze the data. High-quality cells were first filtered using the following parameters: nCount_ATAC<7e4 & nCount_ATAC>5e3 & nCount_RNA<25000 & nCount_RNA>5000. Seurat objects were created from the remaining 7395 Control and 5108 Treatment cells. The two objects were integrated using canonical correlation analysis based on shared anchors identified in the snRNAseq data. 27 clusters were identified (dims=1:20, resolution=0.5), each of which included both control and treatment nuclei. A previous study that performed snRNAseq on GFP⁺ nuclei from adult Chat-Cre; Sun1− sfGFP-Myc mice found clusters that consisted of Gad1 expressing GABAergic neurons, Slc17a6 expressing glutaminergic neurons, in addition to Chat expressing cholinergic neurons ⁸. Consistent with this previous study, we find a similar distribution of neurons in our clusters. Among the cholinergic clusters, we could identify three distinct motor neuron clusters, which correspond to alpha motor neurons, gamma motor neurons, and beta-like type 3 motor neurons based on markers identified in previous single nuclei RNAseq data ^{8,9}. Cholinergic skeletal motor neurons (2094 cells) and all other cholinergic cells (1908) were then subsetted separately into new objects and reclustered without the use of canonical correlation-based integration (dims=1:15 for both, resolution=0.5). WPRE transcripts are used to show which clusters express AAV because Isl1 and Lhx3 transcripts could not be accurately quantified. Human Isl1 and Lhx3 genes were driven by ChatE-AAV. Sequencing reads that aligned only to the human cDNA were counted as AAV-driven transcripts and reads that align only to the mouse Isl1 and Lhx3 loci counted as endogenous; however any reads that aligned to both human and mouse genes were thrown out, making the quantification of these genes inaccurate.

All differential gene expression was performed using FindMarkers with a minimum log 2FC of 0.25 and an adjusted p-value of 0.05. To identify genes that might be generally responsive to AAV treatment rather than to the specific effects of ISL1 and LHX3, we compared gene expression levels between AAV-mCherry-treated INTACT mice from the bulk seq dataset above (n=4 animals, pooled for 1 biological replicate) vs. untreated animals at P21 from the bulk seq dataset generated in ² (n=6-8 animals pooled per replicate for 3 biological replicates). Any genes where the level of expression in AAV-mCherry-treated animals fell above 2 standard deviations of the level of expression in untreated animals were considered to be sensitive to AAV-mCherry and were excluded from DEG and GO analyses of our single nuclear sequencing dataset. 12 Alpha DEGs (6%) and 18 Type 3 DEGs (5%) were excluded as AAV-mCherry-sensitive. Four AAV-mCherry-sensitive DEGs were shared between Alpha and Type 3 MNs (AY036118, Gm42418, Coro6, and Gpr149).

To analyze the ATAC data, peaks were called using Macs2 on clusters in the cholinergic skeletal motor neuron object. Differential peaks were identified using FindMarkers (test.use=‘LR’, latent.vars=‘nCount_peaks’). ClosestFeature was used to identify genes closest to peaks. RegionMatrix and RegionsHeatmap were used to make heatmaps of ATAC-seq reads in FIG. 17C. FindMotifs was used to perform motif enrichment on Jaspar mouse motif dataset (species=10090). GO analysis was performed for up-regulated alpha and type 3 DEGs using the STRING database v12.0 (string-db.org) against the background of all genes that were detected in the snSEQ dataset (9482 genes). FDRs<0.05 were considered significant.

qPCR for SOD1 in Isl1+Lhx3 Fusion

Total RNA was extracted from whole spinal cords of an uninjected non-transgenic mouse, uninjected SOD1 mice (n=3), or SOD1 mice injected with AAV-mCherry (n=2) or AAV-Isl1+Lhx3-fusion (n=3) using TRIzol Reagent (Thermo Fisher Scientific) according to the manufacturer's instructions. RNA concentration and purity were assessed using a NanoDrop spectrophotometer (Thermo Fisher Scientific). Genomic DNA was removed using the Heat&Run gDNA Removal Kit (Arctic Enzymes) according to the manufacturer's instructions. Complementary DNA (cDNA) was synthesized from 5 μg of RNA using the SuperScript IV Kit (Thermo Fisher Scientific) and Oligo-dT following the manufacturer's protocol.

Quantitative PCR was performed using the Applied Biosystems QuantStudio 3. The reaction mix consisted of PowerUp SYBR Green PCR Master Mix (Thermo Fisher Scientific), 0.5 pmol forward and reverse primers, and ˜60 ng cDNA in a total volume of 20 μL, and prepared in technical triplicate. AAV-mCherry was detected by WPRE primers (F: GGCTGTTGGGCACTGACAAT (SEQ ID NO: 22), R: CCGAAGGGACGTAGCAGAAGG (SEQ ID NO: 23)), AAV-Isl1+Lhx3-fusion was detected using primers spanning the junction between each gene and do not detect endogenous Isl1 or Lhx3 (F: TCCACTGGCAGTGAAGTAGCATC (SEQ ID NO: 24), R: AAGGATGAAACGGTCCAAGATGTG (SEQ ID NO: 25)). hSOD1 transgene expression was detected by primers that only amplify the transgene (F: CATCAGCCCTAATCCATCTGA (SEQ ID NO: 26), R: CGCGACTAACAATCAAAGTGA (SEQ ID NO: 27)). The thermal cycling conditions were as follows: an initial denaturation at 95° C. for 10 min, followed by 40 cycles of denaturation at 95° C. for 15 sec, annealing and extension at 60° C. for 1 min. Relative expression levels of target genes were normalized to GAPDH (F: CAACAGGGTGGTGGACCTC (SEQ ID NO: 28), R: GGGTGGTCCAGGGTTTCTTA (SEQ ID NO: 29)) and expression in the uninjected non-transgenic mouse using the ΔΔCt method. Data were analyzed using GraphPad Prism. For conditions with three biological replicates, statistical significance was determined using one-way ANOVA, with a p-value of <0.05 considered significant.

### Tremor Analysis in SOD1^{G93A} Mice:

For analysis of clinical onset, SOD1^{G93A} animals (n=10-13 per treatment per sex) were monitored for the appearance of hindlimb tremors on a daily basis beginning at P40. Tremors were assessed qualitatively as per Hatzipetros et al. ²⁴ by an experimenter blind to the genotype and treatment of the animals. The probability of tremor onset in AAV-mCherry vs AAV-Isl1+AAV-Lhx3-treated SOD1^{G93A} mice was compared by Log-rank (Mantel-Cox) tests in Prism (v.10). Separate analyses were performed for male vs. female animals.

### REFERENCES FOR EXAMPLE 9

1. Masrori, P. & Damme, P. V. Eur. J. Neurol. 27, 1918-1929 (2020).
2. Patel, T. et al. Nat Commun 13, 5427 (2022).
3. Closser, M. et al. Neuron (2021).doi:10.1016/j.neuron.2021.10.014
4. Mazzoni, E. O. et al. Nat Neurosci 16, nn.3467 (2013).
5. Rhee, H. S. et al. Neuron 92, (2016).
6. Lee, S. et al. Proc National Acad Sci 109, 3383-3388 (2012).
7. Liu, M.-L., Zang, T. & Zhang, C.-L. Cell Reports 14, 115-128 (2016).
8. Alkaslasi, M. R. et al. Nat. Commun. 12, 2471 (2021).
9. Blum, J. A. et al. Nat Neurosci 24, 572-583 (2021).
10. Martins, L. F. et al. Neuron 110, 4090-4107.e11 (2022).
11. Marquardt, T. et al. Cell 121, 127-139 (2005).
12. Gallarda, B. W. et al. Science 320, 233-236 (2008).
13. Jaworski, A., Long, H. & Tessier-Lavigne, M. J Neurosci 30, 9445-9453 (2010).
14. Kumar, A. V., Mills, J. & Lapierre, L. R. Front. Cell Dev. Biol. 10, 793328 (2022).
15. Rudnick, N. D. et al. Proc National Acad Sci 114, E8294-E8303 (2017).
16. Gal, J., Ström, A.-L., Kilty, R., Zhang, F. & Zhu, H. J Biol Chem 282, 11068-11077 (2007).
17. Mitsui, S. et al. Mol Brain 11, 30 (2018).
18. Trist, B. G. et al. Acta Neuropathologica Commun 10, 122 (2022).
19. Fecto, F. et al. Arch. Neurol. 68, 1440-1446 (2011).
20. Salvany, S. et al. Brain Pathol. 32, e13078 (2022).
21. Watanabe, M. et al. Neurobiol. Dis. 8, 933-941 (2001).
22. Alexander, G. M. et al. Mol Brain Res 130, 7-15 (2004).
23. Saxena, S., Cabuy, E. & Caroni, P. Nat Neurosci 12, nn.2297 (2009).
24. Hatzipetros, T. et al. J Vis Exp (2015). doi:10.3791/53257
25. Miana-Mena, F. J. et al. Amyotroph Lateral Sc 6, 55-62 (2009).
26. Scott, S. et al. Amyotroph Lateral Sc 9, 4-15 (2009).
27. Lalancette-Hebert, M., Sharma, A., Lyashchenko, A. K. & Shneider, N. A. Proc National Acad Sci 113, E8316-E8325 (2016).
28. Poplawski, G. H. D. et al. Nature 581, 77-82 (2020).
29. Delestrée, N. et al. J Physiology 592, 1687-1703 (2014).
30. Martinez-Silva, M. de L. et al. Elife 7, e30955 (2018).
31. Ba̧czyk, M. et al. J. Exp. Med. 217, e20191734 (2020).
32. Liu, J. & Wang, F. Front Immunol 8, 1005 (2017).
33. Challis, R. C. et al. Nat Protoc 14, 379-414 (2019).
34. Martin, M. EMBnetJ. 17, 10-12 (2011).
35. Li, B. & Dewey, C. N. BMC Bioinform. 12, 323 (2011).
36. Robinson, M. D., McCarthy, D. J. & Smyth, G. K. Bioinformatics 26, 139-140 (2010).
37. McCarthy, D. J., Chen, Y. & Smyth, G. K. Nucleic Acids Res. 40, 4288-4297 (2012).

What is claimed is:

1. A method for treating Amyotrophic Lateral Sclerosis (ALS) in a subject in need thereof, the method comprising:
administering to the subject a composition comprising adeno-associated viruses (AAVs), wherein the AAVs comprise a nucleic acid sequence comprising an enhancer sequence and encoding one or more transcription factors that control gene expression in nascent motor neurons, wherein the enhancer drives a motor-neuron specific expression of the one or more transcription factors.

2. The method of claim 1, wherein the enhancer sequence comprises SEQ ID NO: 1.

3. The method of claim 1, wherein the one or more transcription factors is Lhx3, Isl1, Phox2a, Sox2, or Oct4.

4. The method of claim 1, wherein the AAVs comprise a nucleic acid sequence encoding at least two transcription factors, wherein the at least two transcription factors are Isl1 and Lhx3; Phox2a and Isl1; or Oct4 and Sox2.

5. The method of claim 1, wherein the AAVs comprise a nucleic acid sequence encoding two transcription factors, wherein the two transcription factors are Isl1 and Lhx3; Phox2a and Isl1; or Oct4 and Sox2.

6. The method of claim 5, wherein the two transcription factors are Isl1 and Lhx3.

7. The method of claim 6, wherein the expression of the two transcription factors reactivates MNX1.

8. The method of claim 1, wherein the motor neurons are spinal motor neurons.

9. The method of claim 1, wherein the AAVs are capable of penetrating the blood-brain barrier.

10. The method of claim 1, wherein administration of the AAVs reduces disease-related proteinopathies in the motor neurons, reduces the formation of p62+ aggregates in the motor neurons, reduces the incidence of SQSTM1-positive round bodies, reduces the formation of SOD1+ aggregates in the motor neurons, reduces neuroinflammation in the vicinity of motor neurons, reduces Iba1+ microglia activation in the vicinity of the motor neurons, or a combination thereof.

11. The method of claim 1, wherein administration of the AAVs ameliorates clinical phenotypes of ALS or delays symptom onset of ALS.

12. A method for treating Amyotrophic Lateral Sclerosis (ALS) in a subject in need thereof, the method comprising:
administering to the subject a first composition comprising adeno-associated viruses (AAVs) and a second composition comprising AAVs, wherein AAVs of the first composition comprise a nucleic acid sequence comprising an enhancer sequence and encoding a first transcription factor that controls gene expression in nascent motor neurons and wherein the enhancer drives a motor-neuron specific expression of the first transcription factor,
wherein AAVs of the second composition comprise a nucleic acid sequence comprising an enhancer sequence and encoding a second transcription factor that controls gene expression in nascent motor neurons wherein the enhancer drives a motor-neuron specific expression of the second transcription factor.

13. The method of claim 12, wherein the enhancer sequence comprises SEQ ID NO: 1.

14. The method of claim 12, wherein the first transcription factor is Isl1 and the second transcription factor is Lhx3, the first transcription factor is Phox2a and the second transcription factor is Isl1, or the first transcription factor is Oct4 and the second transcription factor is Sox2.

15. The method of claim 12, wherein the first transcription factor is Isl1 and the second transcription factor is Lhx3.

16. The method of claim 15, wherein the re-expression of the first and second transcription factors reactivates MNX1.

17. The method of claim 12, wherein the motor neurons are spinal motor neurons.

18. The method of claim 12, wherein the AAVs are capable of penetrating the blood-brain barrier.

19. The method of claim 12, wherein administration of the AAVs reduces disease-related proteinopathies in the motor neurons, reduces the formation of p62+ aggregates in the motor neurons, reduces the incidence of SQSTM1-positive round bodies, reduces the formation of SOD1+ aggregates in the motor neurons, reduces neuroinflammation in the vicinity of motor neurons, reduces Iba1+ microglia activation in the vicinity of the motor neurons, or a combination thereof.

20. The method of claim 12, wherein administration of the AAVs ameliorates clinical phenotypes of ALS or delays symptom onset of ALS.

21. A composition for treating ALS in a subject in need thereof, the composition comprising AAVs,
wherein the AAVs comprise a nucleic acid sequence comprising an enhancer sequence and encoding one or more transcription factors that control gene expression in nascent motor neurons,
wherein the enhancer is capable of driving a motor-neuron specific expression of the one or more transcription factors.

22. The composition of claim 21, wherein the one or more transcription factors are Isl1 and Lhx3, Phox2a and Isl1, or Oct4 and Sox2.

23. A composition for treating ALS in a subject in need thereof,
the composition comprising: a first set of AAVs and a second set of AAVs,
wherein the first set of AAVs comprise a nucleic acid sequence comprising an enhancer sequence and encoding a first transcription factor that controls gene expression in nascent motor neurons and wherein the enhancer is capable of driving a motor-neuron specific expression of the first transcription factor,
wherein the second set of AAVs comprise a nucleic acid sequence comprising an enhancer sequence and encoding a second transcription factor that controls gene expression in nascent motor neurons wherein the enhancer is capable of driving a motor-neuron specific expression of the second transcription factor.

24. The composition of claim 23, wherein the first transcription factor is Isl1 and the second transcription factor is Lhx3, the first transcription factor is Phox2a and the second transcription factor is Isl1, or the first transcription factor is Oct4 and the second transcription factor is Sox2.
